(19) United States
(12) Patent Application Publication
(10) Pub. No.: US 2022/0312793 A1
(43) Pub. Date: Oct. 6, 2022

(54) METHODS OF PROTEIN EXTRACTION AND DOWNSTREAM PROCESSING OF EUGLENA

(71) Applicant: NOBLEGEN INC., Peterborough (CA)

(72) Inventors: Adam J. NOBLE, Peterborough, ON (CA); Somayeh SABOURI, Peterborough, ON (CA); Charles Jonathan CLARKE, Peterborough, ON (CA); Angela SWAIN, Peterborough, ON (CA); Michael Robert Randle CAVERLY, Peterborough, ON (CA); Peeyush MAHESHWARI, Peterborough, ON (CA); James KIRKWOOD, Peterborough, ON (CA); Chonggang ZHANG, Peterborough, ON (CA); Lauren Elizabeth CAMERON, Peterborough, ON (CA)

(21) Appl. No.: 17/618,938

(22) PCT Filed: Jun. 29, 2020
(86) PCT No.: PCT/IB2020/056137

§ 371 Date: Dec. 14, 2021

Related U.S. Application Data
(60) Provisional application No. 62/868,569, filed on Jun. 28, 2019.

(51) Int. Cl. A23J 1/00; A23C 11/06; C12N 1/12; A23L 17/60; A23J 3/14; A23P 10/40

(52) U.S. Cl. CPC A23J 1/006; A23C 11/065; C12N 1/12; A23L 17/60; A23J 3/14; A23P 10/40

(57) ABSTRACT

Embodiments herein are directed to methods for preparing and compositions containing microalgae biomass. Described herein are, for example, methods of preparing a microalgal flour that involve culturing microalgae, concentrating the microalgae into a microalgal biomass sludge, washing the microalgal biomass sludge, adjusting the pH of the microalgal biomass sludge, and drying the microalgal biomass sludge to produce the microalgal flour. Also disclosed are food products supplemented with microalgae biomass, including for example microalgal flour, a protein concentrate, or a protein isolate.

### CROSS-REFERENCE TO RELATED APPLICATIONS

This application claims the benefit of U.S. Provisional Application No. 62/868,569 filed Jun. 28, 2019, which is hereby incorporated by reference in its entirety.

### SUMMARY

Embodiments described herein are directed to methods of preparing a microalgal flour comprising: culturing microalgae, concentrating the microalgae into a microalgal biomass sludge, washing the microalgal biomass sludge, adjusting the pH of the microalgal biomass sludge, and drying the microalgal biomass sludge to produce the microalgal flour.

In embodiments described herein, the method of making protein concentrate comprises: culturing microalgae, bringing the culture to about 3% to about 30% solids, optionally about 10% to about 30% solids, optionally about 5% to about 15% solids, adjusting the culture to a pH of about 6 to about 11, homogenizing the culture, centrifuging the homogenate, and separating the homogenate into three layers, a pellet, a middle layer, and a top layer, wherein the middle layer is the soluble protein. The middle layer is precipitated by adjusting the pH of about 3.5 to about 5.5, optionally about 4 to about 5, optionally incubated at around 22° C. for at least 1 hour, and centrifuged, yielding a heavy phase made of protein concentrate sludge (protein sludge), and a light phase containing acid soluble cellular material and unprecipitated protein, referred to as whey. Optionally, the protein concentrate sludge is diluted with water, optionally an equal weight of water, to create a protein slurry. The pH of the protein slurry is adjusted to about 5.5 to about 8.5, optionally about 6 to about 8. The protein slurry may be spray dried.

In embodiments described herein, the method of making protein isolate comprises: culturing microalgae, bringing the culture to about 3% to about 30% solids, optionally about 10% to about 30% solids, optionally about 5% to about 15% solids, adjusting the culture to a pH of about 6 to about 11, homogenizing the culture, centrifuging the homogenate, and separating the homogenate into three layers, a pellet, a middle layer, and a top layer. The middle layer is precipitated by addition of acid to a pH of about 3.5 to about 5.5, optionally about 4 to about 5, optionally incubated at around 22° C. for at least 1 hour, and centrifuged, yielding a heavy phase made of protein concentrate sludge (protein slurry), and a light phase containing acid soluble cellular material and unprecipitated protein, referred to as whey. The precipitated protein concentrate slurry may be washed and filtered to further increase the protein content in the protein isolate. Optionally, the protein concentrate sludge is diluted with water, optionally an equal weight of water, to create a protein slurry. Optionally, the protein slurry is (a) centrifuged and (b) resuspended in water, where (a) and (b) are optionally repeated one or more times. The pH of the protein sludge or slurry is adjusted to about 5.5 to about 8.5, optionally about 6 to about 8. The protein slurry may be spray dried.

Embodiments described herein are directed to compositions comprising about 5% to about 100% microalgae biomass.

Embodiments described herein are directed to food products comprising about 0.1% to about 100% microalgae biomass and an edible ingredient.

### DESCRIPTION OF THE DRAWINGS

FIG. 1 depicts the solvent extracted Euglena protein concentrate from left to right: Full fat Protein concentrate, Protein concentrate defatted with hexane, Protein concentrate defatted with isopropanol, Protein concentrate defatted with ethanol.

FIG. 2 depicts the solvent extracted Euglena protein flour from left to right: Full fat Protein Flour, Protein Flour defatted with hexane, Protein Flour defatted with isopropanol, Protein Flour defatted with ethanol.

FIG. 3 displays an image of the Coconut Citrus Protein Bar.

FIG. 4 displays an image of the Dark Chocolate, Almond and Cranberry Protein bar.

FIG. 5 displays rolled pasta dough containing Euglena flour from top to bottom: Control with 0% Euglena Flour, 10% Euglena Flour, 20% Euglena Flour and 30% Euglena Flour.

FIG. 6 displays rolled Gluten Free Pasta Dough with Euglena flour from top to bottom: Control with 0% Euglena flour, 10% Euglena flour and 20% Euglena flour.

FIG. 7 displays rolled eggless pasta dough with Euglena flour top: Control with 0% Euglena flour and bottom: 20% Euglena flour.

FIG. 8 displays Versions 1-3 of the extruded product with protein rich Euglena flour and pea protein. A displays version 1 formulation, B is version 2 and C is version 3 of the extruded product.

FIG. 9 displays Versions 4-6 of the extruded product with protein rich Euglena flour, pea protein and maskers. A displays version 4 formulation, B is version 5 and C is version 6 of the extruded product.

### DETAILED DESCRIPTION:

As a civilization we face significant challenges in the years ahead. The population growth predicted over the next few decades, about 9.7 billion global population by 2050, will cause severe food shortages. Malnutrition is a leading cause of death, accounting for about 3.5 million deaths per year. Global deforestation will cause the loss of a significant portion of the food we rely on currently, i.e. palm oil. The resources we utilize currently are unsustainable, two planets worth of resources will be needed to support the expected population by 2050. Accordingly, there is a significant need to identify sustainable alternatives. For example, food sources that can provide improved functionality, higher nutritional value, minimal waste stream, reduced water usages and reduced carbon dioxide emissions.

Microalgae are a rich source of protein, dietary fiber, essential fatty acids, vitamins, and minerals. After lipid removal, the residual biomass contains even higher concentrations of protein and other nutrients. Microalgae are good sources of long chain polyunsaturated fatty acids (“PUFA”) and have been used to enrich diets with omega-3 PUFA. Described herein are, inter alia, novel techniques for extracting a variety of components from heterotrophically cultivated microalgae, e.g., Euglena, without the use of harsh chemicals or solvents.

A specific species of algae named Euglena gracilis (hereinafter Euglena) belongs to a group of single-celled microalgae, that is often used as a candidate species for laboratory studies and technological applications. Euglena possess the representative features typical of eukaryotic cells such as a mitochondria, nucleus, and lysosome. Euglena can further be characterized for its long flagellum and large red eyespot. They are distinctive as they can produce their own nourishment (autotrophic) similar to plants, as well as eat and digest external food sources (heterotrophic) like animals. Euglena is a demonstrated, multifaceted model organism for study. Through optimizing the natural ability to employ singly or both modes of nourishment, Euglena can be directed to produce target compounds by adjusting key operating parameters in the production process. These critical adjustments can be used to enhance the natural mechanisms of the microorganism, to encourage rapid growth and the efficient conversion into valuable products with little waste production.

There are various methods, which are used to extract lipids, proteins and carbohydrates from a cell. In terms of extracting lipids from microalgae some methods include organic solvent extraction (with/without Soxhlet apparatus), supercritical fluid extraction, ultrasound or microwave assisted extraction, mechanical (i.e. pressing, milling), osmotic shock or enzyme based extraction. Extraction processes need to be optimized for microalgae due to the composition of their cellular make-up. However, processes that use toxic solvents (such as hexane) defeat the purpose of extracting the material in the safest and most environmentally friendly way. It can leave solvent residues in the product as well, which need to be removed before consumer consumption. In terms of soluble protein extraction, mechanical (bead milling), pH, temperature or enzymatic digestion has been used to break the microalgae cells, followed by purification of the released proteins.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Where a range of values is provided, it is intended that each intervening value between the upper and lower limit of that range and any other stated or intervening value in that stated range is encompassed within the disclosure. For example, if a range of 1 ml to 8 ml is stated, it is intended that 2 ml, 3 ml, 4 ml, 5 ml, 6 ml, and 7 ml are also explicitly disclosed, as well as the range of values greater than or equal to 1 ml and the range of values less than or equal to 8 ml.

The singular forms “a,” “an,” and “the” include plural referents unless the context clearly dictates otherwise. Thus, for example, reference to a “cell” includes a single cell as well as two or more of the same or different cells.

The word “about” when immediately preceding a numerical value means a range of plus or minus 5% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The term “batch” culturing refers to culturing wherein cells are allowed to consume all of the media until growth stops, typically about 2 days.

“Baked good” means a food item, typically found in a bakery, that is prepared by using an oven and usually contain a leavening agent. Baked goods include, but are not limited to brownies, cookies, pies, cakes and pastries.

“Bioreactor” and “fermenter” mean an enclosure or partial enclosure, such as a fermentation tank or vessel, in which cells are cultured typically in suspension.

“Bread” means a food item that contains flour, liquid, and usually a leavening agent. Breads are usually prepared by baking in an oven, although other methods of cooking are also acceptable. The leavening agent can be chemical or organic/biological in nature. Typically, the organic leavening agent is yeast. In the case where the leavening agent is chemical in nature (such as baking powder and/or baking soda), these food products are referred to as “quick breads.” Crackers and other cracker-like products are examples of breads that do not contain a leavening agent.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

“Cooked product” means a food that has been heated, e.g., in an oven, for a period of time.

“Creamy salad dressing” means a salad dressing that is a stable dispersion with high viscosity and a slow pour-rate. Generally, creamy salad dressings are opaque.

The verbs “cultivate,” “culture,” and “ferment,” and variants thereof, mean the intentional fostering of growth and/or propagation of one or more cells, typically microalgae, by use of culture conditions. Intended conditions exclude the growth and/or propagation of microorganisms in nature (without direct human intervention). The term “cultivated”, and variants thereof, refer to the intentional fostering of growth (increases in cell size, cellular contents, and/or cellular activity) and/or propagation (increases in cell numbers via mitosis) of one or more cells by use of intended culture conditions. The combination of both growth and propagation may be termed proliferation. The one or more cells may be those of a microorganism, such as microalgae. Examples of intended conditions include the use of a defined medium (with known characteristics such as pH, ionic strength, and carbon source), specified temperature, oxygen tension, carbon dioxide levels, and growth in a bioreactor.

The noun “culture,” as used herein, refers to a composition comprising microalgal biomass and optionally a liquid (e.g. culture media or water). For example, a culture may refer to a composition of microalgal biomass; a culture may also refer to a composition of microalgal biomass and liquid media and/or water.

“Dispersion” refers to a distribution of particles more or less evenly throughout a medium, including a liquid or gas. One common form of dispersion is an emulsion made up of a mixture of two or more immiscible liquids such as oil and water.

“Dry weight” and “dry cell weight” mean weight determined in the relative absence of water. For example, reference to microalgal biomass as comprising a specified percentage of a particular component by dry weight means that the percentage is calculated based on the weight of the biomass after substantially all water has been removed.

As used in this disclosure, the terms “emulsifying,” “emulsion” or a derivative thereof refers to where a substance or food additive, such as paramylon, is present in a food composition or food product as a single-phase mixture where a two-phase system of oil and water would normally have existed. An emulsion thus refers to a kinetically stable mixture of two normally immiscible liquids. A common example is mayonnaise in which oil is dispersed in water. In some other foods, the water is dispersed in oil.

“Edible ingredient” means any substance or composition which is fit to be eaten. “Edible ingredients” include, without limitation, grains, fruits, vegetables, proteins, herbs, spices, carbohydrates, and fats.

“Finished food product” and “finished food ingredient” mean a food composition that is ready for packaging, use, or consumption. For example, a “finished food product” may have been cooked or the ingredients comprising the “finished food product” may have been mixed or otherwise integrated with one another. A “finished food ingredient” is typically used in combination with other ingredients to form a food product.

The term “functional food product” as used herein refers to a food product given an additional function by adding new ingredients or more of existing ingredients. For example, whey protein is added to a food product to provide texture, water holding capacity or nutritional support to a food product.

“Food,” “food composition,” “food product,” and “foodstuff” mean any composition intended to be or expected to be ingested by humans as a source of nutrition and/or calories. Food compositions are composed primarily of carbohydrates, fats, water and/or proteins and make up substantially all of a person's daily caloric intake. A “food composition” can have a weight minimum that is at least ten times the weight of a typical tablet or capsule (typical tablet/capsule weight ranges are from less than or equal to 100 mg up to 1500 mg). A “food composition” is not encapsulated or formulated in tablet dosage form.

The term “gelling,” “gelificate” or a derivative thereof as used herein refers to a food composition or a food product in a gelatinous form. A gelatinous form is created by incorporating solids and liquids into a uniform three dimensional, semi-solid structure. A gelatinous food product is considered a soft gel when its tensile strength is in the range of 500-1000 g/cm², as seen in, for example, jelly and jams, nut butters (e.g. just nuts versions), jelly-like products, and fondant. A gelatinous food product is considered a hard gel when its tensile strength is in the range of 1000-3000 g/cm², as seen in, for example, gummy candy, confectionary gels (i.e. cookie filling), fruit gel bars, and fruit snacks.

“Foamability” as used herein, refers to the ability of a material, such as a protein, to rapidly adsorb on the air-liquid interface during whipping or bubbling, and to form a cohesive viscoelastic film by way of intermolecular interactions.

As used herein, “formulated” compositions of the present invention mean compositions comprising (defatted) microalgae with, for example, suitable excipients, stabilizers, binders, etc., that help make a stable microalgae containing composition that is suitable for oral consumption either as a dietary or nutritional supplement or as a food additive. For example, the compositions of the present invention can be formulated into solid, powder, or liquid forms.

As used herein, “formulated as a food additive” means formulated into a solid form (e.g., powder, tablet, pellet, etc.) or a liquid form (e.g., suspension, emulsion, mixture, etc.) to facilitate adding the composition to food as a food additive. For example, it may be desirable to add the composition to food or liquid during manufacturing or it may be desirable for a consumer to add the composition at the time of preparing and/or consuming a snack or meal. The particular manner of formulation will, therefore, depend on the food the composition will be added to and the point at which it will be added.

“Current Good Manufacturing Practices” and “CGMP” mean those conditions established by regulations set forth at 21 CFR 110 (for human food) and 111 (for dietary supplements), or comparable regulatory schemes established in locales outside the United States. The U.S. regulations are promulgated by the U.S. Food and Drug Administration under the authority of the Federal Food, Drug, and Cosmetic Act to regulate manufacturers, processors, and packagers of food products and dietary supplements for human consumption. All of the processes described herein can be performed in accordance with CGMP or equivalent regulations. In the United States, CGMP regulations for manufacturing, packing, or holding human food are codified at 21 CFR 110. These provisions, as well as ancillary provisions referenced therein, are hereby incorporated by reference in their entirety for all purposes. CGMP conditions in the United States, and equivalent conditions in other jurisdictions, apply in determining whether a food is adulterated (the food has been manufactured under such conditions that it is unfit for food) or has been prepared, packed, or held under unsanitary conditions such that it may have become contaminated or otherwise may have been rendered injurious to health. CGMP conditions can include adhering to regulations governing: disease control; cleanliness and training of personnel; maintenance and sanitary operation of buildings and facilities; provision of adequate sanitary facilities and accommodations; design, construction, maintenance, and cleanliness of equipment and utensils; provision of appropriate quality control procedures to ensure all reasonable precautions are taken in receiving, inspecting, transporting, segregating, preparing, manufacturing, packaging, and storing food products according to adequate sanitation principles to prevent contamination from any source; and storage and transportation of finished food under conditions that will protect food against physical, chemical, or undesirable microbial contamination, as well as against deterioration of the food and the container.

“Growth” means an increase in cell size, total cellular contents, number by cellular division, and/or cell mass or weight of an individual cell, including increases in cell weight due to conversion of a fixed carbon source into intracellular components.

The term “heterotrophic” or “heterotrophic environment,” as used herein, refers to an organism, such as a microalgae or microorganism including Euglena, which is under conditions such that it obtains nutrients and energy substantially entirely from exogenous sources of organic carbon, such as carbohydrates, lipids, alcohols, carboxylic acids, sugar alcohols, proteins, or combinations thereof. For example, Euglena is a heterotroph where it is in an environment substantially free of light.

The term “phototrophic” or derivatives, as used herein, refers to an organism, such as a microorganism including Euglena, when it is under a condition that it can carry out photon capture to acquire energy. For example, when an organism is phototrophic, it carries out photosynthesis to produce energy.

“Homogenate” means biomass that has been physically disrupted. Homogenization is a fluid mechanical process that involves the subdivision of particles into smaller and more uniform sizes, forming a dispersion that may be subjected to further processing. Homogenization is used in treatment of several foods and dairy products to improve stability, shelf-life, digestion, and taste. “Homogenize” means to blend two or more substances into a homogenous or uniform mixture. In some embodiments, a homogenate is created. In other embodiments, the biomass is predominantly intact, but homogeneously distributed throughout the mixture.

As used herein, the term “hydrocolloid” refers to long chain polymers of either carbohydrates (i.e. polysaccharides) or proteins that form a viscous solution or gel in water.

“Increased lipid yield” means an increase in the lipid/oil productivity of a microalgal culture that can achieved by, for example, increasing the dry weight of cells per liter of culture, increasing the percentage of cells that contain lipid, and/or increasing the overall amount of lipid per liter of culture volume per unit time.

“Lysate” means a solution containing the contents of lysed cells.

“Lysis” means the breakage of the plasma membrane and optionally the cell wall of a microorganism sufficient to release at least some intracellular content, which is often achieved by mechanical or osmotic mechanisms that compromise its integrity.

“Lysing” means disrupting the cellular membrane and optionally the cell wall of a biological organism or cell sufficient to release at least some intracellular content.

“Microalgal biomass,” “algal biomass,” and “biomass” mean a material produced by growth and/or propagation of microalgal cells. Biomass may contain cells and/or intracellular contents as well as extracellular material. Extracellular material includes, but is not limited to, compounds secreted by a cell.

“Microalgal flour” is a dry, particulate composition, fit for human consumption, comprising cells of microalgae, e.g., Euglena.

“Microalgal oil” and “algal oil” mean any of the lipid components produced by microalgal cells, including triacylglycerols (“TAG”).

“Nutritional supplement” means a composition intended to supplement the diet by providing specific nutrients as opposed to bulk calories. A nutritional supplement may contain any one or more of the following ingredients: a vitamin, a mineral, an herb, an amino acid, an essential fatty acid, and other substances. Nutritional supplements are typically tableted or encapsulated. A single tableted or encapsulated nutritional supplement is typically ingested at a level no greater than 15 grams per day. Nutritional supplements can be provided in ready-to-mix sachets that can be mixed with food compositions, such as yogurt or a “smoothie,” to supplement the diet, and are typically ingested at a level of no more than 25 grams per day.

“Oil” means any triacylglyceride (or triglyceride), produced by organisms, including microalgae, other plants, and/or animals. “Oil,” as distinguished from “fat,” refers, unless otherwise indicated, to lipids that are generally liquid at ordinary room temperatures and pressures. For example, “oil” includes vegetable or seed oils derived from plants, including without limitation, an oil derived from soy, rapeseed, canola, palm, palm kernel, coconut, corn, olive, sunflower, cotton seed, cuphea, peanut, camelina sativa, mustard seed, cashew nut, oats, lupine, kenaf, calendula, hemp, coffee, linseed, hazelnut, euphorbia, pumpkin seed, coriander, camellia, sesame, safflower, rice, tung oil tree, cocoa, copra, opium poppy, castor beans, pecan, jojoba, jatropha, macadamia, Brazil nuts, and avocado, as well as combinations thereof.

“Pasteurization” means a process of heating which is intended to slow microbial growth in food products. Typically, pasteurization is performed at a high temperature (but below boiling) for a short amount of time. As described herein, pasteurization can not only reduce the number of undesired microbes in food products, but can also inactivate certain enzymes present in the food product.

“Predominantly intact cells” and “predominantly intact biomass” mean a population of cells that comprise more than 40, and often more than 75, 90, 91, 92, 93, 94, 95, 96, 97, 98, and 99% intact cells. “Intact,” in this context, means that the physical continuity of the cellular membrane and/or cell wall enclosing the intracellular components of the cell has not been disrupted in any manner that would release the intracellular components of the cell to an extent that exceeds the permeability of the cellular membrane in culture.

“Predominantly lysed” means a population of cells in which more than 50%, and typically more than 75 to 90%, of the cells have been disrupted such that the intracellular components of the cell are no longer completely enclosed within the cell membrane.

“Proliferation” means a combination of both growth and propagation.

“Propagation” means an increase in cell number via mitosis or other cell division.

The term “shaking” as used herein refers to the movement of a sample, in an up and down or side to side, rapid, forceful or jerky movement. This may be done manually, or mechanically.

The term “sludge” refers to a solid dispersed in a liquid, forming a uniform composition which is not free flowing. A composition referred to as sludge typically contains greater than 20% total solids.

The term “slurry” refers to a composition which is free flowing. A composition referred to as a slurry typically contains less than 20% total solids.

The term “solution” as used herein refers to a homogeneous mixture of a substance (solute) dispersed through a liquid medium (solvent) that cannot be separated by the forces of gravity alone.

The term “substantially free” as used herein refers to the complete or near complete lack of light or a component. For example, a composition that is “substantially free” of water would either completely lack water, or so nearly completely lack water that the effect would be the same as if it completely lacked water.

As used herein, the term “stability” and derivatives thereof refer to heat stability, freeze-thaw stability, light stability, emulsion stability, or storage stability. Heat stability is the ability of a product or material to retain the same properties after exposure to a high heat for a single set period of time or a cycling of exposure times. Freeze-thaw stability is the ability of a product or material to retain the same properties after being frozen and subsequently thawed, which can be cycled through a number of freeze thaw cycles. Light stability is the ability of a product or material to retain the same properties after exposure to a light, such as sunlight or indoor light for a single set period of time or a cycling of exposure times. Emulsion stability is the ability of a product or material to retain an emulsion and to prevent separating, over time. Further, the term “stabilizer” relates to a material that provides stability described herein when added to a product or another material. For example, a stabilizer may be an ingredient incorporated into a final food formulation which preserves the structure and sensory characteristics of a food product over time, which would not otherwise be maintained in the absence of the stabilizer.

The term “solubility” as used herein, refers to the maximum amount of a substance that is able to be completely dissolved in a solution, usually in a specific amount.

“Suitable for human consumption” means a composition can be consumed by humans as dietary intake without ill health effects and can provide significant caloric intake due to uptake of digested material in the gastrointestinal tract.

“Uncooked product” means a composition that has not been subjected to heating but may include one or more components previously subjected to heating.

“V/V” or “v/v,” in reference to proportions by volume, means the ratio of the volume of one substance in a composition to the total volume of the composition. For example, reference to a composition that comprises 5% v/v microalgal oil means that 5% of the composition's volume is composed of microalgal oil (e.g., such a composition having a volume of 100 mm³ would contain 5 mm³ of microalgal oil), and the remainder of the volume of the composition (e.g., 95 mm³ in the example) is composed of other ingredients.

As used herein, the term “viscosity” refers to the resistance of a fluid when attempting to flow, may also be thought of as a measure of fluid friction.

“W/W” or “w/w,” in reference to proportions by weight, means the ratio of the weight of one substance in a composition to the weight of the composition. For example, reference to a composition that comprises 5% w/w microalgal biomass means that 5% of the composition's weight is composed of microalgal biomass (e.g., such a composition having a weight of 100 mg would contain 5 mg of microalgal biomass) and the remainder of the weight of the composition (e.g., 95 mg in the example) is composed of other ingredients.

“W/V” or “w/v” means the ratio of the weight of one substance in a composition to total volume of the composition. For example, reference to a composition that comprises 5% w/v microalgal biomass means that 5 g of microalgal biomass is dissolved in a final volume of 100 mL aqueous solution.

The term “whipping” as used herein, refers to the action of using a whisk, or a mixer to beat a sample in order to rapidly incorporate air and produce expansion.

The term “water holding capacity” or WHC or a derivative thereof as used herein relating to food composition or product refers to the ability to hold the food's own and added water during the application of forces, pressing, centrifugation, or heating. WHC may also be described as a physical property, for example, the ability of a food structure to prevent water from being released from the three-dimensional structure of, for example, a gel.

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Methods for Preparing Microalgal Biomass

The embodiments described herein provide methods for preparing microalgal biomass suitable for human consumption that is rich in nutrients, including lipid and/or protein constituents, methods of combining the same with edible ingredients and food compositions containing the same. Microalgal biomass can be prepared with a high protein content and/or with excellent functionality, and the resulting biomass incorporated into food products in which the oil and/or protein content of the biomass can substitute in whole or in part for oils and/or fats and/or proteins present in conventional food products, providing all the essential amino acids in a highly digestible form. Microalgal oil, which can comprise predominantly monounsaturated oil, provides health benefits compared with saturated, hydrogenated (trans fats) and polyunsaturated fats often found in conventional food products. The microalgal biomass also provides several beneficial micro-nutrients in addition to the oil and/or protein, such as algal-derived dietary fibers (both soluble and insoluble carbohydrates), phospholipids, glycoprotein, phytosterols, tocopherols, tocotrienols, and selenium. In embodiments described herein, the microalgal biomass is in the form of a solid, powder, or liquid formulated for oral administration. In embodiments described herein, the microalgal biomass is formulated as a food additive. In embodiments described herein, the microalgal biomass is processed into a microalgal flour. In embodiments described herein, a protein concentrate is extracted from the algal biomass. In embodiments described herein, a protein isolate is extracted from the microalgal biomass.

Media and Culture Conditions for Microalgae

Microalgae are cultured in liquid media to propagate microalgal biomass in accordance with the methods of the invention. In the methods of the invention, microalgal species are heterotrophically grown in a culture medium containing one or more carbon source, one or more nitrogen source, and one or more salt; maintaining a pH of between about 2.0 to about 4.0, in the absence of light. For some species of microalgae, for example, heterotrophic growth for extended periods of time such as 10 to 15 or more days under limited nitrogen conditions results in accumulation of high lipid content in cells. The protein cell content can be altered by the carbon:nitrogen (C:N) ratio of the growth media. High C:N ratio favors beta-glucan or carbohydrate biosynthesis. Lower C:N ratio favors protein accumulation. At production scale, the carbon and nitrogen are fed separately into the tank to control the C:N ratio. Ranges include about 6:1 to about 80:1 C:N ratio. Ratios of 7.2:1, 7.34:1, and 9.56:1 produce higher percentages of protein in biomass (from about 32% to about 49%), whereas ratios of 12:1, 20:1, 40:1, and 80:1 produce lower levels of protein (from about 16.7% to about 38.4%).

In embodiments, the microalgae may be replaced by an alga species selected from Euglena gracilis, Euglena sanguinea, Euglena deses, Euglena mutabilis, Euglena acus, Euglena viridis, Euglena anabaena, Euglena geniculata, Euglena oxyuris, Euglena proxima, Euglena tripteris, Euglena chlamydophora, Euglena splendens, Euglena texta, Euglena intermedia, Euglena polymorpha, Euglena ehrenbergii, Euglena adhaerens, Euglena clara, Euglena elongata, Euglena elastica, Euglena oblonga, Euglena pisciformis, Euglena cantabrica, Euglena granulata, Euglena obtusa, Euglena limnophila, Euglena hemichromata, Euglena variabilis, Euglena caudata, Euglena minima, Euglena communis, Euglena magnifica, Euglena terricola, Euglena velata, Euglena repulsans, Euglena clavata, Euglena lata, Euglena tuberculata, Euglena contabrica, Euglena ascusformis, Euglena ostendensis, Chlorella autotrophica, Chlorella coloniales, Chlorella lewinii, Chlorella minutissima, Chlorella pituita, Chlorella pulchelloides, Chlorella pyrenoidosa, Chlorella rotunda, Chlorella singularis, Chlorella sorokiniana, Chlorella variabilis, Chlorella volutis, Chlorella vulgaris, Schizochytrium aggregatum, Schizochytrium limacinum, Schizochytrium minutum, and combinations thereof. In certain embodiments, the microalgae is Euglena gracilis.

Embodiments described herein are directed to methods of heterotrophically culturing microalgae utilizing culture media containing a combination of carbon sources, nitrogen sources, and salts. Described culture media utilize all of microalgae's metabolic potential, including both aerobic and anaerobic metabolism. The combination of an oil, a sugar, an alcohol, an organic nitrogen, and an inorganic nitrogen source leads to higher conversion of input to output and faster growth of the microorganism.

In some embodiments growth media may also include exogenous nutrients and/or additives such as carbohydrates, lipids, alcohols, carboxylic acids, sugar alcohols, proteins, nitrogen, metals, vitamins, minerals, or combinations thereof

In embodiments, the carbon source is selected from an oil, a sugar, or an alcohol, carboxylic acids, potato liquor, ferulic acid, and combinations thereof. In embodiments the oil is an oil derived from soy, rapeseed, canola, palm, palm kernel, coconut, corn, olive, sunflower, cotton seed, cuphea, peanut, camelina sativa, mustard seed, cashew nut, oats, lupine, kenaf, calendula, hemp, coffee, linseed, hazelnut, euphorbia, pumpkin seed, coriander, camellia, sesame, safflower, rice, tung oil tree, cocoa, copra, opium poppy, castor beans, pecan, jojoba, jatropha, macadamia, Brazil nuts, or avocado, as well as combinations thereof. In one embodiment, the oil is canola oil, vegetable oil, soybean oil, coconut oil, olive oil, peanut oil, fish oil, avocado oil, palm oil, flax oil, corn oil, cottonseed oil, canola oil, rapeseed oil, sunflower oil, sesame oil, grapeseed oil, safflower oil, rice bran oil, propionate, and combinations thereof. The sugar may be selected from glucose, fructose, galactose, lactose, maltose, sucrose, molasses, glycerol, xylose, dextrose, honey, corn syrup, and combinations thereof. The alcohol may be selected from ethanol, methanol, isopropanol, and combinations thereof. In certain embodiments, the carbon source is glucose. The carboxylic acid may be selected from citric acid, citrate, fumaric acid, fumarate, malic acid, malate, pyruvic acid, pyruvate, succinic acid, succinate, acetic acid, acetate, lactic acid, lactate, and combinations thereof

In embodiments, the concentration of the carbon source is at a concentration of about 5 g/L to about 50 g/L, about 10 g/L to about 45 g/L, about 15 g/L to about 40 g/L, about 20 g/L to about 35 g/L, about 5 g/L to about 20 g/L, about 5 g/L to about 15 g/L, about 5 g/L to about 10 g/L. In embodiments, the concentration of the carbon source is at a concentration of about 15 g/L. In embodiments, the concentration of the carbon source is at a concentration of about 10 g/L.

In embodiments, the nitrogen source is selected from yeast extract, ammonium sulfate, glycine, urea, alanine, asparagine, corn steep, liver extract, lab lemco, peptone, skimmed milk, soy milk, tryptone, beef extract, tricine, plant source peptone, pea protein, brown rice protein, soybean peptone, mono-sodium glutamate (MSG), aspartic acid, arginine, potato liquor and combinations thereof. In certain embodiments, the nitrogen source is yeast extract. In certain embodiments, the nitrogen source is ammonium sulfate. In certain embodiments, the nitrogen source is a combination of yeast extract and ammonium sulfate.

In embodiments, the concentration of the nitrogen source is at a concentration of about lg/L to about 15 g/L, about 1.5 g/L to about 12.5 g/L, about 2 g/L to about 10 g/L, about 2.5 g/L to about 8.5 g/L, about 3 g/L to about 8 g/L, about 3.5 g/L to about 7.5 g/L, about 4 g/L to about 7 g/L about 4.5 g/L to about 6.5 g/L, or about 5 g/L to about 6 g/L. In embodiments, the concentration of the nitrogen source is at a concentration of about 10 g/L. In embodiments, the concentration of the nitrogen source is at a concentration of about 5 g/L. In embodiments, the concentration of the nitrogen source is at a concentration of about 2 g/L.

In embodiments, the salt is selected from ammonium nitrate, sodium nitrate, monopotassium phosphate, magnesium sulfate, magnesium sulfate heptahydrate, calcium chloride, calcium chloride dihydrate, calcium sulfate, calcium sulfate dihydrate, calcium carbonate, diammonium phosphate, dipotassium phosphate, and combinations thereof. In certain embodiments, the salt is monopotassium phosphate, magnesium sulfate, calcium chloride.

In embodiments, the concentration of the salt source is at a concentration of about 0.01 g/L to about 5.0 g/L, about 0.1 g/L to about 4.5 g/L, about 1.0 g/L to about 4.0 g/L, about 1.5 g/L to about 3.5 g/L, or about 2.0 g/L to about 3.0 g/L. In embodiments, the concentration of the salt source is at a concentration of about 0.1 g/L. In embodiments, the concentration of the salt source is at a concentration of about 1.0 g/L.

In embodiments, the culture media further comprises a metal. The metal is selected from iron (III) chloride, iron (III) sulfate, ammonium ferrous sulfate, ferric ammonium sulfate, manganese chloride, manganese sulfate, zinc sulphate, cobalt chloride, sodium molybdate, zinc chloride, boric acid, copper chloride, copper sulfate, ammonium heptamolybdate, and combinations thereof.

In embodiments, the culture media further comprises a vitamin mixture. The vitamin mixture contains a combination of the following: biotin (vitamin B7), thiamine (vitamin B1), riboflavin (vitamin B2), niacin (vitamin B3), pantothenic acid (vitamin B5), Pyridoxine (vitamin B6), Cyanocobalamin (vitamin B12), vitamin C, vitamin D, folic acid, vitamin A, vitamin B12, vitamin E, vitamin K, and combinations thereof

Cells and/or products produced by the methods of culturing microalgae described herein are collected at lag, exponential/log, stationary, or death phase. In an embodiment, cells and/or products produced are harvested or collected at lag, exponential, stationary, or death phase. In another embodiment, cells and/or products produced are harvested or collected at lag phase. In another embodiment, cells and/or products produced are harvested or collected at log phase. In another embodiment, cells and/or products produced are harvested or collected at stationary phase. In another embodiment, cells and/or products produced are harvested or collected at death phase.

When a microalgae culture reaches into the stationary phase, the concentration of the microorganisms in a culture reaches saturation. Saturation is determined by a number of measurements, including optical density, wet cell weight, dry cell weight, cell numbers, and/or time.

In an embodiment, the microalgae is grown to saturation measured as optical density at about 600 nm, wet cell weight, dry cell weight, or cell number. In an embodiment, saturation as measured by the optical density is about 2 to about 10. In an embodiment, saturation as measured by the wet cell weight is about 10 g/L to about 100 g/L. In an embodiment, saturation as measured by the dry cell weight is about 2 g/L to about 50 g/L. In an embodiment, saturation as measured by the cell number is about 2.0×10⁶ to about 10.0×10⁷ cells/mL. In an embodiment, the microorganism is grown for about 48 to about 350 hours, or up to about 75 days.

In general, cell culturing can be categorized into four culturing styles: batch, fed-batch, semi-continuous and continuous culture. In batch culturing, a large volume of nutrients (media) is added to a population of cells. The cells are then grown until the inputs in the media are depleted, the desired concentration of cells is reached, and/or the desired product is produced. At this point the cells are harvested and the process is repeated. In fed-batch culturing, media is added either at a constant rate or components are added in as needed to maintain the cell population. Once it has reached a maximum or product formation is reached, the majority of the cells are harvested and the remaining cells are then used to start the next cycle. Fed-batch is when growth fermenter is not yet full, the media is fed in to bring the culture to a target density. Once full, and at target density, continuous harvesting begins, the goal of which is maintaining a full, target density culture. During semi-continuous culture, a sample of fixed volume is removed at regular time intervals to make measurements and/or harvest culture components, and an equal volume of fresh media is immediately added to the culture, thereby instantaneously enhancing nutrient concentrations and diluting cell concentration. In a continuous culture, the cells are cultured in media under conditions in which additions to and removals from the media can be made over an extended period of time. As such, nutrients, growth factors and space are not exhausted.

In an embodiment, the method of heterotrophically culturing a microorganism is batch, fed-batch, semi-continuous or continuous. In another embodiment, the method of heterotrophically culturing a microorganism is batch. In another embodiment, the method of heterotrophically culturing a microorganism is fed-batch. In another embodiment, the method of heterotrophically culturing a microorganism is semi-continuous. In another embodiment, the method of heterotrophically culturing a microorganism is continuous.

In semi-continuous and continuous culture, fresh media is fed and culture is removed from the culture vessel. The culture can be removed at lag, exponential or stationary phase. In an embodiment, culture is removed from the culture vessel at lag, exponential or stationary phase. In another embodiment, culture is removed from the culture vessel at lag phase. In another embodiment, culture is removed from the culture vessel at exponential phase. In another embodiment, culture is removed from the culture vessel at stationary phase.

In semi-continuous and continuous culture, culture can also be removed from the culture vessel based on time interval. In an embodiment, the culture is removed at about, or at least 1, 2, 3, 4, 5, 6, 7, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188 or 192h from the beginning of the culture, or cycle of culture, or from a prior media addition. In semi-continuous and continuous culture, a cycle is defined as the turnover of the tank or bioreactor. Different parameters for growth are monitored and controlled for in the tank or bioreactor. These include the temperature, pH, dissolved oxygen level and agitation. A bioreactor or tank can be, e.g., 3 L to 20,000 L. For example, a bioreactor or tank may be 3 L-8 L, 36 L, 100 L and up to 20,000 L. Larger tanks are also possible such as 100,000 L or more. In an embodiment, the tank is at least 100 L, 1,000 L, 10,000 L, or 100,000 L. In another embodiment, the tank is up to 10,000 L, 100,000 L, 200,000 L, 500,000 L, or 1,000,000 L. A turnover is defined as the emptying of a vessel of one liquid such as a first media and the filling of the vessel by a second liquid such as a second media. With each subsequent emptying and filling that would represent another turnover. For example, a turnover of 2, turning over twice, or turns over 2 times would indicate that the tank was emptied and filled twice. During continuous culturing, there is substantially equal removal of culture and addition of source media. One turnover in continuous culturing would be when the volume of the vessel has been removed and replenished in vessel. In an embodiment, the method is semi-continuous or continuous culture in a tank or a bioreactor. In another embodiment, the method is semi-continuous or continuous culture in a tank up to 10,000 L, 100,000 L, 200,000 L, 500,000 L or 1,000,000 L. In another embodiment, the method is semi-continuous or continuous culture in a bioreactor up to 3 L, 5 L, 8 L, 10 L, 20 L, 30 L, 35 L, 36L, 40 L, or 50 L. In another embodiment, the media turns over 1, 2, 3, or 4 times a day in a tank or a bioreactor. In another embodiment, the media turns over up to 300 times in 75 days. In another embodiment, the media turns over at least 75, 150, 225, or 300 times in 75 days. In another embodiment, the method is continuous culture in a tank or a bioreactor, and the microorganism is grown for up to about 75 days. In another embodiment, the method is continuous culture in a tank or a bioreactor, the microorganism is grown for up to about 75 days, and the media turns over 300 times. In a specific embodiment, the method is continuous culture in a tank, the microorganism Euglena gracilis is grown for up to about 75 days, the media turns over 300 times.

In fed-batch, semi-continuous and continuous culture, media is added to the culture. The media can be added at lag, exponential or stationary phase. In an embodiment, media is added to the culture at lag, exponential or stationary phase. In another embodiment, media is added to the culture at lag phase. In another embodiment, media is added to the culture at exponential phase. In another embodiment, media is added to the culture at stationary phase.

In fed-batch, semi-continuous and continuous culture, media can also be added to the culture based on time interval. In an embodiment, the media is added at about, or at least 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188 or 192h from beginning of the culture, or cycle of culture, or from a prior media removal. In another embodiment, the media is added at about, or at most 10 min, 15 min, 30 min, 45 min, 60 min, 90 min, 2 h, 3 h, 4 h, 5 h, 6 h, 7 h, or 8 h from beginning of the culture, or cycle of culture, or from a prior media removal. In another embodiment, the media is added at approximately the same as the media is removed by the culture. Such additional media can be culture media, feed media, recycled culture media, spent media, supplemented media, and combinations thereof.

Culture media (also known as growth media) is a media with components needed in order to grow or culture the cells as described herein. Feed media is a media with components that is added to a culture in order to replenish nutrients. Feed media is at a working concentration or a concentrated level of components to limit dilution of the culture. Feed media is a media with components that is added to a culture in order to replenish nutrients. Feed media is at a working concentration or a concentrated level of components to limit dilution of the culture. Spent media is a media that has been used for cell culture i.e. culture media that has a lower level of growth components in it then at the start of culturing.

A spent media is also determined by the content of carbohydrate in the media after being used for culturing cells. For instance, the spent media can contain total carbohydrate, individual carbohydrate (e.g., glucose), or any combination of individual carbohydrate components (e.g., glucose and maltose) that is less than about 50, 40, 30, 20, 15, 10, 8, 7, 6, 5, 4, 3, 2.5, 2, 1.5, 1, 0.5, 0.4, 0.3, 0.2, 0.1 g/L. The depletion of carbohydrate in the spent media can be expressed as a percentage of starting amount of carbohydrate at the beginning of a culture, or a culture cycle. In an embodiment, the spent media comprises total carbohydrate of less than about 15, 10, 9, 8, 7, 6, 5, 4, 3, 2, 1, 0.9, 0.8, 0.7, 0.6, 0.5, 0.4, 0.3, 0.2, 0.1, 0.05, 0.01, 0.005, 0.001% from amount at the beginning of culturing, or cycle of culturing. In addition to carbohydrate, carboxylic acid is another carbon that is utilized by microorganism. Useful carboxylic acid includes citric acid, citrate, fumaric acid, fumarate, malic acid, malate, pyruvic acid, pyruvate, succinic acid, succinate, acetic acid, acetate, lactic acid, and lactate. In one embodiment, the spent media, recycled culture media, or hybrid culture media comprises carboxylic acid of less than about 20, 10, 5, 4, 3, 2, 1, 0.5, 0.4, 0.3, 0.2, or 0.1 g/L.

Recycled culture media is spent media that is used to culture cells for another passage, cycle, or for culturing cells from a different culture, lot, or strains. Recycled culture media is obtained by separating the recycled culture media from a source culture media, wherein the source culture media is in a lag phase, an exponential phase, or a stationary phase. Recycled culture media could be solely spent media, or it could be mixed with culture media (fresh growth media), or supplemented with one or more components that are depleted in the spent media. Recycled culture media can be obtained by separating the recycled culture media from a source culture media, wherein the source culture media is in a lag phase, an exponential phase, or a stationary phase.

A hybrid culture media is a culture media that is mixed with a fresh media and a recycled culture media.

In embodiments, the microalgae are grown to increase the concentration of protein in the cell. The protein cell content can be altered by the C:N ratio of the growth media. High C:N ratio favors beta-glucan or carbohydrate biosynthesis. Lower C:N ratio favors protein accumulation. At production scale, the carbon and nitrogen are fed separately into the tank to control the C:N ratio. C:N ranges from 6:1 to about 80:1. C:N ratios of about 7.2:1, 7.34:1 and 9.56:1 produce higher percentages of protein in biomass (from about 32% to about 49%), whereas higher C:N ratios of about 12:1, 20:1, 40:1, and 80:1 produce lower levels of protein (from about 16.7% to about 38.4%). During lag phase of cell growth, removal of carbon before culturing can increase protein. pH plays a role in protein accumulation in Euglena. At acidic pH (from about 1 to about 5), protein concentration in Euglena is high. At neural pH (from about 5 to about 8), protein concentration is at an intermediary level, except for pH 7 where there is an increase in protein concentration. At basic pH (from about 9 to about 14), protein concentration is at its lowest.

High protein biomass from algae is an advantageous material for inclusion in food products. The methods of the invention can also provide biomass that has an amount of protein as measured by % of dry cell weight selected from the group consisting of about 20% to about 90%, about 25% to about 85%, about 30% to about 70%, about 35% to about 65%, from about 40% to about 60%, about 45% to about 55%, about 60% to about 90%, and about 60% to about 80%.

In one embodiment, the chlorophyll content of the biomass is less than 200 ppm. Heterotrophic growth results in relatively low chlorophyll content (as compared to phototrophic systems such as open ponds or closed photobioreactor systems). Reduced chlorophyll content generally improves organoleptic properties of microalgae and therefore allows more algal biomass to be incorporated into a food product. The reduced chlorophyll content found in heterotrophically grown microalgae also reduces the green color in the biomass as compared to phototrophically grown microalgae. Thus, the reduced chlorophyll content avoids an often undesired green coloring associated with food products containing phototrophically grown microalgae and allows for the incorporation or an increased incorporation of algal biomass into a food product.

Harvesting or Concentration of Microalgae after Fermentation

Microalgal cultures generated according to the methods described herein yield microalgal biomass in fermentation broth/media. To prepare the biomass for use as a food composition, the biomass is concentrated, or harvested, from the fermentation medium. At the point of harvesting the microalgal biomass from the fermentation medium, the biomass comprises predominantly intact cells suspended in an aqueous culture medium.

To concentrate the biomass, a dewatering step is performed. Dewatering refers to the separation of the biomass from fermentation broth or other liquid medium to create a sludge. In embodiments described herein, during dewatering, the culture medium may be removed from the biomass by a method selected from the group consisting of decanting, centrifugation, filtration, or combinations thereof. The process of dewatering concentrates the biomass to about 1% to about 15% or about 10% to about 30% solids. In an embodiment, the harvesting of microorganisms is completed by settling cells. At scale, microorganisms are left to settle in the bottom of the tank to separate cells from source media. The microorganisms are then removed from the bottom of the tank and the remaining spent media is left in the tank. The tank may then be supplemented with fresh growth media, recycled culture media, or a mixture thereof.

Harvesting of microorganisms can also be established by mechanical methods, such as centrifugation. Centrifugation involves the use of centrifugal force to separate mixtures. During centrifugation, the denser components of the mixture migrate away from the axis of the centrifuge, while the less dense components of the mixture migrate towards the axis. By increasing the effective gravitational force (i.e., by increasing the centrifugation speed, or the rotor arm length), more dense material, such as solids, separate from the less dense material, such as liquids, and so separate out according to density. Centrifugation process can be performed either in batch mode using floor model centrifuges or in continuous mode through continuous centrifuges. Centrifugation of biomass and broth or other aqueous solution forms a concentrated sludge comprising principally the microalgal cells.

Membrane filtration can also be used for dewatering. One example of membrane filtration that is suitable for the present invention is tangential flow filtration (TFF), also known as cross-flow filtration. Tangential flow filtration is a separation technique that uses membrane systems and flow force to separate solids from liquids based on the selective exclusion of particles larger than the nominal pore size of the filter element.

Dewatering can also be effected with mechanical pressure directly applied to the biomass to separate the liquid fermentation broth from the microalgal biomass sufficient to dewater the biomass but not to cause predominant lysis of cells. Mechanical pressure to dewater microalgal biomass can be applied using, for example, a belt filter press.

In embodiments described herein, the dewatered microalgal biomass consists of predominantly intact cells. In embodiments, the content of intact cells in the dewatered microalgal biomass is about 25% to about 99%, about 30% to about 99%, about 35% to about 99%, about 40% to about 99%, about 45% to about 99%, about 50% to about 99%, about 55% to about 99%, about 60% to about 99%, about 65% to about 99%, about 70% to about 99%, about 75% to about 99%, about 80% to about 99%, about 85% to about 99%, about 90% to about 99%, or about 95% to about 99%.

Harvest of microorganisms in a culture may be done in whole or in part. In an embodiment, harvested microorganisms in a culture are about 1, 2, 3, 4, 5, 10, 15, 20, 25, 30, 35, 40, 45, 50, 55, 60, 65, 70, 75, 80, 85, 90, 95 or 100% of total source culture.

After concentration, the dewatered microalgal biomass can be further processed, as described herein, to produce microalgal flour, a protein concentrate, or a protein isolate. Alternatively, the microalgal biomass can be temporarily refrigerated or frozen to be utilized or processed at a later time.

Microalgal biomass can be utilized in preparation of food products described herein. For example, any microalgal biomass described herein may be combined with other ingredients as described herein to form a food product. For example, a wet microalgal biomass (e.g., that has not been subjected to drying as described herein) may be used alone or in combination with other ingredients as or to prepare a food product as described herein.

Method of Preparing Microalgal Flour

In embodiments described herein, the method for preparing a microalgal flour comprises: culturing microalgae, concentrating the microalgae into a microalgal biomass sludge, washing the microalgal biomass sludge, adjusting the pH of the microalgal biomass sludge, and drying the microalgal biomass sludge to produce the microalgal flour. In some embodiments, the microalgae is Euglena.

In embodiments described herein, the method for preparing a microalgal flour comprises culturing the microalgae, concentrating the microalgae into a microalgal biomass sludge, and washing the microalgal biomass sludge. In embodiments, the microalgal biomass sludge is washed about 1 to about 3 times, optionally about 2 times. In embodiments, the microalgal biomass sludge is washed with about 1 to about 4 times the volume of sludge, optionally about 2.5 times.

In embodiments described herein, the method for preparing a microalgal flour further comprises adjusting the pH of the microalgal biomass sludge. In embodiments, the pH of microalgal biomass sludge is adjusted to about 9 to about 11, about 7 to about 10, or about 6.5 to about 7.5. In embodiments, the pH of the microalgal biomass sludge is adjusted to about 7.

In embodiments described herein, the microalgal biomass sludge is dried to produce a protein-rich flour. The microalgal biomass sludge consists of predominantly intact cells. In embodiments, the content of intact cells in the microalgal biomass sludge is about 25% to about 99%, about 30% to about 99%, about 35% to about 99%, about 40% to about 99%, about 45% to about 99%, about 50% to about 99%, about 55% to about 99%, about 60% to about 99%, about 65% to about 99%, about 70% to about 99%, about 75% to about 99%, about 80% to about 99%, about 85% to about 99%, about 90% to about 99%, or about 95% to about 99%.

In embodiments, the concentrated microalgal biomass sludge is drum dried to a flake form to produce algal flake. In another embodiment, the concentrated microalgal biomass sludge is spray dried or flash dried to form a powder containing predominantly intact cells to produce a protein-rich flour. Drum drying is a process in which wet material can be dried in the form of very thin film. The drying process utilizes a cylinder or drum, that is typically mounted on a horizontal axis, that can be rotated at varied, controlled speeds. The drum is heated internally by steam, which condenses on the inside surface thus creating the drying effect via transfer from the heat inside the drum, through the metal walls to the thin layer of material on the external surface. The internal pressure of the drum is typically in the range of about 200 to about 500 kPa, with the external drum temperature reaching about 120 to about 155° C. Once dried, the microalgal flour, flake or powder is removed from the drum's surface with the help of a blade. This process is typically used for the production of various food items such as, but not limited to, soups, instant mashed potatoes, pre-cooked cereals and low grade milk powders and other thin, powdered materials.

In embodiments, the microalgal flour, flake or powder is 15% or less, 10% or less, 5% or less, 2-6%, or 3-5% moisture by weight after drying.

In embodiments described herein, the amount of protein in the microalgal flour, flake or powder is selected from the group consisting of about 25% to about 55%, about 30% to about 50%, or about 35% to about 45% by dry weight.

Method of Preparing Protein Concentrate and Protein Isolate

In embodiments described herein, the method of making protein concentrate comprises: culturing microalgae, bringing the culture to about 1% to about 30% solids, optionally about 10% to about 30% solids, optionally about 5% to about 15% solids, to form a biomass, adjusting the biomass to a pH of about 3 to about 11, homogenizing the biomass, centrifuging the homogenate, and separating the homogenate into three or more layers, for example a pellet, an aqueous middle layer, and a top lipid enriched layer, wherein the aqueous middle layer contains the soluble protein. The pellet nay contain more than one layer which is physically separated from the aqueous and insoluble components. The soluble protein is optionally precipitated by addition of an acid to adjust the pH to about 3.5 to about 5.5, optionally about 4 to about 5, optionally incubated at around 22° C. for at least 1 hour, and centrifuged, yielding a heavy phase made of protein concentrate sludge (protein sludge), and a light phase containing acid soluble cellular material and unprecipitated protein, referred to as whey. Optionally the homogenate may also be centrifuged yielding the soluble protein directly as protein concentrate sludge as a component of the pellet. Optionally the pelleted protein concentrate sludge is physically separated from other components of the pellet. Optionally, the protein concentrate sludge is diluted with water, optionally an equal weight of water, to create a protein concentrate slurry. The pH of the protein concentrate sludge or protein concentrate slurry is adjusted to about 5.5 to about 8.5, optionally about 6 to about 8. The protein concentrate slurry may be spray dried. In some embodiments, the microalgae is Euglena.

In embodiments, the protein concentrate has a protein concentration of about 40% to about 85%, about 45% to about 80%, about 50% to about 75%, about 50%, about 70%, about 55% to about 65%, or about 70% by dry weight.

In embodiments, the pellet has a beta glucan concentration of about 80% to about 95%, optionally greater than 95%.

In embodiments, the protein concentrate is de-fatted using organic solvents, thereby increasing the protein content to greater than 80%. In embodiments, the organic solvent is selected from the group consisting of acetone, benzyl alcohol, 1,3-butylene glycol, carbon dioxide, castor oil, citric acid esters of mono- and di-glycerides, ethyl acetate, ethyl alcohol (ethanol), ethyl alcohol denatured with methanol, glycerol (glycerin), glyceryl diacetate, glyceryl triacetate (triacetin), glyceryl tributyrate (tributyrin), hexane, isopropyl alcohol (isopropanol), methyl alcohol (methanol), methyl ethyl ketone (2-butanone), methylene chloride (dichloro-methane), monoglycerides and diglycerides, monoglyceride citrate, 1,2-propylene glycol (1,2-propanediol), propylene glycol mono-esters and diesters of fat-forming fatty acids, triethyl citrate, and combinations thereof.

In embodiments described herein, the method of making protein isolate comprises: culturing microalgae, bringing the culture to about 1% to about 30% solids, optionally about 10% to about 30% solids, optionally about 5% to about 15% solids, adjusting the culture to a pH of about 6 to about 11, homogenizing the culture, centrifuging the homogenate, and separating the homogenate into three layers, a pellet, a middle layer, and a top layer. The middle layer is precipitated by addition of acid to a pH of about 3.5 to about 5.5, optionally about 4 to about 5, optionally incubated at around 22° C. for at least 1 hour, and centrifuged, yielding a heavy phase made of protein concentrate sludge (protein sludge), and a light phase containing acid soluble cellular material and unprecipitated protein, referred to as whey. The precipitated protein concentrate sludge may be washed and filtered to further increase the protein content in the protein isolate. Optionally, the protein concentrate sludge is diluted with water, optionally an equal weight of water, to create a protein slurry. Optionally, the protein slurry is (a) centrifuged and (b) resuspended in water, where (a) and (b) are optionally repeated one or more times. The pH of the protein sludge or slurry is adjusted to about 5.5 to about 8.5, optionally about 6 to about 8. The protein slurry may be spray dried. In some embodiments, the microalgae is Euglena.

In embodiments, the protein isolate is prepared from protein concentrate sludge or powder. Optionally, the protein isolate is prepared by extracting lipids from the protein concentrate using a solvent. Optionally the solvent is selected from the group consisting of acetone, benzyl alcohol, 1,3-butlyene glycol, carbon dioxide, castor oil, ethyl acetate, ethyl alcohol, glycerol, glyceryl diacetate, glyceryl tributyrate, hexane, isopropyl alcohol, methyl alcohol, methyl ethyl ketone, methylene chloride, 2-nitropropane, 1,2-propylene glycol, propylene glycol mono- and di-esters, triethyl citrate, and combinations thereof. Optionally, the solvent used is hexane, ethanol or isopropanol. Optionally the solvent used is isopropanol. Optionally the solvent is removed from the protein isolate.

In embodiments, the protein isolate has a protein concentration of at least 40%, at least 80%, at least 85%, at least 90%, or at least 95% by dry weight.

In embodiments, the pellet has a beta glucan concentration of about 80% to about 95%, optionally greater than 95%.

In embodiments described herein, the method for preparing a protein concentrate or a protein isolate comprises culturing the microalgae as described herein.

In embodiments described herein, bringing the culture to a percent solids may include, for example, reconstituting previously cultured microalgal biomass (e.g., Euglena). In some embodiments, reconstituting previously cultured microalgal biomass includes resuspending previously cultured and dried microalgal biomass in liquid, for example water. In some embodiments, reconstituting previously cultured mciroalgal biomass includes thawing previously cultured and frozen biomass, and optionally resuspending the thawed biomass in liquid, for example water,

In embodiments described herein, the microalgal biomass is resuspended in an amount of water to bring the biomass concentration to an amount selected from the group consisting of about 5% to about 15%, about 3% to about 8%, about 3.5% to about 7.5%, about 4% to about 7%, about 4.5% to about 6.5%, or about 5% solids.

In embodiments described herein, bringing the culture to a percent solids may include, for example, concentrating or diluting a freshly cultured microalgae (e.g., Euglena) with a liquid (e.g., water).

In embodiments described herein, the pH of the culture is adjusted to an amount selected from the group consisting of about 6 to about 11, about 7 to about 10, about 6.5 to about 7.5, about 7, about 8, about 9, about 9.5, about 10, about 10.5, about 11, or about 11.5. In certain embodiments, the pH is adjusted with sodium hydroxide.

In embodiments described herein, the culture is homogenized. In certain embodiments, the homogenization is performed at about 10,000 psi to about 15,000 psi, about 10,500 psi to about 14,500 psi, about 11,000 psi to about 14,000 psi, about 11,500 psi to about 13,500 psi, about 12,000 psi to about 13,000 psi, or about 12,500 psi. In certain embodiments the homogenization is performed at about 2,000 to about 5,000 psi, about 2,000 psi to about 2,500 psi, about 3,000 psi to about 3,500 psi, about 4,000 psi to about 4,500 psi, or about 5,000 psi to about 5,500 psi. In certain embodiments, the homogenization is performed at much lower pressures, such as about 50 psi. In certain embodiments, the homogenization is performed with multiple passes.

In embodiments described herein, the homogenization or cell lysis is performed using one of the following mechanical techniques: high pressure homogenization, bead milling, high-shear mixing, French-press, ultrasonication, or use of chemical processes including but not limited to enzymatically induced lysis. Homogenization can be carried out in the presence of a liquid, for example, water, a solvent, or a buffered solvent. Homogenization can be carried out with a homogenizer, for example Polytron PTA-7 and OMNI GLH-01. The homogenizer can be a high pressure homogenizer. In some embodiments, the resuspended biomass is homogenized using a high pressure homogenizer at 12,500 psi at about 20° C. to about 27° C. Homogenization time is dependent on the equipment capacity, i.e. 24 L/h. The collected homogenate is centrifuged at 5,000 rpm for 5 min (or 3,500 rpm for 10 min) at about 20° C. to about 27° C.

In embodiments described herein, the homogenate is centrifuged using a bucket centrifuge at a speed selected from the group consisting of about 3,000 rpm to about 6,000 rpm, about 3,100 rpm to about 5,900 rpm, about 3,200 rpm to about 5,800 rpm, about 3,300 rpm to about 5,700 rpm, about 3,400 rpm to about 5,600 rpm, about 3,500 rpm to about 5,500 rpm, about 3,600 rpm to about 5,400 rpm, about 3,700 rpm to about 5,300 rpm, about 3,800 rpm to about 5,200 rpm, about 3,900 rpm to about 5,100 rpm, about 4,000 rpm to about 5,000 rpm, about 4,100 rpm to about 4,900 rpm, about 4,200 rpm to about 4,800 rpm, about 4,300 rpm to about 4,700 rpm, or about 4,400 rpm to about 4,600 rpm. In some embodiments, the centrifugation may be performed using decanters, disc-stack centrifuges, or spiral disc centrifuges.

In embodiments described herein centrifugation is carried out in a range in g force from about 250 times the force of gravity (×g) to about 16,000×g. In embodiments described herein centrifugation is carried out in a range of about 250×g to about 16,000×g, about 500×g to about 16,000×g, about 1000×g to about 16,000×g, about 1,000×g to about 15,000×g, about 1,000×g to about 14,000×g, about 1,000×g to about 13,000×g, about 1,000×g to about 12,000×g, about 1,000×g to about 11,000×g, about 1,000×g to about 10,000×g, about 1,000×g to about 9,000×g, about 1,000×g to about 8,000×g, about 1,000×g to about 7,000×g, about 1,000×g to about 6,000×g, about 1,000×g to about 5,000×g, about 1,000×g to about 4,000×g, about 1,000×g to about 3,000×g, or about 1,000×g to about 2,000×g.

In embodiments described herein, the homogenate is centrifuged for about 5 minutes to about 30 minutes, about 10 minutes to about 20 minutes, about 13 minutes, or about 15 minutes.

In embodiments described herein, the centrifuged homogenate will be separated into 3 layers: the pellet, the middle aqueous layer and the top lipid enriched layer. The pellet is the beta-glucan (paramylon), which has a white/beige color. The middle aqueous layer contains the soluble protein liquid, referred to as the protein skim. The top layer is oil in the form of a waxy emulsion. The 3 layers are separated and processed separately.

In embodiments described herein, the middle soluble protein liquid or protein skim is used to produce the protein concentrate and/or isolate.

In embodiments described herein, the pH of the protein skim is adjusted to about 3.5 to about 5.5, about 4 to about 5, about 3 to about 5, about 3.5, about 4, about 4.5, or about 5.

In embodiments described herein, the pH adjusted protein skim is incubated at 22° C. for at least 1 hour, about 1 hour to about 24 hours, about 1 hour to about 12 hours, about 1 hour to about 6 hours, about 1 hour to about 4 hours, or about 1 hour to about 2 hours. In embodiments, the pH adjusted protein skim is agitated during incubation.

In embodiments described herein, the incubated pH adjusted protein skim is centrifuged. In embodiments described herein, the centrifugation is at a speed selected from the group consisting of about 3,000 rpm to about 6,000 rpm, about 3,100 rpm to about 5,900 rpm, about 3,200 rpm to about 5,800 rpm, about 3,300 rpm to about 5,700 rpm, about 3,400 rpm to about 5,600 rpm, about 3,500 rpm to about 5,500 rpm, about 3,600 rpm to about 5,400 rpm, about 3,700 rpm to about 5,300 rpm, about 3,800 rpm to about 5,200 rpm, about 3,900 rpm to about 5,100 rpm, about 4,000 rpm to about 5,000 rpm, about 4,100 rpm to about 4,900 rpm, about 4,200 rpm to about 4,800 rpm, about 4,300 rpm to about 4,700 rpm, or about 4,400 rpm to about 4,600 rpm. In embodiments described herein, the centrifugation is for about 5 minutes to about 30 minutes, about 10 minutes to about 20 minutes, about 13 minutes, or about 15 minutes.

In embodiments described herein, the incubated pH adjusted protein skim is separated using filtration.

In embodiments described herein, the method of making protein concentrate further comprises that the protein sludge from the centrifugation step is saved and the supernatant is discarded.

In embodiments described herein, the method of making protein concentrate further comprises that the protein sludge is resuspended in an equal weight of water to produce a protein slurry.

In embodiments described herein, the method of making protein concentrate further comprises that the pH of the protein slurry be adjusted to an amount selected from the group consisting of about 5.5 to about 8.5, about 6 to about 8, about 6.5 to about 7.5, or about 7. In certain embodiments, the pH is adjusted using the appropriate acid or base. In certain embodiments, the acid is selected from the group consisting of hydrogen chloride, potassium chloride, acetic acid, adipic acid, carbonic acid, citric acid, fumaric acid, gluconic acid, hydrochloric acid, lactic acid, malic acid, metatartaric acid, phosphoric acid, sulphuric acid, tartaric acid, or any combination thereof. In certain embodiments, the base is selected from the group consisting of ammonium bicarbonate, ammonium carbonate, ammonium citrate, ammonium hydroxide, ammonium phosphate, calcium acetate, calcium carbonate, calcium hydroxide, calcium oxide, calcium phosphate, calcium sulphate, magnesium carbonate, magnesium hydroxide, magnesium oxide, magnesium phosphate, magnesium sulphate, potassium bicarbonate, potassium carbonate, potassium hydroxide, potassium phosphate, sodium bicarbonate, sodium carbonate, sodium hydroxide, sodium phosphate, or any combination thereof.

In embodiments described herein, the method of making protein concentrate further comprises spray drying the pH adjusted protein slurry. In embodiments described herein, the spray drying can be accomplished using a spray dryer outfitted with any of a high-pressure atomizer, two-fluid atomizer or centrifugal atomizer. Alternatively, the drying may be accomplished using freeze drying, drum drying or pulse-combustion drying.

Chemical Composition of Microalgal Biomass

The microalgal biomass generated by the culture methods described herein comprises microalgal oil and/or protein as well as other constituents generated by the microalgae or incorporated by the microalgae from the culture medium during fermentation.

In embodiments described herein, the present invention provides a microalgal flour, which is a whole-cell microalgal biomass containing predominantly intact cells, or a homogenate of microalgal biomass containing predominantly or completely lysed cells. In certain embodiments, the microalgal flour is in the form of a powder, wherein the microalgal biomass comprises at least 40% protein by dry weight and less than 20% of triglyceride (oil) by dry weight. In some embodiments, the microalgal biomass comprises at least 20% carbohydrate by dry weight. In some embodiments, the microalgal biomass comprises at least 10% dietary fiber by weight. In some embodiments, the protein is at least 30% to about 45% digestible crude protein.

In some embodiments, the average size of particles is about 100 μm to about 200 μm. In some embodiments, the average size of particles in the powder is 180 μm. In some embodiments, the average size of the particles is less than 100 μm. In some embodiments, the average size of particles in the powder is about 40 μm.

In some embodiments, the powder is formed by micronizing microalgal biomass to form an emulsion and drying the emulsion. In some embodiments, the microalgal flour has a moisture content of 10% or less by weight.

In some embodiments, the microalgal flour further comprises a food compatible preservative. In some embodiments, the microalgal flour further comprises a food compatible antioxidant.

In embodiments described herein, the composition provides a food ingredient comprising the microalgal flour discussed above combined with at least one other protein product that is suitable for human consumption, wherein the food ingredient contains at least 50% protein by dry weight. In some embodiments, the at least one other protein product is derived from a vegetarian source. In some cases, the vegetarian source is selected from the group consisting of soy, pea, bean, milk, whey, rice, lentil, faba bean, chickpea, and wheat.

In embodiments described herein, the composition provides a food composition formed by combining microalgal biomass comprising at least 40% protein by dry weight and less than 20% of triglyceride (oil) by dry weight and at least one other edible ingredient. In some embodiments, at least one other edible ingredient is a meat product. In some embodiments, the food composition is an uncooked product. In some embodiments, the food composition is a cooked product.

In embodiments described herein, the composition provides a food composition formed by combining microalgal biomass comprising at least 13% total dietary fiber by weight and at least one edible ingredient. In some embodiments, the microalgal biomass comprises between about 13% to about 35% total dietary fiber by weight. In some embodiments, the microalgal biomass comprises between about 4% to about 10% soluble fiber. In some embodiments, the microalgal biomass comprises between about 5% to about 25% insoluble fiber.

In some embodiments, the food product contains heterotrophically grown microalgae of reduced chlorophyll content compared to phototrophically grown microalgae.

In some embodiments the chlorophyll content of microalgal flour is less than 5 ppm, less than 2 ppm, or less than 1 ppm.

High protein microalgal biomass has been generated using different methods of culture. Microalgal biomass with a higher percentage of protein content is useful in accordance with the embodiments described herein. Microalgal biomass generated by culture methods described herein typically comprises at least 30% protein by dry cell weight. In some embodiments, the microalgal biomass comprises at least 40%, 50%, 75% or more protein by dry cell weight. In some embodiments, the microalgal biomass comprises from 30-75% protein by dry cell weight or from 40-60% protein by dry cell weight. In some embodiments, the protein in the microalgal biomass comprises at least 40% digestible crude protein. In some embodiments, the protein in the microalgal biomass comprises at least 50%, 60%, 70%, 80%, or at least 90% digestible crude protein. In some embodiments, the protein in the microalgal biomass comprises from 40-90% digestible crude protein, from 50-80% digestible crude protein, or from 60-75% digestible crude protein.

In some embodiments, the biomass comprises less than 0.01 mg/100 g selenium. In some embodiments, the biomass comprises about 20% to about 50% w/w algal polysaccharide. In some embodiments, the biomass comprises at least 15% w/w algal glycoprotein. In some embodiments, the biomass or oil derived from the biomass comprises between 0-200, 0-115, or 50-115 mcg/g total carotenoids, and in specific embodiments 20-70 or 50-60 mcg/g of the total carotenoid content is lutein. In some embodiments, the biomass comprises at least 0.5% to about 10% algal phospholipids. In some embodiments, the biomass or oil derived from the algal biomass contains at least 0.10, 0.02-0.5, or 0.05-0.3 mg/g total tocotrienols, and in specific embodiments 0.05-0.25 mg/g is alpha tocotrienol. In some embodiments, the biomass or oil derived from the algal biomass contains between 0.125 mg/g to 0.35 mg/g total tocotrienols. In some embodiments, the oil derived from the algal biomass contains at least 5.0, 1-8, 2-6 or 3-5 mg/100 g total tocopherols, such as Vitamin E, and in specific embodiments 2-6 mg/100 g is alpha tocopherol. In some embodiments, the oil derived from the algal biomass contains between 5.0 mg/100 g to 10 mg/100 g tocopherols. In some embodiments, the biomass contains between 100-1000 mg gamma aminobutyric acid (GABA).

In some embodiments, the microalgal biomass comprises 20-50% carbohydrate by dry weight. In other embodiments, the biomass comprises 25-40% or 30-35% carbohydrate by dry weight. Carbohydrate can be dietary fiber as well as free sugars such as sucrose and glucose. In some embodiments, the free sugar in microalgal biomass is 1-10%, 2-8%, or 3-6% by dry weight. In certain embodiments, the free sugar component comprises sucrose.

In some embodiments, the microalgal biomass comprises at least 10% soluble fiber. In some embodiments, the microalgal biomass comprises at least 20% to 25% soluble fiber. In some embodiments, the microalgal biomass comprises at least 30% insoluble fiber. In some embodiments, the microalgal biomass comprises at least 50% to at least 70% insoluble fiber. Total dietary fiber is the sum of soluble fiber and insoluble fiber. In some embodiments, the microalgal biomass comprises at least 40% total dietary fiber. In other embodiments, the microalgal biomass comprises at least 50%, 55%, 60%, 75%, 80%, 90%, to 95% total dietary fiber.

Processing Microalgal Biomass into Finished Food INGREDIENTS

The concentrated microalgal biomass produced in accordance with the methods described herein is itself a finished food ingredient and may be used in foodstuffs without further, or with only minimal, modification. For example, the microalgal biomass can be vacuum-packed or frozen. Alternatively, the microalgal biomass may be dried via lyophilization, a “freeze-drying” process, in which the biomass is frozen in a freeze-drying chamber to which a vacuum is applied. The application of a vacuum to the freeze-drying chamber results in sublimation (primary drying) and desorption (secondary drying) of the water from the biomass. However, the present disclosure provides a variety of microalgal derived finished food ingredients with enhanced properties.

The microalgal biomass, microalgal flour, protein concentrate, and protein isolate described herein provide improved functionality over traditional plant protein, the microalgal biomass does not increase the viscosity of the food products which it is added to or formulated with. In some embodiments, the food product has a viscosity of about 1 mPa·s to about 2000 mPa·s at 25° C.

The microalgal biomass, microalgal flour, protein concentrate, and protein isolate described herein has good foamability.

In some embodiments, the composition is formulated into an oral dosage form that is swallowable, chewable, or dissolvable. Swallowable compositions are well known in the art and are those that do not readily dissolve when placed in the mouth and may be swallowed whole without any chewing.

To prepare the swallowable compositions, the microalgal biomass (wet or dried) may be combined with a suitable carrier (e.g., excipients, stabilizers, binders, etc.)

according to conventional compounding techniques. In some embodiments, the swallowable composition may be coated with a polymeric film. Such a film coating has several beneficial effects. First, it reduces the adhesion of the compositions to the inner surface of the mouth, thereby increasing one's ability to swallow the compositions. Second, the film may aid in masking the unpleasant taste of certain ingredients. Third, the film coating may protect the composition of the present invention from atmospheric degradation. Polymeric films that may be used in preparing the swallowable compositions include vinyl polymers such as polyvinylpyrrolidone, polyvinyl alcohol, and acetate, cellulosics such as methyl and ethyl cellulose, hydroxyethyl cellulose and hydroxylpropyl methylcellulose, acrylates, and methacrylates, copolymers such as the vinyl-maleic acid and styrene-maleic acid types, and natural gums and resins such as zein, gelatin, shellac, and acacia.

Chewable compositions are those that have a palatable taste and mouth-feel, are relatively soft, and quickly break into smaller pieces and begin to dissolve after chewing such that they are swallowed substantially as a solution.

To create chewable compositions, certain ingredients should be included to achieve the attributes just described. For example, chewable compositions should include ingredients that create pleasant flavor and mouth-feel and promote relative softness and dissolvability in the mouth. The following discussion describes ingredients that may help to achieve these characteristics.

Chewable compositions should begin to break and dissolve in the mouth shortly after chewing begins such that the compositions can be swallowed substantially as a solution. The dissolution profile of chewable compositions may be enhanced by including rapidly water-soluble fillers and excipients. Rapidly water-soluble fillers and excipients preferably dissolve within about 60 seconds of being wetted with saliva. Indeed, it is contemplated that if enough water-soluble excipients are included in the compositions of the present invention, they may become dissolvable rather than chewable composition forms. Examples of rapidly water soluble fillers suitable for use with the present invention include, by way of example and without limitation, saccharides, amino acids, and the like. Disintegrants also may be included in the compositions of the present invention in order to facilitate dissolution. Disintegrants, including permeabilizing and wicking agents, are capable of drawing water or saliva up into the compositions which promotes dissolution from the inside as well as the outside of the compositions. Such disintegrants, permeabilizing and/or wicking agents that may be used in the present invention include, by way of example and without limitation, starches, such as corn starch, potato starch, pre-gelatinized, and modified starches thereof, cellulosic agents, such as Ac-di-sol, montrnorrilonite clays, cross-linked PVP, sweeteners, bentonite, microcrystalline cellulose, croscarmellose sodium, alginates, sodium starch glycolate, gums, such as agar, guar, locust bean, karaya, pectin, Arabic, xanthan and tragacanth, silica with a high affinity for aqueous solvents, such as colloidal silica, precipitated silica, maltodextrins, beta-cyclodextrins, polymers, such as carbopol, and cellulosic agents, such as hydroxymethylcellulose, hydroxypropylcellulose and hydroxyopropylmethylcellulose.

In embodiments described herein, the microalgal biomass composition may be formulated into the form of a liquid gelatin capsule. This may comprise the microalgal biomass suspended in, dissolved in, or contained in an appropriate liquid vehicle encapsulated in a gelatin shell generally comprising gelatin together with a plasticizer such as glycerin or sorbitol. The filler material may comprise, for example, polyethylene glycols.

Microalgal Flour

The protein content of microalgal flour can vary depending on the percent protein of the microalgal biomass. Microalgal flour can be produced from microalgal biomass of varying protein content. In certain embodiments, the microalgal flour is produced from microalgal biomass of the same protein content. In some embodiments, the microalgal flour is produced from microalgal biomass of different protein content. In the latter case, microalgal biomass of varying protein content can be combined and then the homogenization step performed. In other embodiments, microalgal flour of varying protein content is produced first and then blended together in various proportions in order to achieve a microalgal flour product that contains the final desired protein content. In certain embodiments, microalgal biomass of different protein profiles can be combined together and then homogenized to produce microalgal flour. In another embodiment, microalgal flour of different protein profiles is produced first and then blended together in various proportions in order to achieve an microalgal flour product that contains the final desired protein profile.

The microalgal flour described herein is useful for a wide range of food preparations. Because of the protein content, fiber content and the micronized particles, microalgal flour is a multifunctional food ingredient. Microalgal flour can be used in baked goods, quick breads, yeast dough products, egg products, dressing, sauces, nutritional beverages, algal milk, pasta and gluten free products. Gluten-free products can be made using microalgal flour and another gluten-free product such as amaranth flour, arrow root flour, buckwheat flour, rice flour, chickpea flour, cornmeal, maize flour, millet flour, potato flour, potato starch flour, quinoa flour, sorghum flour, soy flour, bean flour, legume flour, tapioca (cassava) flour, teff flour, artichoke flour, almond flour, acorn flour, coconut flour, chestnut flour, corn flour and taro flour. Microalgal flour, in combination with other gluten-free ingredients is useful in making gluten-free food products such as baked goods (cakes, cookie, brownies and cake-like products (e.g., muffins)), breads, cereal, crackers and pastas. Additional details of formulating these food products and more with microalgal flour is described in the Examples below.

Microalgal flour can be used in baked goods in place of convention protein sources (e.g., nuts, meat products, or beans) and eggs. Baked goods and gluten free products have superior moisture content and a crumb structure that is indistinguishable from conventional baked goods made with butter and eggs. Because of the superior moisture content, these baked goods have a longer shelf life and retain their original texture longer than conventional baked goods that are produced without microalgal flour.

The water activity (Aw) of a food can be an indicator of shelf-life retention in a prepared food product. Water activity (ranging from 0 to 1) is a measure of how efficiently the water present in a food product can take part in a chemical or physical reaction. The water activity of some common foods representing the spectrum of Aw are: fresh fruit/meat/milk (1.0-0.95); cheese (0.95-0.90); margarine (0.9-0.85); nuts (0.75-0.65); honey (0.65-0.60); salted meats (0.85-0.80); jam (0.8-7.5); pasta (0.5); cookies (0.3); and dried vegetables/crackers (0.2). Most bacteria will not grow at water activities below 0.91. Below 0.80 most molds cannot be grown and below 0.60 no microbiological growth is possible. By measuring water activity, it is possible to predict the potential sources of spoilage. Water activity can also play a significant role in determining the activity of enzymes and vitamins in foods, which can have a major impact in the food's color, taste and aroma.

Microalgal flour can also act as a protein supplement for use in smoothies, sauces, or dressings.

Microalgal flour can also be added to powdered or liquid eggs, which are typically served in a food service setting. The combination of a powdered egg product and microalgal flour is itself a powder, which can be combined with an edible liquid or other edible ingredient, typically followed by cooking to form a food product. In some embodiments, the microalgal flour can be combined with a liquid product that will then be sprayed dried to form a powdered food ingredient (e.g., powdered eggs, powdered sauce mix, powdered soup mix, etc). In such instances, it is advantageous to combine the microalgal flour after homogenization, but before drying so that is a slurry or dispersion, with the liquid product and then spray dry the combination, forming the powdered food ingredient. This co-drying process will increase the homogeneity of the powdered food ingredient as compared to mixing the dried forms of the two components together. The addition of microalgal flour improves the appearance, texture and mouth-feel of powdered and liquid eggs and also extends improved appearance, texture and mouth-feel over time, even when the prepared eggs are held on a steam table.

Microalgal flour can be used to formulate reconstituted food products by combining microalgal flour with one or more edible ingredients and liquid, such as water. The reconstituted food product can be a beverage, dressing (such as salad dressing), sauce (such as a cheese sauce), or an intermediate such as a dough that can then be baked. In some embodiments, the reconstituted food product is then subjected to shear forces such as pressure disruption or homogenization. A preferred microalgal flour particle size in a reconstituted food product is an average of 1 to 15 micrometers.

Combining Microalgal Biomass or Materials Deirved Therefrom with Other Food Ingredients

The compositions described herein containing microalgal biomass are formulated, e.g., for consumption as a dietary, nutritional, or food supplement. In some embodiments, a composition comprising microalgal biomass is in a solid, powder, or liquid form and is formulated for oral administration. In another aspect, a composition comprising microalgal biomass is formulated as a food additive.

In some embodiments, the composition is formulated as a food additive and added to food. For example, and without limitation, the composition may be added to sauce, tea, candy, cookies, cereals, breads, fruit mixes, fruit salads, salads, snack bars, protein bars, fruit leather, yogurt, health bars, granola, smoothies, soups, juices, cakes, pies, shakes, ice cream, protein beverages, nutritional beverage, nutritional beverage supplement, animal analogues, and health drinks. The composition formulated as a food additive may have any of the additives described supra for the oral dosage formulations.

In some embodiments, the composition is formulated as a non-dairy product, such as a cheese or yogurt.

In some embodiments, the composition is formulated as an animal analogue. In embodiments, the animal analogue is a meat analogue. In some embodiments, the animal analogue is an egg analogue. In some embodiments, the egg analogue is in liquid form. In some embodiments, the egg analogue is in dry or powdered form. In some embodiments, the animal analogue is a selected from the group consisting of a meat analogue, sausage analogue, pepperoni/cured meat analogue, chicken analogue, turkey analogue, pork analogue, bacon analogue, beef analogue, tofu replacement, ground beef analogue, jerky analogue, an egg analogue, egg replacement, hard-boiled egg replacement, powdered egg replacement, liquid egg replacement, frozen egg replacement, salad dressing, mayonnaise, and combinations thereof.

In some embodiments, the composition is formulated as an extruded product, such as a protein crisp.

In some embodiments, the composition is formulated as a nutritional beverage or nutritional beverage supplement. In some embodiments, the nutritional beverage is in liquid form. In some embodiments, the nutritional beverage is in powdered form.

In some embodiments, the composition is formulated as a protein bar.

Any of the compositions described herein can also be formulated as food products.

In some embodiments, the composition is in liquid form. In some embodiments, the composition is in powdered form.

In some embodiments, microalgal biomass is combined with other ingredients (e.g, maskers, flavorings, and/or other additional ingredients). Such other ingredients may be added at any point of culturing or processing the microalgal biomass or added directly to a final or intermediate food product. For example, such other ingredients may be applied during culturing, to wet biomass prior to drying (e.g., spray-drying), during drying (e.g., spray drying), to wet biomass, during spray drying, to spray dried materials, to the final food product, or any combination thereof. As discussed, herein, wet biomass may be used directly in preparation of food product. Accordingly, compositions and food products according to some embodiments comprise additional ingredients, including flavoring, masking agents and/or additional ingredients. Methods according to some embodiments involve applying a flavoring, masking agent, and/or additional ingredients to the culturing microalgae and/or microalgae biomass.

Typically, the compositions will contain from about 0.01% to about 100%, about 0.01% to about 99%, about 0.01% to about 95%, about 0.01% to about 80%, about 0.01% to about 75%, about 0.1% to about 100%, about 0.1% to about 99%, about 0.1% to about 95%, about 0.1% to about 80%, about 0.1% to about 75%, about 1% to about 100%, about 1% to about 99%, about 1% to about 95%, about 1% to about 80%, about 1% to about 75%, about 5% to about 100%, about 5% to about 99%, about 5% to about 95%, about 5% to about 80%, about 5% to about 75%, about 10% to about 100%, about 10% to about 99%, about 10% to about 95%, about 10% to about 80%, about 10% to about 75% microalgal biomass.

In some embodiments, the food composition comprising at least 0.1% w/w microalgal biomass and one or more other edible ingredients. In some embodiments, the food composition comprising the microalgal biomass at least 10% protein by dry weight. In some embodiments, the microalgal biomass contains about 25%, about 30%, about 35%, about 40%, about 45%, about 50%, about 55%, or 60% protein by dry weight. In some embodiments, the microalgal biomass contains 10-90%, 10-75%, 25-75%, 40-75% or 50-70% protein by dry weight. In preferred embodiments, the microalgal biomass is grown under heterotrophic conditions and has reduced green pigmentation.

In embodiments described herein, the food composition comprising at least 0.1% w/w microalgal biomass and one or more other edible ingredients, wherein the microalgal biomass comprises at least 30% protein by dry weight, at least 40% protein by dry weight, at least 45% protein by dry weight, at least 50% protein by dry weight, at least 55% protein by dry weight, at least 60% protein by dry weight or at least 75% protein by dry weight. In some embodiments, the algal biomass contains 30-75% or 40-60% protein by dry weight. In some embodiments, at least 40% of the crude protein is digestible, at least 50% of the crude protein is digestible, at least 60% of the crude protein is digestible, at least 70% of the crude protein is digestible, at least 80% of the crude protein is digestible, or at least 90% of the crude protein is digestible. In some embodiments, the microalgal biomass is grown under heterotrophic conditions. In some embodiments, the microalgal biomass is grown under nitrogen-replete conditions.

In embodiments described herein, the microalgal biomass comprises predominantly intact cells. In some embodiments, the food composition comprises oil which is predominantly or completely encapsulated inside cells of the microalgal biomass. In some embodiments, the food composition comprises predominantly intact microalgal cells. In some embodiments, the microalgal oil is predominantly encapsulated in cells of the biomass. In some embodiments, the microalgal biomass comprises predominantly lysed cells (e.g., a homogenate). As discussed above, such a homogenate can be provided as a slurry, flake, powder, or flour.

In embodiments described herein, the food composition comprises the microalgal biomass combined with one or more other edible ingredients, including without limitation, grain, fruit, vegetable, protein, lipid, herb and/or spice ingredients. In some embodiments, the food composition is a salad dressing, egg product, baked good, bread, bar, pasta, sauce, soup drink, beverage, frozen dessert, butter or spread. In some embodiments, the food composition is not a pill or powder. In some embodiments, the food composition weighs at least 50 g, or at least 100 g.

Microalgal biomass can be combined with one or more other edible ingredients to make a food product. The microalgal biomass can be from a single algal source (e.g., strain) or microalgal biomass from multiple sources (e.g., different strains). The biomass can also be from a single algal species, but with different composition profile. For example, a manufacturer can blend microalgae that is high in oil content with microalgae that is high in protein content to the exact oil and protein content that is desired in the finished food product. The combination can be performed by a food manufacturer to make a finished product for retail sale or food service use. Alternatively, a manufacturer can sell microalgal biomass as a product, and a consumer can incorporate the microalgal biomass into a food product, for example, by modification of a conventional recipe. In either case, the algal biomass is typically used to replace all or part of the protein, oil, fat, eggs, or the like used in many conventional food products.

In some embodiments, the food composition formed by the combination of microalgal biomass and/or product derived therefrom, i.e. microalgal flour, protein concentrate, or protein isolate, comprises at least 0.1%, at least 0.5%, at least 1%, at least 5%, at least 10%, at least 25%, or at least 50% w/w or v/v microalgal biomass. In some embodiments, food compositions formed comprise at least 2%, at least 5%, at least 10%, at least 25%, at least 50%, at least 75%, at least 90%, or at least 95% w/w microalgal biomass or product derived therefrom. In some cases, the food composition comprises 5-50%, 10-40%, or 15-35% algal biomass or product derived therefrom by weight or by volume.

Microalgal biomass, including microalgal flour, protein concentrate, or protein isolate, can be incorporated into virtually any food composition. Some examples include baked goods, such as cakes, brownies, yellow cake, bread including brioche, cookies including sugar cookies, biscuits, and pies. Other examples include products often provided in dried form, such as pastas or powdered dressing, dried creamers, comminuted meats, and meat substitutes. Incorporation of predominantly intact microalgal biomass into such products as a binding and/or bulking agent can improve hydration and increase yield due to the water binding capacity of predominantly intact biomass. Re-hydrated foods, such as scrambled eggs made from dried powdered eggs, may also have improved texture and nutritional profile. Other examples include liquid food products, such as sauces, soups, dressings (ready to eat), creamers, milk drinks, juice drinks, smoothies, creamers. Other liquid food products include nutritional beverages that serve as a meal replacement or algal milk. Other food products include butters or cheeses and the like including shortening, margarine/spreads, nut butters, and cheese products, such as nacho sauce. Other food products include energy bars, chocolate confections-lecithin replacement, meal replacement bars, granola bar-type products. Another type of food product is batters and coatings. By providing a layer of oil surrounding a food, predominantly intact biomass or a homogenate repel additional oil from a cooking medium from penetrating a food. Thus, the food can retain the benefits of high monounsaturated oil content of coating without picking up less desirable oils (e.g., trans fats, saturated fats, and by products from the cooking oil). The coating of biomass can also provide a desirable (e.g., crunchy) texture to the food and a cleaner flavor due to less absorption of cooking oil and its byproducts.

In certain embodiments, a protein food bar comprises Euglena Flour at about 5%, 10%, 15%, 20%, 25%, 30%, 35%, 40%, 45%, 50%, 55%, 60%, 65%, 70%, 75%, 80%, 85%, 90%, 95%, until the desired texture was achieved.

In certain embodiments, a protein food bar comprises Euglena beta-glucan isolate at about 5%, 10%, 15%, 20%, 25%, 30%, 35%, until the desired texture was achieved.

In certain embodiments, a protein bar or food product comprises: dates at about 20-35%, agave at about 10-15%, natural peanut butter at about 5-15%, coconut oil at about 1-5%, rolled oats comprising about 5-15%, almonds at about 5-15%, and Euglena flour at about 5-40%, and beta-glucan isolate at about 0-10%.

In certain embodiments, a protein bar or food product comprises: almonds, dates, agave, natural peanut butter, coconut oil, rolled oats, euglena flour, maple syrup, beta-glucan isolate (BGI), Ready to gel (RTG), beta-glucan powder, RTG-wet gel, Tapioca syrup, Agave Syrup, Golden strap Molasses, flavour masking, syrup, and combinations thereof

In certain embodiments, the materials required in the forming of a protein bar food product include a kitchen scale, spatula, spoons, a food processor, kitchen range, double boiler, mixing bowls, wax paper, bar mould, and combinations thereof.

In certain embodiments, a kitchen range is heated to about 330-380° F. The items are combined in such a way that they create a pliable dough. Once dough is formed, used the appropriate cooking apparatus to press a section of the dough into the mould at the desired level. Once the moulds are filled, wrap product in a food preserving material and place in a refrigerator to set.

In uncooked foods, most algal cells in the microalgal biomass remain intact. This has the advantage of protecting the algal oil from oxidation, which confers a long shelf-life and minimizes adverse interaction with other ingredients. Depending on the nature of the food products, the protection conferred by the cells may reduce or avoid the need for refrigeration, vacuum packaging or the like. Retaining cells intact also prevents direct contact between the oil and the mouth of a consumer, which reduces the oily or fatty sensation that may be undesirable. In food products in which oil is used more as nutritional supplement, such can be an advantage in improving the organoleptic properties of the product. Thus, predominantly intact microalgal biomass is suitable for use in such products. However, in uncooked products, such as a salad dressing, in which oil imparts a desired mouth feeling (e.g., as an emulsion with an aqueous solution such as vinegar), use of purified algal oil or micronized biomass is preferred. In cooked foods, some algal cells of original intact microalgal biomass may be lysed but other algal cells may remain intact. The ratio of lysed to intact cells depends on the temperature and duration of the cooking process. In cooked foods in which dispersion of oil in a uniform way with other ingredients is desired for taste, texture and/or appearance (e.g., baked goods), use of micronized biomass or purified algal oil is preferred. In cooked foods, in which microalgal biomass is used to supply oil and/or protein and other nutrients, primarily for their nutritional or caloric value rather than texture.

Microalgal biomass including microalgal flour, protein concentrate, or protein isolate, can also be manufactured into nutritional or dietary supplements. For example, microalgal flour, protein concentrate, or protein isolate can be encapsulated into digestible capsules in a manner similar to other nutritional supplements. Such capsules can be packaged in a bottle and taken on a daily basis (e.g., 1-4 capsules or tablets per day). A capsule can contain a unit dose of microalgal flour, protein concentrate, or protein isolate. Likewise, microalgal biomass can be optionally compressed with pharmaceutical or other excipients into tablets. The tablets can be packaged, for example, in a bottle or blister pack, and taken daily at a dose of, e.g., 1-4 tablets per day. In some cases, the tablet or other dosage formulation comprises a unit dose of microalgal flour, protein concentrate, or protein isolate. Manufacturing of capsule and tablet products and other supplements is preferably performed under GMP conditions appropriate for nutritional supplements as codified at 21 C.F.R. 111, or comparable regulations established by foreign jurisdictions. The microalgal flour, protein concentrate, or protein isolate can be mixed with other powders and be presented in sachets as a ready-to-mix material (e.g., with water, juice, milk or other liquids). The algal biomass can also be mixed into products such as yogurts.

Microalgal biomass, including microalgal flour, protein concentrate, or protein isolate can also be packaged in a form combined with other dry ingredients (e.g., sugar, flour, dry fruits, flavorings) and portioned packed to ensure uniformity in the final product. The mixture can then be converted into a food product by a consumer or food service company simply by adding a liquid, such as water or milk, and optionally mixing, and/or cooking without adding oils or fats. In some embodiments, the liquid is added to reconstitute a dried microalgal biomass composition. Cooking can optionally be performed using a microwave oven, convection oven, conventional oven, or on a cooktop. Such mixtures can be used for making cakes, breads, pancakes, waffles, drinks, sauces and the like. Such mixtures have advantages of convenience for the consumer as well as long shelf life without refrigeration. Such mixtures are typically packaged in a sealed container bearing instructions for adding liquid to convert the mixture into a food product.

### EXAMPLES

### Example 1

### Regulating Protein Content

Protein content is controlled principally by the C:N ratio. Lower C:N ratios yield higher protein content. Regulation of lipid content focuses around the dissolved oxygen content of the reactor. Under hypoxic/anaerobic conditions Euglena converts paramylon to wax esters. Table 1 provides the amount of protein or Beta-glucan produced under different C:N ratios in the culture media.

TABLE 1
C:N ratios for protein or Beta-glucan focused biomass

| | | |
|---|---|---|
| | % Range of | % Range of |
| Carbon:Nitrogen Ratio | Protein in | Beta-Glucan |
| in Media | Biomass | in biomass |

| | | | |
|---|---|---|---|
| 6.23:1 | (Complex) | 25.11 | 54.87 |
| 6.23:1 | (Synthetic) | 28.78 | 51.83 |
| 7.34:1 | (Synthetic), Batch | 49.3 | 36 |
| 7.34:1 | (Synthetic), Batch | 32 | 59 |
| 7.34:1 | (Synthetic) | 32.32 | 48.53 |
| 9.56:1 | (Synthetic) | 40.9 | 43 |
| 9.56:1 | (Synthetic) | 45.4 | 38.7 |
| 9.56:1 | (Synthetic) | 41.44 | 40.91 |
| 9.56:1 | (Complex) | 39.3 | 43.6 |
| 9.56:1 | (Complex) | 38.6 | 48.2 |
| 9.56:1 | (Complex) | 37 | 50.62 |
| 12:1 | (Complex) | 28.7 | 52.9 |
| 12:1 | (Complex) | 38.4 | 43.9 |

20:1

| | | | |
|---|---|---|---|
| 40:1 | (Complex) | 22.8 | 47.4 |
| 80:1 | (Complex) | 16.7 | 59.7 |

### Example 2

### Nutritional Content of Euglena

Based upon a study of publicly available reports, the nutritional contents, up to 59 of the nutritional elements reported of Euglena are provided herein. The complexity of nutrition in Euglena can be considered from a view of “wholesome” or “total nutritional care”, and the flour instead of individual components provides this overall advantage. Nutritional value of Euglena flour depends on environmental conditions.

Proximate and other nutrients are shown in Table 2 below. Cholesterol level was below the detection limit. In terms of GABA, Euglena protein flour contains 417 mg/100 g of GABA. This is in dosage range for some anxiety medications, meaning the protein enriched Euglena dried biomass has additional health properties than just the basic macronutrients.

TABLE 2
Nutrients profile from 100 g Euglena protein
flour. ND represents not detected.

| Nutrient Name | Value | Unit |
|---|---|---|
| Energy (Cal) | 380 | kcal/100 g |
| Calories from Fat | 52 | kcal/100 g |
| Protein | 33 | % |
| Moisture | 5.3 | % |
| Fat | 6.1 | % |
| Ash | 7.4 | % |
| Carbohydrates | 48.2 | % |
| Cholesterol | <2.0 | mg/100 g |
| Total Dietary Fiber | 45.5 | % |
| Total Sugars | 2.5 | % |
| Dextrose | 2.5 | % |
| Fructose | <0.1 | % |
| Lactose | <0.1 | % |
| Maltose | <0.1 | % |
| Sucrose | <0.1 | % |
| chlorophyll | 6.7 | ppm |
| lutein | ND | |
| zeaxanthin | ND | |
| GABA (gamma | 0.417 | % |
| aminobutyric acid) | | |

The fatty acid profile for the 6.1% of fat measured in the 100g Euglena protein flour sample is seen below in Table 3. Of note, trans fatty acids were below the detection limit. As well, approximately half the fatty acids were saturated fatty acids. There was also approximately 20% polyunsaturated fatty acids, such as omega 3, 5 and 9 present in the biomass. This offers the flour some nutritional properties without being a high fat product.

TABLE 3
Fatty acid profile of the Euglena protein flour.

| | | | mg/100 g |
|---|---|---|---|
| | % of FA | % of FA in | of FA in |
| Fatty Acid Name | in flour | total lipids | total flour |
| Lauric Acid (C:12) | 0.1 | 1.72 | 100 |
| Tridecanoic Acid (C13:0) | 0.2 | 3.45 | 200 |
| Myristic Acid (C14:0) | 0.9 | 15.52 | 900 |
| Pentadecanoic Acid (C15:0) | 0.1 | 1.72 | 100 |
| Palmitic Acid (C16:0) | 0.8 | 13.79 | 800 |
| Palmitoleic Acid (C16:1) | 0.2 | 3.45 | 200 |
| Oleic Acid (C18:1n-9) | 0.5 | 8.62 | 500 |
| Vaccenic Acid (C18:1n-11) | 0.3 | 5.17 | 300 |
| Linoleic Acid (C18:2n-6) | 0.1 | 1.72 | 100 |
| alpha-Linolenic Acid (C18:3n-3) | 0.1 | 1.72 | 100 |
| Eicosadienoic Acid (C20:2) | 0.2 | 3.45 | 200 |
| Eicosatrienoic Acid (C20:3n-6) | 0.2 | 3.45 | 200 |
| Tricosanoic Acid (C23:0) | 1.3 | 22.41 | 1300 |
| Eicosapentaeonic Acid (C20:5n-3) | 0.2 | 3.45 | 200 |
| Docosapentaenoic Acid (C22:5n-6) | 0.1 | 1.72 | 100 |
| Docosatetraenoic Acid (C22:14) | 0.4 | 6.9 | 400 |
| Other | 0.1 | 1.72 | 100 |
| Trans Fatty acids | <0.1 | | |
| Total Saturates | 3.3 | 60 | 3300 |
| Total Monounsaturates | 0.9 | 16.36 | 900 |
| Total Polyunsaturates | 1.3 | 23.64 | 1300 |
| Omega-3 Fatty Acids | 0.3 | 5.17 | 300 |
| Omega-6 Fatty Acids | 0.6 | 10.34 | 600 |
| Omega-9 Fatty Acids | 0.7 | 12.07 | 700 |
| Total Fat | 5.8 | | 5800 |

Fatty acids are reported in the percentage of Fatty Acids (FA) in the flour, the percentage of FA in the total lipids as well as a mass in mg per 100 g of the total flour.

The amino acid profile of a protein flour is shown below in Table 4. In total, 27.4 g of protein are in a 100 g serving of Euglena protein flour.

TABLE 4
Amino acid profile in 100 g of Euglena protein flour.

| Amino Acid | g/100 g of biomass | % protein |
|---|---|---|
| Aspartic acid | 2.4 | 8.8 |
| Glutamic acid | 3.7 | 13.4 |
| Serine | 1.1 | 4.0 |
| Histidine | 0.6 | 2.3 |
| Glycine | 1.4 | 5.1 |
| Threonine | 1.4 | 5.0 |
| Arginine | 2.0 | 7.3 |
| Alanine | 2.0 | 7.4 |
| Tyrosine | 0.7 | 2.6 |
| Cystine | 0.1 | 0.5 |
| Valine | 1.9 | 7.1 |
| Methionine | 0.5 | 1.9 |
| Tryptophane | 0.4 | 1.3 |
| Phenylalanine | 1.3 | 4.6 |
| Isolcucine | 1.2 | 4.4 |
| Leucine | 2.4 | 8.9 |
| Lysine | 2.0 | 7.5 |
| Hydroxyproline | 0.8 | 2.8 |
| Proline | 1.4 | 5.0 |
| Total | 27.4 | 100.0 |

Minerals (Table 5) and vitamins (Table 6) both have over 10% the daily value intake in 100g of flour. This offers further nutrition to the products that incorporate the Euglena protein flour.

TABLE 5
Mineral profile in 100 g of Euglena protein flour.

| | | | % Daily |
|---|---|---|---|
| | mg/100 g of | Recommended | value per |
| Minerals | flour | daily Intake (mg) | 100 g flour |
| Calcium | 165 | 1300 | 12.69 |
| Sodium | 1049 | | |
| Iron | 4.1 | 18 | 22.78 |
| Potassium | 99.7 | 4700 | 2.12 |
| Nickel | 0.05 | | |
| Magnesium | 59.9 | 420 | 14.26 |
| Phosphorus | 1435 | 1250 | 114.80 |
| Zinc | 1.6 | 11 | 14.55 |
| Copper | 0.2 | 0.9 | 22.22 |
| Selenium | <0.01 | 0.055 | |
| Manganese | 0.44 | 2.3 | 19.13 |
| Cobalt | 0.01 | | |

TABLE 6
Vitamin profile in 100 g of Euglena protein flour.

| | | | | | % Daily |
|---|---|---|---|---|---|
| | | | Recommended | | Value per |
| Vitamins | Concentration | | daily Intake | | 100 g flour |
| Vitamin A | 0.023 | mg/g | 900 | mcg | 255.56 |
| Vitamin B1 (Thiamin) | 0.0211 | mg/g | 1.2 | mg | 175.83 |
| Vitamin B2 | 0.0211 | mg/g | 1.3 | mg | 162.31 |
| (Riboflavin) | | | | | |
| Vitamin B3 (Niacin) | 0.0285 | mg/g | 16 | mg | 17.81 |
| Vitamin B5 | 0.0606 | mg/g | 5 | mg | 121.2 |
| (Pantothenic acid) | | | | | |
| Vitamin B6 | 0.037 | mg/g | 1.7 | mg | 218.24 |
| Vitamin B7 (Biotin) | <0.1 | ppm | 30 | mcg | N/A |
| Vitamin B9 (Folic acid) | <0.1 | ppm | 400 | mcg | N/A |
| Vitamin B12 | <0.1 | ppm | 2.4 | mcg | N/A |
| Vitamin D | <0.2 | ug/g | 20 | mcg | N/A |
| Vitamin E | 0.25 | mg/g | 15 | mg | 166.67 |

| | | | | |
|---|---|---|---|---|
| Vitamin K1 | 0.0009% | 120 | mcg | 75 |

N/A represents not applicable

### Example 3

### Methods and Examples of Euglena Protein Concentrate

Case 1: Two Phase Protein Production (HS Protein Concentrate and LS Protein Concentrate)

Frozen biomass from harvest was thawed and blended in a commercial blender for 10 minutes. The biomass was diluted with water to approximately 5% solids. The diluted biomass was then neutralized with 1.5 molar sodium hydroxide and then homogenized with a single pass at 12,000 psi in a SPX APV 1000 Lab Series Homogenizer. The homogenate was centrifuged (4700 rpm for 13 minutes) yielding a two phase system, with a light phase of dilute high solubility(HS) protein, referred to as the HS skim phase, and a heavy phase sludge of low solubility (LS) protein mixed with paramylon (PM), referred to as the LS/PM sludge.

The HS skim was collected by decanting, the skim was then adjusted to pH 4.5 with 50% w/w citric acid in water, followed by 1 hour of gentle intermittent stirring before centrifuging at 4,700 rpm for 13 minutes. Two phases were obtained from the HS skim precipitation, a light phase referred to as HS whey (soluble cellular components which did not precipitate under acid) and HS sludge. The HS Sludge was then diluted with an equal mass of water, neutralized with 1.5 M NaOH and then freeze dried, yielding a powdered HS protein concentrate. The HS Whey was discarded. Following freeze drying the HS protein concentrate powder was analyzed by proximate analysis at SGS Mississauga.

To obtain the LS Protein concentrate powder the LS/PM sludge was diluted with an equal amount of water and adjusted to pH 10 with 1.5 M NaOH, while blending in a commercial blender for approximately 10 minutes. Following this alkaline solubilization step, the mixture was centrifuged for 13 minutes at 4,700 rpm yielding two phases: the light phase consisted of the solubilized low solubility protein (LS Skim) and a heavy phase of paramylon sludge. The LS Skim was decanted off prior to precipitation of the proteins by adjusting the pH to 4.5 with 50% w/w aqueous citric acid. After 1 hour at pH 4.5 with gentle agitation, the LS Skim was centrifuged for 13 minutes at 4,700 rpm, yielding a light phase of acid soluble cellular material (LS whey) and a precipitate of the low solubility proteins, called LS sludge. The LS sludge was diluted with an equal mass of water before neutralizing with 1.5 M NaOH, and freeze drying, yielding the LS protein concentrate powder, which was also subject to proximate analysis as seen in Table 7.

TABLE 7
Proximate Data for Case 1 Two Phase Protein Production

| | % Protein | % Fat | % Carbohydrates | % Ash |
|---|---|---|---|---|
| Sample | (dry basis) | (dry basis) | (dry basis) | (dry basis) |
| Starting Biomass | 34.7 | 4.4 | 50.6 | 10.3 |
| HS Protein | 68.5 | 21.0 | 1.6 | 8.9 |
| Concentrate | | | | |
| Powder | | | | |
| LS Protein | 68.6 | 12.2 | 11.9 | 7.5 |
| Concentrate | | | | |
| Powder | | | | |

Case 2: Post-Homogenization Alkaline Separation

Frozen biomass from harvest was thawed and blended in a commercial blender for 10 minutes. The biomass was diluted with water to approximately 5% solids with water. The diluted biomass was then neutralized with 1.5 molar sodium hydroxide and then homogenized with a single pass at 12,000 psi in a SPX APV 1000 Lab Series Homogenizer. The homogenate was immediately adjusted to pH 10 with 1.5 M NaOH. The pH adjusted homogenate was stirred gently for 1 hour to ensure maximal protein solubilization obtainable under these conditions. After stirring, the homogenate was centrifuged for 13 minutes at 4,700 rpm.

Following centrifugation, only two phases were obtained: a heavy phase consisting mostly of paramylon (PM sludge) and a light phase containing dissolved proteins and any other mildly alkaline soluble cellular components (PC Skim). The PC skim phase was then adjusted to pH 4.5 using 50% w/w aqueous citric acid. Following pH adjustment, the mixture was stirred gently overnight (˜16 hours) prior to centrifugation at 4,700 rpm for 13 minutes. This separation yielded a light phase consisting of alkaline and acid soluble cell components (PC Whey) and a heavy sludge phase containing the precipitated proteins (PC sludge). The PC sludge was then diluted with an equal mass of water, neutralized with 1.5 M NaOH and then spray dried using the LabPlant SD-06 spray dryer, with an inlet temperature of 160 C at a feed rate of approximately 10 mL/min. The powdered protein concentrate obtained was sent for proximate analysis at SGS-Mississauga as seen in Table 8.

TABLE 8
Proximate Data from Case 2 Post-Homogenization
Alkaline Separation

| | % Protein | % Fat | % Carbohydrates | % Ash |
|---|---|---|---|---|
| Sample | (dry basis) | (dry basis) | (dry basis) | (dry basis) |
| Starting Biomass | 34.7 | 4.4 | 50.6 | 10.3 |
| Protein | 74.4 | 17.2 | 0.1 | 8.3 |
| Concentrate | | | | |
| Powder | | | | |

Case 3: Pre-Homogenization Alkaline Extraction

TK4300 biomass from two harvests were thawed from frozen, combined, and diluted to 5% solids with water. The diluted biomass blend was then mixed in an agitated kettle for 15 minutes. Following resuspension and mixing, the biomass was adjusted to pH 10 using 1.5 M NaOH. Following pH adjustment the biomass was then homogenized at 12,000 psi in a SPX APV 1000 Lab Series Homogenizer. The homogenate obtained was then centrifuged for 13 minutes at 4,700 rpm, yielding two phases, a light phase containing solubilized protein and other alkaline soluble cell components (PC Skim) and a heavy phase consisting mainly of paramylon (PM sludge). The PC skim was adjusted to pH 4.5 using 85% w/w phosphoric acid, followed by gentle agitation for 1 hour to complete the precipitation. After the precipitation reaction, the PC skim was centrifuged at 4,700 rpm for 13 minutes yielding two phases, a light phase consisting of acid and alkaline soluble components (whey) and a heavy phase consisting of the precipitated proteins (PC sludge).

The PC sludge was diluted to 10% solids, adjusted to pH 7 using 1.5 M NaOH and spray dried using a 35″ Wild Horse International Spray Dryer, S/S, Model LPG-5 with an inlet temperature of 250 C and variable flow rate to maintain an outlet temperature of 70-75 C. The protein concentrate powder obtained was sent for proximate analysis at SGS Mississauga as seen in Table 9.

TABLE 9
Proximate Data from Case 3 Pre-Homogenization
Alkaline Extraction

| | % Protein | % Fat | % Carbohydrates | % Ash |
|---|---|---|---|---|
| Sample | (dry basis) | (dry basis) | (dry basis) | (dry basis) |
| Starting Biomass | 43.4 | 12.6 | 37.6 | 6.7 |
| Protein | 70.4 | 21.9 | 0.5 | 7.2 |
| Concentrate | | | | |
| Powder | | | | |

Case 4. Acidic Homogenization Process

TK4300 biomass from two harvests were thawed from frozen, combined, and diluted to 5% solids with water. The diluted biomass blend was then mixed in an agitated kettle for 15 minutes. The diluted biomass was adjusted to pH 4.7 using 1.5 M NaOH (starting pH 3.3). The biomass was then homogenized at 12,000 psi in a SPX APV 1000 Lab Series Homogenizer. The homogenate was stored overnight in a fridge at 40 C. The next day the homogenate was centrifuged for 13 minutes at 4,700 rpm, yielding a three phase mixture; the lightest phase contained acid soluble cellular components (Whey), the sludge obtained formed two somewhat discrete phases with a horizon separating the upper part, composed mainly of precipitated proteins (PC sludge), and the lower part, consisting mainly of the denser paramylon (PM sludge). The whey was decanted, and discarded. The PC sludge was then manually removed from the PM sludge by use of hand tools in the centrifuge bottles and collected. The PM sludge was separately processed into beta-glucan isolate.

The PC sludge was then diluted to 10% solids, adjusted to pH 7 and spray dried using a 35″ Wild Horse International Spray Dryer, S/S, Model LPG-5 with an inlet temperature of 250 C and variable flow rate to maintain an outlet temperature of 70-75 C. The protein concentrate powder was collected and sent for proximate analysis at SGS Mississauga as seen in Table 10.

TABLE 10
Proximate Data from Example 4 Acidic Homogenization Process

| | % Protein | % Fat | % Carbohydrates | % Ash |
|---|---|---|---|---|
| Sample | (dry basis) | (dry basis) | (dry basis) | (dry basis) |
| Starting Biomass | 43.4 | 12.6 | 37.6 | 6.7 |
| Protein | 60.3 | 15.8 | 16.0 | 7.8 |
| Concentrate | | | | |
| Powder | | | | |

Case 5: Examples Of Future Optimizations Within Process for Protein Concentrate and Isolate

The examples as laid out above rely on multiple key unit operations which can be further optimized. The operations which warrant further optimization include, but are not limited to: homogenization, precipitation, phase separation, and drying.

Homogenization (and alternate cell lysis technology): The homogenization unit operation has multiple parameters which have yet to be completely optimized including, but not limited to: solid content of biomass, temperature of homogenization, number of passes, homogenization pressure and homogenization valve geometry and number of stages. For example modifying the solids content of homogenization can influence both the lysis effectiveness as well as whether certain biomolecules associate (ie proteins and lipids) in concentration dependent manner. Optimizing temperature will further influence the thermodynamics of biomolecule separation, as well as potentially limiting the thermal denaturation of proteins, which will lead to more highly functional concentrates and isolates. The pass number of the homogenizer as pressure and geometry will all directly influence the separation of protein from lipids, as over homogenization leads to emulsification which negatively impacts protein and lipid separation dramatically. By minimizing the lipid emulsification through gentler lysis, it is expected a higher protein content concentrate and/or isolate will be obtained. Furthermore, alternative cell lysis technologies may also be utilized to achieve the same effect, including, but not limited to: enzymatic lysis, ultrasonic lysis, chemical lysis or milling.

Precipitation: The precipitation operation may be influenced by the choice of acid, or other precipitant used, the concentration of the acids and bases used for adjustment, the agitation mechanism and speed, the temperature and the time used for the reaction. Different acids by virtue of their different chemical structures interact differently with proteins and can modify their structure and thus solubility and functionality; further work will examine different acids as well as other non-acid precipitants such as ammonium sulfate or other food safe salts on the Hofmeister Series that can be used to salt-out proteins from solution. Agitation mechanisms and speeds can have significant impact on protein solubility by virtue of whether they introduce air or not. Introduction of air generates air-liquid interfaces at which proteins must modify their structure which can ultimately change their final functional properties upon recovery. An optimal agitation mechanism will be selected so as to not perturb the native protein structure and maintain optimal functionality. The time of the reaction will dictate the yield and the amount of contaminants which are copurified during the subsequent unit operations. Alternatively, precipitation may be side-stepped by selective removal of other contaminants, like lipids and carbohydrates through use of chromatographic technologies which selectively adsorb either the proteins or impurities, prior to the drying process.

Isoelectric Precipitation:

The clarified product may be transferred to a tank equipped with pH, temperature and agitation controlling sensors. The pH may be adjusted to expedite the isoelectric precipitation under controlled conditions (e.g., protein concentration, agitation time, temperature and shear rate). The isoelectric precipitation can be performed at different stages to precipitate the maximum soluble protein from the centrate. Further, the heat treatment may be applied to accelerate the precipitation process. After that, the precipitate may be recovered by continuous centrifugation (disc stack centrifuge) employing the optimized operating process parameters (g force, feed flow rate back pressure and discharge time) to achieve the desired solid content.

Alternatively, the precipitate can also be separated through a sedimentation process in a sedimentation tank. Further, the precipitation process can be accelerated by using the food grade flocculants.

Phase Separation: The phase separation operation presents many options for future optimization, such as modification of centrifuge technology (disc-stack, decanter, tubular bowl, etc), the parameters of the centrifuges (feed rate, speed, weir depth, etc). Different centrifuges and operating parameters will allow for modification of the applied forces which influence the mechanical separation of the precipitated proteins from the surrounding aqueous environment, which will ultimately lead to higher protein content in the concentrates produced by virtue of better exclusion of contaminants such as lipids and carbohydrates. Alternatively, different phase separation technologies, orthogonal to centrifugation will be tested, including but not limited to, ultrafiltration and chemically induced phase partitioning. Ultrafiltration may be used to concentrate the proteins from the lysate, and may not require the use of precipitants at all, which may allow higher protein contents to be achieved in the concentrates, as well as improved functionality of the final products. Chemical phase partitioning may be performed by addition of water soluble polymers such as polyethylene glycol to the system, which lead to multiphase aqueous mixture to form, wherein there are polymer rich and polymer deficient phases. Proteins will also selectively partition into these phases in a way such that they then may be separated from other cellular components by simple decanting or accelerated mechanical separation of the newly formed phases, with subsequent separation of the proteins from the polymers.

Membrane Filtration:

Membrane filtration is a separation technique, which is extensively used in bioprocessing. Depending on membrane porosity, it can be classified as a microfiltration or ultrafiltration process. Microfiltration membranes are generally used for clarification, sterilization and removal of micro-particulates or for cell separation. Ultrafiltration membranes are generally used for concentrating and desalting dissolved molecules (proteins, peptides, nucleic acids, etc.), buffers exchange and fractionation. The two common ways of membrane process operation are dead-end (Normal Flow Filtration) and cross-flow (Tangential Flow Filtration) filtration modes. In the cross-flow filtration mode, the fluid requiring filtration flows parallel to the membrane surface to retain the high molecular weight molecules and pass the water and low molecular weight solutes in the permeate due to a pressure difference across the membrane. The cross-flow reduces the formation of filter cake to keep it at a low level. The two main variables transmembrane pressure-TMP and cross-flow rate or feed flow rate, are controlled in all tangential flow devices during UF/DF operation.

The ultrafiltration may be used to concentrate the soluble proteins from the centrate. The right molecular weight cutoff (MWCO) membrane used to retain the maximum proteins may be selected based on the screening experiment. Then, the operating process parameters can be optimized to achieve the maximum flux. Further, the number of the diafiltration can be performed to achieve the higher protein content or to make the protein isolate.

Drying: The drying operation presents another opportunity to both optimize the purity of the protein concentrate and/or isolate, as well as the functionality. The purity may be influenced by use of air flotation separation or triboelectric separation concurrent with the dryer, which can separate protein from other components based on density and charge, respectively. The functionality of the final products is significantly influenced by the drying technology and optimization of this operation is imperative for highly functional protein products. Proteins are easily thermally denatured, which leads to a change in their structure in response to heat from their environment; most drying technologies utilize heat and thus present a direct challenge to the protein structure and function. Careful optimization of drying technology allows the minimal amount of heat to be applied to the product, for example by using the lowest possible inlet temperature in a spray dryer which yields a sufficiently low moisture product. Alternative drying methods may be selected and optimized such as freeze drying or vacuum oven drying which potentially introduce even less heat to the proteins, better maintaining their native structure and ultimately, their functionality in food systems.

The protein concentrate or protein isolate slurry generated through the precipitation or by membrane filtration methods can be spray dried to obtain the dried powder of protein concentrate or protein isolate. The spray drying can be performed by passing the slurry through a spray dryer at the optimized key operating process parameters (Total solids, Feed flow rate, Atomizing air pressure, Inlet temperature and Outlet temperature) to achieve the desired moisture content of the powder.

### Example 4

### Defatting of Protein Concentrate and Flour and its use for Protein Isolate

Introduction: The purpose of this example is to determine the most promising food acceptable solvent in the defatting of protein flour and concentrate. Three common food grade organic solvents are tested: Ethanol, isopropanol and hexane. The final products are measured for their protein and lipid content to determine the successfulness of defatting, and the resultant final protein concentration.

Methods: 4 grams of sample Protein Concentrate (PC) and Protein Rich Euglena Flour (PF) was mixed in 40 mL of solvent (hexane, isopropanol, ethanol) for 24 hours at room temperature on a stir plate. The samples were then centrifuged (3500 rpm, 5 minutes). The supernatant was decanted into a new 50 mL tube. The pellet was resuspended in 40 mL of respective solvent and vortexed for 20 seconds to mix. The sample was centrifuged again (3500 rpm, 5 minutes). The supernatant was kept in a separate 50 mL tube. The pellet and supernatant tubes were evaporated using the GeneVac EZ-2 evaporator. The samples of the starting materials and defatted pellets were sent for total nitrogen analysis at the Water Quality Centre at Trent University. Total lipid extraction was performed on the defatted samples as well as the starting samples using an internal Lipid Extraction for GC Analysis method that relies on chloroform and methanol to form a monophasic solvent system to extract and dissolve the lipids.

Results/Discussion: The first major result was the observation that the crude fat analysis as performed by SGS yielded significantly lower values (10-14% lower) than the total lipid extraction method utilized internally. The large discrepancy between these two extractions suggests a large fraction of Euglena lipid, in these samples, was not soluble in ether (crude fat), but soluble in the 2 chloroform: 2 methanol: 1.8 water extraction mixture used for total lipid analysis. The most likely reason in this instance is a high degree of polar lipids such as phospholipids being present which would have only limited solubility in the relatively nonpolar ether.

Protein concentrate was found to be more readily defatted, going from 48.4% total lipid to approximately 20% after defatting with any of the solvents tested, than flour, which went from 32.2% to approximately 20% lipid content after defatting with any of the solvents tested. These results show that concentrate had 75% of its lipids removed whereas flour only had about 50% of its lipids removed. These results are logical in the sense that protein concentrate is homogenized, and thus solvents would have an easier time dissolving out the lipid component, compared to the semi-intact cells present in flour. These results are supported in both Tables 11 and 12.

TABLE 11
Lipid content before and after defatting

| | Before (%) | | Total Lipid After (%) | | |
|---|---|---|---|---|---|
| Sample | Crude Fat | Total Lipid | Ethanol | Isopropanol | Hexane |
| Protein | 34.2 | 48.4 | 18.07 | 19.11 | 19.69 |
| Concentrate | | | | | |
| Protein Rich | 22.2 | 32.2 | 17.75 | 17.72 | 20.26 |
| Flour | | | | | |

TABLE 12
Lipid Mass Balance

| | | Starting | Starting | Defatted | Lipid in | Lipid |
|---|---|---|---|---|---|---|
| | | sample | lipid | residue | residue | removed |
| Sample | Solvent | (g) | (g) | (dry)(g) | (g) | (%) |
| Protein | Ethanol | 4.002 | 1.937 | 2.541 | 0.459 | 76.3 |
| Concen- | Isopro- | 4.005 | 1.938 | 2.538 | 0.485 | 75.0 |
| trate | panol | | | | | |
| | Hexane | 4.000 | 1.936 | 2.528 | 0.498 | 74.3 |
| Protein | Ethanol | 4.001 | 1.288 | 2.702 | 0.480 | 63.0 |
| Rich | Isopro- | 4.007 | 1.290 | 2.805 | 0.497 | 61.5 |
| Flour | panol | | | | | |
| | Hexane | 4.005 | 1.290 | 2.883 | 0.584 | 54.7 |

The results of Table 13 and 14 indicate that in this defatting experiment, along with lipid, a large amount of protein was extracted into the various organic solvents tested. Because of this the lipid extracts themselves are unlikely to be purely lipid, potentially due to a high amount of lipoprotein complexes being present in the sample. Further work should investigate the protein content of the evaporated lipid concentrates. Protein solubility was found to be highest in ethanol in both protein concentrate and flour. For example, 87.4% of the N content (the proxy for protein in this experiment) was lost to the solvent from protein rich flour with ethanol extraction. It is unexpected that the proteins have such high solubility in these solvents, and perhaps the centrifugation was insufficient for recapture of the insoluble protein components. Alternatively, the lipid of Euglena may have an abnormally high N content (eg. phosphatidylcholine), causing this bias in the mass balance. However, this must be explored further, as it could reveal a very interesting functionality of the Euglena protein being highly soluble in organic solvents. An option to recapture suspended insoluble protein could be to utilize centrifugation and filtration, rather than centrifugation alone, such as gravity or vacuum filtration. Future work could also investigate the use of higher temperature during extraction which may disrupt interaction between lipid and protein, thus preventing protein carryover into the lipid extract.

TABLE 13
Protein content before and after defatting

| | | After % | | |
|---|---|---|---|---|
| Sample | Before (%) | Ethanol | Isopropanol | Hexane |
| Protein | 49.3 | 49.56 | 64.19 | 60.25 |
| Concentrate | | | | |
| Protein Rich | 34.2 | 12.63 | 45.69 | 33.50 |
| Flour | | | | |

TABLE 14
Protein Mass Balance

| | | | | | Protein | Protein |
|---|---|---|---|---|---|---|
| | | Starting | Starting | Defatted | in | lost to |
| | | sample | protein | residue | residue | solvent |
| Sample | Solvent | (g) | (g) | (dry)(g) | (g) | (%) |
| Protein | Ethanol | 4.002 | 1.973 | 2.541 | 1.259 | 36.2 |
| Concen- | Isopro- | 4.005 | 1.974 | 2.538 | 1.629 | 17.5 |
| trate | panol | | | | | |
| | Hexane | 4.000 | 1.972 | 2.528 | 1.523 | 22.8 |
| Protein | Ethanol | 4.001 | 1.368 | 2.702 | 0.341 | 75.1 |
| Rich | Isopro- | 4.007 | 1.370 | 2.805 | 1.282 | 8.8 |
| Flour | panol | | | | | |
| | Hexane | 4.005 | 1.370 | 2.883 | 0.966 | 29.5 |

From the work in Example 3, it was observed that there was a 75% decrease in lipids with isopropanol extraction and a lost of 17.5% protein. When looking at the starting biomass of 49.3% protein, 48.4 lipids, and 2.3% other (such as ash and carbs), if we apply the same logic, we would assume that there is a final protein percentage of 73.9%, with 21.97% lipids, and 4.13% other. Assuming there is no protein loss in the solvent through modifications, the protein percentage could be as high as 77.4%.

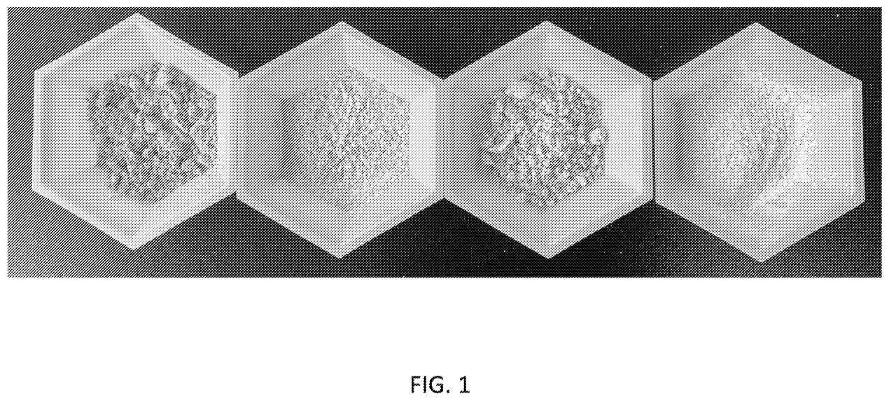

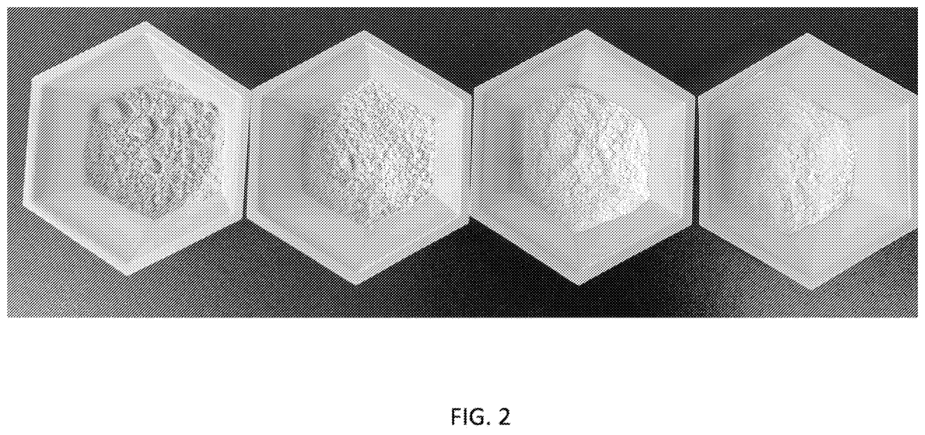

Defatting was found to be useful in control of color in both protein concentrate (FIG. 1) and protein flour (FIG. 2). These results suggest that the colors of the Euglena powder (yellow/orange) are due to lipid soluble components, such as carotenoids. If color is of concern for product applications, solvent defatting may be utilized to increase the neutrality of the protein color attributes.

Other solvents that could be used to remove lipids from protein sample include: Acetone, benzyl alcohol, 1,3-Butylene Glycol, Carbon Dioxide, Castor Oil, Citric Acid Esters of Mono- and Di-glycerides, Ethyl Acetate, Ethyl Alcohol (Ethanol), Ethyl alcohol denatured with methanol, Glycerol (Glycerin), Glyceryl diacetate, Glyceryl triacetate (Triacetin), Glyceryl tributyrate (Tributyrin), Hexane, Isopropyl alcohol (Isopropanol), Methyl Alcohol (methanol), Methyl ethyl ketone (2-Butanone), Methylene Chloride (Dichloro-methane), Monoglycerides and diglycerides, Monoglyceride citrate, 1,2-Propylene glycol (1,2-propanediol), Propylene glycol mono-esters and diesters of fat-forming fatty acids, and Triethyl citrate.

Conclusions: Protein concentrate and flours were defatted using ethanol, isopropanol and hexane. Protein concentrate was more readily defatted than protein flour. Nitrogen was carried over into the lipid extracts, potentially interfering with results, suggesting protein loss, or a high degree of nitrogen content in the Euglena lipid. The color of defatted residues was much more neutral than the starting materials. Next steps include: Investigating the protein content of the lipid extract, comparing crude fat extract (petroleum ether) to the extracts prepared in these experiments, and investigating temperature impact on lipid extraction.

Conclusions Drawn in terms of Euglena Protein Isolate: This work illustrates the ability of removing 75% of fat in a protein concentrate sample. It also illustrates that on average, without any modifications there is 17.5-36.2% of protein (nitrogen) loss in the lipid extraction solvent, which varies based on solvent used. Of the three tested here, isopropanol had the lowest protein loss at 17.5%. Based on this, it can be inferred that if a material that had a higher starting protein percentage, such as those observed in Example 3, with defatting by solvent extraction, an Euglena protein isolate with greater than 80% protein can be obtained. For example, if the proximate data from Example 3 was looked at, shown here again in Table 15:

TABLE 15
Proximate Data from Case 3 Pre-Homogenization
Alkaline Extraction

| | % Protein | % Fat | % Carbohydrates | % Ash |
|---|---|---|---|---|
| Sample | (dry basis) | (dry basis) | (dry basis) | (dry basis) |
| Starting Biomass | 43.4 | 12.6 | 37.6 | 6.7 |
| Protein | 70.4 | 21.9 | 0.5 | 7.2 |
| Concentrate | | | | |
| Powder | | | | |

The protein concentrate powder is at 70.4% protein, with 21.9% lipids, low amount of carbs and some ash. If there are the following assumptions, as shown in the calculations below, and this biomass was defatted by isopropanol solvent extraction, it is expected that on average, the protein isolate would have a 81.5% protein content.

For these calculations, an 100g sample of Euglena protein concentrate powder is used, with the proximate data shown in Table 15.

Using isopropanol, 75% lipid removal is expected which results in: 21.9 g (0.75)=16.4 g lipid removed, 5.5 g lipid remaining. If isopropanol is used, it is expected to have an average 17.5% protein loss. So of the original 70.4 g of protein: 70.4 g(0.175)=we would lose 12.32 g of protein, and have 58.1 g protein remaining. In this example, it is also assumed that there is no loss of carbs or ash in the solvent extraction, meaning that there is still 0.5 g carbs and 7.2 g Ash. Because of this, the new total mass is as follows: 58.1 g Protein+5.5 g lipid+7.2 g Ash+0.5 g carb=71.3 g total mass.

Based on these assumptions, it is assumed that there is 58.1 g of protein in a 71.3 g sample, or the sample contains 81.5% protein. The new lipid content then would also be 7.7%, carb content would be 0.7% and the new ash content would be 10.1%. A summary of this is found in Table 16 below highlighting the change in protein and lipid content. Thus in this example, a protein isolate would have a protein content of 81.5%. In theory, if the solvent method was improved to minimize the loss of protein in the solvent extraction, such as vacuum filtration, the protein content could be as high as 84.2%. If the Euglena protein concentrate from Example 3, Table 8, was defatted using the same logic with isopropanol, it would generate a protein isolate with 82.9% protein content, assuming protein loss in the solvent, or 85.4% if methods were improved to prevent protein loss in solvent.

TABLE 16
Summary table of changes between Protein concentrate
to Protein isolate with an isopropanol solvent extraction.
Numbers are given as percent dry basis

| | % Protein | % Lipids(dry | % Carbs | % Ash |
|---|---|---|---|---|
| Sample | (Dry basis) | basis) | (dry basis) | (dry basis) |
| Protein | 70.4 | 21.9 | 0.5 | 7.2 |
| Concentrate | | | | |
| Lipid removal | loss of protein in | 75% lipid | Assume | Assume |
| by solvent | solvent 0-17.5% | removal | unchanged | unchanged |
| extraction | | | | |
| Protein Isolate | 81.5-84.2% | 7.7 | 0.7% | 10.1% |

### Example 5

### Protein Flour and Protein Concentrate Amino Acid Profile, Digestibility and PDCAAS Score

In this study, the amino acid profile, total protein, and PDCAAS (Protein Digestibility Corrected Amino Acid Score) values were investigated for different samples of Euglena protein flour, as well as protein concentrate. Samples were sent to a third party analytical lab to determine the percentage of amino acids present in the sample, the total protein and PDCAAS number.

The amino acid profiles, PDCAAS (Protein Digestibility Corrected Amino Acid Score) and digestibility are reported in Table 17 for protein concentrate and protein flour. Total protein varied between 33-48% for the protein concentrate samples (n=2) and 18-38% for the protein rich flour (n=4). The total protein was higher on average in the protein concentrate samples compared to the protein rich flour, as expected.

For the protein concentrate, the PDCAAS score was 0.96 and 0.73. A value of 1 represents the complete reference protein, accordingly 0.96 is a very high score and 0.73 is still a good indicator of protein quality. In terms of the protein rich flour, the PDCAAS value varied from 0.93 to 1.21. PDCAAS Scores that are greater than 1 indicate that these samples have higher levels of essential amino acids. This indicates that the proteins in the protein enriched Euglena flour are of high quality and high digestibility as they a near 1 or exceed 1. PDCAAS scores above 1 are rounded down to 1 as the accepted score.

TABLE 17
Protein concentrate and protein flour amino acid profile, digestibility and PDCAAS Score.

| | Protein | Protein | Protein- | Protein- | Protein- | Protein- | Protein- |
|---|---|---|---|---|---|---|---|
| | Concentrate | Concentrate | Rich | Rich | Rich | Rich | Rich |
| Product | A | B | Flour A | Flour B | Flour C | Flour C | Flour D |
| L-Cysteine + | 2.01 | 2 | 0.76 | 1.56 | 1.13 | 1.25 | 1.23 |
| L-Methionine* | | | | | | | |
| L-Tryptophan* | 0.73 | 0.68 | 0.33 | 0.52 | 0.45 | 0.37 | 0.53 |
| L-HydroxyProline | 0 | 0 | 0 | 0 | 0 | 0 | 0 |
| L-Aspartic acid | 5.5 | 3.08 | 1.76 | 3.63 | 2.87 | 2.71 | 3.09 |
| L-Threonine* | 1.64 | 1.62 | 0.92 | 1.66 | 1.49 | 1.4 | 1.64 |
| L-Serine | 2.08 | 1.41 | 0.88 | 1.66 | 1.35 | 1.25 | 1.45 |
| L-Glutamic Acid | 6.07 | 3.96 | 2.43 | 4.57 | 3.72 | 3.66 | 4.11 |
| L-Proline | 2.21 | 1.84 | 0.94 | 2.11 | 1.78 | 1.68 | 1.95 |
| L-Glycine | 2.22 | 1.66 | 0.91 | 1.84 | 1.45 | 1.45 | 1.64 |
| L-Alanine | 3.61 | 2.4 | 1.39 | 2.75 | 2.14 | 2.19 | 2.59 |
| L-Valine* | 2.65 | 2.25 | 1.21 | 2.47 | 1.89 | 1.96 | 2.33 |
| L-Isoleucine* | 2.05 | 1.46 | 0.8 | 1.58 | 1.17 | 1.23 | 1.5 |
| L-Leucine* | 4.37 | 2.85 | 1.67 | 3.06 | 2.39 | 2.36 | 2.86 |
| L-Tyrosine + | 4.34 | 2.89 | 1.41 | 3.21 | 2.34 | 2.22 | 2.77 |
| L-Phenylalanine* | | | | | | | |
| L-Lysine* | 3.97 | 2.4 | 1.29 | 2.49 | 2.26 | 2.15 | 2.56 |
| L-Histidine* | 1.04 | 0.96 | 0.67 | 1.1 | 0.89 | 0.9 | 0.97 |
| L-Arginine | 3.8 | 2.45 | 1.01 | 3.93 | 0.29 | 3.31 | 2.43 |
| Total Protein | 48.29 | 33.91 | 18.38 | 38.14 | 27.61 | 30.09 | 33.65 |
| Moisture % | 4.56 | 2.2 | 2.7 | 5.1 | 6.8 | 9.48 | 4.4 |
| Protein % (Fresh | 52.6 | 51.9 | 21.5 | 42 | 30.8 | 35.6 | 32.3 |
| Weight Basis) | | | | | | | |
| In Vitro | 1.04 | 0.91 | 0.9 | 1.14 | 0.82 | 1.07 | 0.9 |
| Digestibility | | | | | | | |
| First Limiting | L- | L- | L- | L- | L- | L- | L- |
| Amino Acid | Tryptophan | Lysine | Lysine | Lysine | Leucine | Tryptophan | Leucine |
| Amino Acid | 0.917 | 0.798 | 1.034 | 1.022 | 1.175 | 0.954 | 1.343 |
| Score | | | | | | | |
| PDCAAS | 0.96 | 0.73 | 0.93 | 1.17 | 0.96 | 1.03 | 1.21 |

Asterisks represent the 9 essential amino acids. Amino acid values are given as a percentage

### Example 6

### Egg Replacer with Euglena Protein Flour, Beta-Glucan Isolate, and Ready-To-Gel Powder

In the market, there are 2 different egg replacement products strategies one is targeted for bakery applications. In that application, the main ingredients are starches and gums (used as binders and texturizers) accompanied by leavening agents to make up for egg white's leavening functionality in baking applications, along with some protein.

The other application for an egg replacer is a powder or liquid egg replacer that can scramble, for use as scrambled eggs or omelets. For the scramble egg replacer, the main ingredients will include a plant protein (for mimicking the nutrition of the real egg), and a mixture of different gums/hydrocolloids as binder and texture developers upon cooking.

In this study, a combination of pea protein concentrate and Euglena protein flour was used as the main protein source. Beta-glucan ready to gel (RTG) powder was used as the sole hydrocolloid source to act as a binder/texturizer. Beta-glucan RTG powder is solubilized beta-glucan in 1 M NaOH, that has been formed into a gel with 3.75% citric acid, and then freeze dried to form a powder. When the powder is put back into water, it readily forms a thickened solution or gel, depending on the concentration. The addition of beta-glucan isolate in combination with the Euglena protein flour gives the egg scramble the expected yellow colour of a scrambled egg, meaning that a coloring agent is not needed in these applications. In addition, the addition of the beta-glucan isolate also showed a masking effect on the Euglena protein flour off-notes compared to control mixture without the beta-glucan isolate. The Euglena flour is yellow which also adds to the expected yellow colour of scrambled eggs.

No flavoring agent or flavor masker has been used in this formulation and still panelists could perceive an umami-like taste which would be attributed to the protein sources (combination of Euglena flour and pea protein).

Methods and Materials: The formulation that was used in this study for an egg scramble is written in Table 18 which included the Euglena protein flour at approximately 30% protein in the flour, beta-glucan isolate from Euglena, as well as ready to gel beta-glucan powder to act as the hydrocolloid source. The dry ingredients are mixed together, then water is added and mixed. The mixture is whisked for 1 minute. To test the scramble like properties, the whisked mixture is poured into a frying pan that has been set to medium heat (approximately 170° C.) and with 1 tablespoon of warmed oil (i.e. vegetable, 1 tablespoon per 100 grams of liquid egg replacer). The whisked mixture is fried for about 7 to 9 minutes with a scrambling action every minute to two minutes to obtain a scrambled egg consistency. The mixture was analyzed by an internal taste panel.

TABLE 18
Formulation of egg (scramble) replacer

| | % | % |
|---|---|---|
| Egg Replacer Formulation | (Wet basis) | (Dry basis) |
| Water | 87.8 | 0.0 |
| Euglena Protein Flour* | 4.0 | 32.8 |
| Pea Protein Concentrate | 3.0 | 24.6 |
| (80% protein content) | | |
| Beta-glucan isolate powder | 1.0 | 8.2 |
| Ready to gel beta-glucan powder | 4.0 | 32.8 |
| Salt | 0.2 | 1.6 |
| Total | 100.0 | |
| % Total Solid | 12.2 | |

*Euglena protein Flour Spec:
Protein: 35.63%
Oil: 13.4%
Carbs: 38.69%

Results and Discussion: The Euglena based liquid scramble egg replacer had a similar yellow colour to that of control scrambled egg. In this case, no flavouring agent or other known flavour masker was added in this formulation. As such in this formulation, the taste panelist could detect an umami-like taste which is due to the protein sources of the Euglena protein flour and the pea protein. Onion powder or salt, garlic powder or salt, and/or nutritional yeast powder could be a flavouring agent added to improve the taste of the Euglena liquid egg replacer.

### Example 7

### Additional Studies on Liquid Egg Replacer

In this study, the effect of Gellan gum as a hydrocolloid texturizer/binder, onion powder as a flavouring agent, and a higher protein enriched Euglena flour was investigated.

Materials and Methods: Euglena liquid egg replacer is seen in Table 19. For the Euglena protein flour, a flour with 37% protein and 48% protein was investigated to determine the effects of higher protein inclusion flour. In this formulation, no beta-glucan isolate as a masker/whitening agent was used. As well, no RTG beta-glucan powder was used as a hydrocolloid. Instead, the hydrocolloid gellan gum was added. A flavouring agent of onion powder is also added to improve taste of the final product.

To form the mixture, all dry ingredients were mixed, including the gellan gum followed by water addition and mixing. The mixture was heated to 80° C. for 1 hour, up to 2 hours for the gellan gum to hydrate and gel. The mixture is poured into a frying pan that has been set to medium heat (approximately 170° C.) with 1 tablespoon of warmed oil (i.e. vegetable, 1 tablespoon per 100grams of liquid egg replacer). The mixture is fried for about 7 to 9 minutes with a scrambling action every minute to two minutes to obtain a scrambled egg consistency. The mixture was analyzed by an internal taste panel. The experiment was repeated again to confirm results.

TABLE 19
Ingredient list of Euglena liquid egg replacer.

| | % |
|---|---|
| Ingredients | (Wet basis) |
| Water | 85.42 |
| Euglena Protein Flour (37% or 48%) | 8.00 |
| Pea Protein Concentrate (80%) | 6.00 |
| Salt | 0.10 |
| Gellan Gum Powder | 0.18 |
| Onion Powder | 0.30 |
| Total | 100.00 |

Results and discussion: While after 1 hour heat treatment of the lower Euglena protein flour egg prototype (37% protein content), the presence of the gellan gum creates a thickened gel like consistency that resembles liquid egg and helps with the desired texture of scrambled egg upon cooking. In the prototype with higher Euglena protein content (47%) under the same heat treatment condition for 1 hour and even after about 2 hours, the expected gel-like thickened consistency was not obtained and the mixture was almost at the same consistency as before heat treatment or just subtly thicker, too thin and watery for cooking. This effect was observed in both replicates of the experiment.

Conclusion: Without wishing to be bound by theory, the result observed herein utilized a higher concentration of protein in the flour, accordingly the interaction between the protein and gellan gum prevented the gum from hydrating. This means the protein-gum interaction prevented the water-gum interaction from forming, preventing the hydration of the gum and its thickening/gelling effect in the matrix cl Example 8

### Ready to Drink Beverage

In this study, the objective is to formulate a nutritious beverage with a short clean list of ingredients using Euglena protein flour as the protein source.

Method and Materials: A chocolate beverage formulation was developed and Euglena protein flour, as the only protein source, at different percentages (3, 5, 7, 9% w/w final product) were tested and the sensory profile (aroma, taste, mouthfeel) of the beverage was evaluated.

All the dry ingredients (protein flour, cocoa powder) and liquid ingredients (maple syrup and water) are weighed and combined in a glass container with a tight lid (See Table 20). The mixture is vortexed or stirred using a stir bar for about 2 minutes until all the dry ingredients are dispersed and no clumps are observed. The beverage is homogenized for 2 minutes using a handheld (stand) homogenizer (i.e. OMNI International GLH-01) at speed setting 6 which is equivalent to 250,000 rpm. The homogenized beverage then is heated in a water bath at 80° C. for 12 minutes. This time-temperature combination was achieved after a few trials to identify the appropriate combination for the glass container, containing the beverage, to reach 72° C. for 15 seconds (milk pasteurization standard). The beverage is then cooled for 3 hours and tested for sensory profile.

TABLE 20
Ready to drink beverage

| Ready to Drink Beverage | % Ing. |
|---|---|
| Water | 69.50 |
| Maple Syrup (Fortune Farms, Product of Canada) | 15.00 |
| Euglena Protein-Rich Flour | 7.00 |
| Euglena B-Glucan Isolate | 5.00 |
| Euglena B-Glucan Isolate RTG | 2.00 |
| Cocoa Powder (No Name) | 1.50 |
| Total | 100.00 |

Results and Discussion: The above drink formulation with the Euglena products has 5.8 grams of Euglena protein/serving, 1.7% Euglena oil per serving, and 20.8 beta-glucan per serving. A serving size is 200 mL or 250 mL.

In terms of taste profile, at the above concentration of Euglena protein flour results in a subtle marine off-notes, which is not as noticeable as off-notes from higher concentrations. Euglena beta-glucan isolate was used in the formulation for its immune boosting effects. Euglena beta-glucan isolate RTG was used in the formulation to replace gum inclusion which is commonly used in beverage products for their imparted viscosity, mouthfeel and stability.

### Example 9

### Powdered Beverages

Powdered Beverage Overview: In this study, the objective is to formulate a nutritious, powdered, beverage with a clean list of ingredients using Euglena protein flour as the protein source.

Powdered Beverage Issues and Solutions

Clumping: Euglena protein beverage contains pea protein, euglena flour, pregelatinized starch, masker, flavours and sugar. This blend forms large clumps upon addition into the cold water. Tables 21 and 22 have a reduced level of pregelatinized starch and Ticaloid 620 (Locust bean gum and Xanthan gum mix from TIC) added to the formula to achieve a (current market) powdered beverage level of viscosity, without clumps.

Marine Flavour Masking: A variety of natural flavour maskers and flavours were tested throughout the development of the powdered beverage. Table 21 and 22 reflect the flavours and maskers best suited to eliminate any marine flavours.

TABLE 21
Contents of the Mango Flavoured Powdered Protein Beverage

| Mango flavoured powdered protein | | |
|---|---|---|
| shake | Amount | Ingredient % |
| Ingredient Name | (g) | (dry powder) |
| Pea Protein concentrate (Puris) | 12.6 | 28 |
| Euglena Protein Flour | 5 | 11.1 |
| Organic Agava | 10 | 22.2 |
| Pregelatinized starch | 8 | 17.8 |
| Mango flavour | 6 | 13.3 |
| Masker 2 | 3 | 6.7 |
| Guar gum + Gum Acacia+ Xanthan | 0.3 | 0.7 |
| Lemon Flavor | 0.1 | 0.2 |
| Water | 240 | |
| Masker 1 | 0.05 | 0.1 |
| Total Solids | 45 | |
| Total | 285 | 100.0 |
| Protein Content | 12.6 | |

TABLE 22
Contents of the Chocolate Flavoured Powdered Protein Beverage

| Chocolate flavoured powdered protein shake | Amount | Ingredient % |
|---|---|---|
| Ingredient Name | (g) | (dry powder) |
| Pea Protein concentrate | 15 | 30 |
| Euglena Protein Flour | 5 | 10 |
| Organic Agave | 12 | 24 |
| Cocoa powder (Dark Unsweetened) | 5 | 10 |
| Pregelatinized starch | 4.7 | 9.4 |
| Natural dark chocolate flavour | 2.5 | 5 |
| Natural vanilla flavour | 2.5 | 5 |
| NAT FL Modulasense Type | 2.5 | 5 |
| Masker 1 | 0.5 | 1 |
| Guar gum + Gum Acacia + Xanthan | 0.3 | 0.6 |
| Water | 240 | |
| Total solids | 50 | |
| Total (solid + water) | 290 | 100 |
| Protein Content | 14.5 | |

Method and Serving Suggestions

Consumer method: Consumer to receive a premixed package with multiple servings and a serving size scoop. They are then to prepare according to Table 23.

TABLE 23
Single Serve Preparation Instructions
Preparation Instruction for one serving (45-50 g)

Add a single serving of powdered beverage into 1 cup of ice-cold water
Shake the content vigorously and Enjoy!

Serving Suggestion/Future applications: Table 21 and 22 has been tested with oat milk and 2% milk, the results were a thicker and more indulgent prepared beverage. Thus showing the versatility of the powdered beverage and the possibility for other applications (smoothies, baking . . . ) to be considered during future powdered beverage work. Other milk or dairy alternatives suitable for the beverage and will be tested during consumer testing include, but not limited to, soy milk, soy milk blends, almond milk, lactose free milk, coconut milk, cashew milk and other

Conclusion and future efforts: Noblegen's current powdered beverage has overcome texture and flavour obstacles. The current flavours and formula are a viable competitor to current market products. A single serving offers 12.6 to 14.5 g of protein per serving in an easy to prepare application. Future powered beverage efforts will be made to decrease the amount of sugar and to add additional flavours to the current 2. Work on a liquid beverage is also a consideration.

### Example 10

### Euglena Based Protein Bars

The objective of this study was to include Euglena protein flour in a bar formulation (see Table 24) to replace part of the protein source, as well as to give a nutritional profile to the bar.

TABLE 24
Ingredient list of Euglena protein flour bar formulation

| Ingredient | Percentage (%) in formulation |
|---|---|
| Dates | 27.65 |
| Agave Syrup | 13.53 |
| Natural Peanut Butter | 11.18 |
| Coconut Oil | 2.94 |
| Rolled oats | 7.65 |
| Almonds | 7.06 |
| Euglena Protein Flour | 30.00 |
| Total | 100.00 |

To make the Euglena bars, heat an oven to 350° F. to toast almonds and oats for 10 minutes or until just golden, allow to cool. After cooling, rough chop almonds in a food processor and combine the dry ingredients in a mixing bowl, stir to combine and set aside. In a food processor, process the dates until a dough ball consistency forms, about 1 minute. Combine the wet ingredients together (agave syrup, peanut butter, coconut oil, dates) into a small double boiler and heat until the dates are easily incorporated into the wet, remove from heat. Add the wet ingredients into the dry ingredients using a spatula until well combined. Transfer mixture into a wax paper lined, appropriately sized bar mold and use a spatula to press dough firmly into the mold until uniformly distributed and level. Wrap bar in wax paper and place into refrigerator to set.

Results and discussion: The present formulation was developed by first testing 6.25% of Euglena flour and 6.25% beta-glucan isolate. Percentages of each were increased to 8% and 8.5%, respectively, followed by 10% and 7%, respectively. Formulations were also tested wherein beta glucan isolate was omitted from the recipe and the Euglena flour was increased to 12.5% 15%, 20%, 25%, and 30%. Beta-glucan isolate was reintroduced to test the moisture holding capacity within the bar at 5% and 25% Euglena flour.

The following describe previous formulations testing: Maple Syrup, Agave Syrup, Tapioca and Golden Strap Molasses, Agave and Tapioca Syrup, Beta glucan Isolate, Ready to gel (RTG)-Wet Gel, and Flavour Masking

Maple Syrup: Maple syrup was the first binder/sweetener used within the bar which comes from the original recipe. This worked well, provided enough moisture to the bar and presented good flavor with sweetness, but was not an ideal binder. Also, the shelf life was low. Maple Syrup has been used in all Euglena flour formulations (listed above, 6.25%-30%).

Agave Syrup: Agave Syrup was used because it had more viscosity than maple syrup, which could potentially have better binding properties. This was observed to be the case. Additionally, Agave Syrup has a lower glycemic index than Maple Syrup, providing increased nutritional value for longevity of energy. This bar included Euglena flour at 15%, 20%, 25% and 30%.

Tapioca and Golden Strap Molasses: This bar was a 30% Euglena flour inclusion. The Tapioca and Golden Strap Molasses mix was tested as a binder, which was expected to increase moisture while acting as a binder. This did not turn out, as the moisture content was too low in the formula when equal quantities of Tapioca and Golden Strap Molasses were used. The dry ingredients were insufficiently moisturized and the resulting texture was lumpy, difficult to work with and had poor flavor. This bar included Euglena flour at 30%.

Agave and Tapioca syrup: Agave and Tapioca syrup mix at a ratio of 50:50 was used. While blending the wet and dry it was noted the mixture was substantially drier than using the Agave alone. This was likely due to the fact that Tapioca syrup is very viscous and does not contain a lot of moisture. Before combining the bar together, the formula was adjusted to a 60:40 Agave:Tapioca split. All ingredients were added to the food processor to evenly distribute the new ratios before assembling the bar. The food processor-blended-bar demonstrated a greater crumbling vs the original packed bar due to the greater surface inclusion of the finely chopped ingredients. This bar included Euglena flour at 30%.

Beta glucan Isolate (BGI): The initial bars were made utilizing BGI and Euglena flour each at 6.25%. The percentage of BGI was increased, trying all of the rates listed above, with little noticed change. A final bar was combined with BGI omitted and there was no noticeable difference (i.e. mouth feel, taste). The percentage of Euglena flour was increased to gain a higher protein content in the bar formulation. Most recently, BGI is added to the bar formulation to determine if it would assist with holding moisture longer within the bar to increase shelf life. This was done utilizing 5% BGI and 25% Euglena flour with Agave syrup as a binder.

Ready To Gel (RTG)-Wet Gel: Ready to Gel (RTG) bar was initially made using 3.29% RTG, 30% Euglena flour, and maple syrup as a binder. The RTG was included in wet form to attempt to assist with moisture content of the bar. Ideally, the bar requires a 60:40 wet to dry ingredients. It is thought that the RTG would increase the moisture ratio of the wet:dry ingredients of the bar overall, as well as adding moisture to increase shelf life. However, it was noted that the bar was drier in appearance and was more crumbly and flaked when cut. It appeared that the RTG wet gel did not hold moisture as expected in terms of appearance, but increased the moisture content and improved the mouth feel of the bar.

Additional formulations will test different naturally derived flavour maskers to reduce the undesirable Euglena flavours, different binders such as brown rice syrup, malted barley syrup, malt extract and oat extract blend to optimize moisture content, consistency, and flavor and preservatives, for example Potassium Sorbate, to increase shelf life of the bars.

### Example 11

### Additional Examples of Euglena Based Protein Bars

Overview: Bar prototypes were created to showcase Euglena in a high protein sweet application. Bars were to have/be: 10 grams or more per serving, 5 grams coming from Euglena, Vegan, Gluten Free, Sugar content less or at par with protein content (not common in most protein rich bars), Euglena having the highest inclusion percentage.

All ingredients in the bar were chosen for either its nutrition profile, texture/mouthfeel properties, flavour and/or a combination of those properties.

Challenges and obstacles: The majority of the work on the bars went into perfecting the flavour and mouthfeel. The first few formulas had an unappealing euglena flavour and a powdery mouthfeel. Natural flavours that diminish the euglena flavour where added to the formula as well as, low sugar binding agents. Together these 2 additions, and some changes in the inclusion % of the ingredients, helped to form the current bar formulas seen in Tables 25 thru to 30.

Current Bar Formulas:

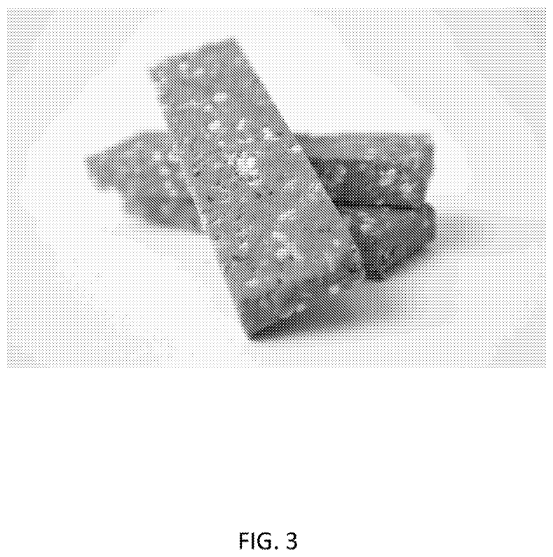

Coconut Citrus Bar (FIG. 3): The following is an example of the ingredients, and process involved in making the Coconut Citrus Bar, which gives an example of the Euglena protein flour in a sweet protein bar application. Citrus is an ideal flavour for an Euglena based bar as it naturally tones done any euglena off notes.

TABLE 25
Contents of the Coconut Citrus Bar

| Ingredients | Inclusion (%) |
|---|---|
| Euglena Flour | 16.8 |
| Brown Rice Syrup | 15.4 |
| Cocoa Butter | 9.8 |
| Pea Protein | 8.4 |
| Natural Flavour Extracts | 7.9 |
| Peanut butter | 7.9 |
| Unsweetened Coconut, shredded | 7.0 |
| Date Paste | 5.6 |
| Tapioca Syrup | 5.0 |
| Vegetable Glycerine | 4.2 |
| Cane Sugar | 2.8 |
| Ground oats | 2.8 |
| Ground Flax seed | 2.8 |
| Expeller pressed canola oil | 2.2 |
| Inulin Powder | 2.0 |
| Quinoa Puffs | 1.3 |
| Salt | 0.1 |

TABLE 26
Recipe Instructions for Coconut Citrus Bars

| | |
|---|---|
| 1. | Toast coconut at 375 F. for 9-10 minutes or until golden. |
| 2. | Place oats in the bowl of a food processor, pulse until oats resemble |
| | a whole grain flour consistency (medium ground thickness) |
| 3. | Sift flour, pea protein, ground flax seed, inulin powder, salt and |
| | quinoa puffs into the oats, pulse until combined. |
| 4. | Melt together brown rice syrup, cocoa butter, peanut butter, date |
| | paste,tapioca syrup, vegetable glycerine, cane sugar, and expeller |
| | pressed canola oil. |
| 5. | Pour wet mixture into the food processor with the oats/flour mixture |
| | and pulse until it comes together. |
| 6. | Add toasted coconut to the food processor, pulse until evenly |
| | combined. |
| 7. | Press into a wax paper lined container. Refrigerate for 30 minute |
| | before slicing. |

In this bar application, there is an 16.8% Euglena protein flour inclusion, which adds 5.22 g of protein to the bar and 2.2. g of beta-glucan. The total protein content in the bar is 10.95g and total sugar content 10.56g.

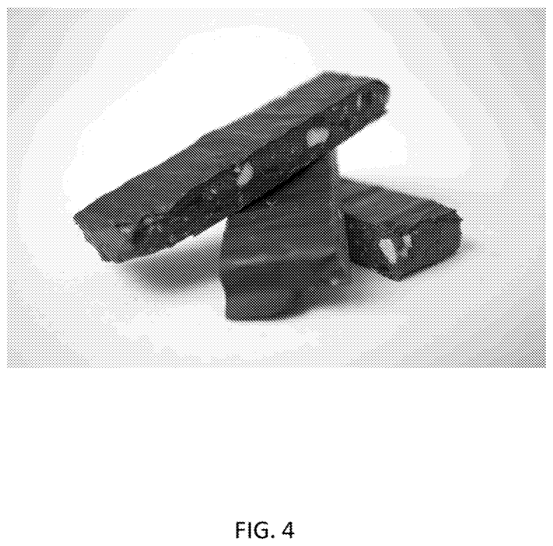

Dark Chocolate, Almond and Cranberry Protein Bar (FIG. 4)

TABLE 27
Contents of the Dark Chocolate,
Almond and Cranberry Protein Bar

| Ingredients | Inclusion (%) |
|---|---|
| Euglena Protein Rich Flour | 15.9 |
| Brown Rice Syrup | 11.9 |
| Natural Peanut Butter | 7.1 |
| Cocoa Butter | 5.9 |
| Pea Protein | 5.9 |
| Date Paste | 5.9 |
| Dried Cranberries | 4.7 |
| Toasted Almonds | 4.7 |
| Vegan Dark Chocolate | 4.7 |
| Tapioca Syrup | 4.5 |
| Natural Flavours | 4.3 |
| Cocoa Paste | 3.6 |
| Cocoa Powder | 3.6 |
| Vegetable Glycerin | 3.6 |
| Expeller Pressed Canola Oil | 2.4 |
| Cane Sugar | 2.4 |
| Ground Flax Seed | 2.4 |
| Ground Oats | 1.9 |
| Inulin Powder | 1.7 |
| Pink Salt | 0.1 |

The following is an example of the ingredients, and process involved in making the Dark Chocolate, Almond and Cranberry Protein Bar. The bitterness that comes from dark chocolate helps to reduce the amount of bitterness that can come from an Euglena aftertaste. A small amount of natural lemon flavour was added to the formula to help offset any Euglena flavor.

TABLE 28
Instructions for Dark Chocolate Almond Cranberry Bar

| | |
|---|---|
| 1. | In the bowl of a stand mixer sift together the Euglena flour, |
| | pea protein, dried cranberries, chopped almonds, cocoa powder, |
| | ground flax seeds, ground oats, inulin powder and salt. Mix |
| | with whisk attachment until combined. |
| 2. | Switch to paddle attachment. |
| 3. | In a double boiler melt together brown rice syrup, peanut butter, |
| | date paste, cocoa butter, cocoa paste, vegetable glycerin, |
| | expeller pressed canola oil, tapioca syrup and cane sugar. Place |
| | wet ingredients in a food processor and pulse until smooth. |
| 4. | Add wet ingredients to dry ingredients, mix at speed level 4 until |
| | a uniform dough is formed. |
| 5. | Transfer dough to a wax paper lined, appropriately sized bar mold |
| | and use a spatula to press dough firmly into the mold until |
| | uniformly distributed and level. Let cool. |
| 6. | Melt dark chocolate over a double boiler until smooth. Pour and |
| | spread evenly over bars, let cool. Once cooled and chocolate has |
| | hardened, slice into 60 g bars. |

In this bar application, there is an 15.90% Euglena protein flour inclusion, which adds 4.92 g of protein to the bar and 2.40 g of beta-glucan. The total protein content in the bar is 11.20 g and total sugar content 10.96g.

Peanut Butter Chocolate Chip Cookie Dough Bar

The following is an example of the ingredients, and process involved in making the Peanut Butter Chocolate Chip Bar using Euglena flour. This provides a tasty, high protein and cleaner label protein bar example.

TABLE 29
Contents of the Peanut Butter Chocolate Chip Cookie Dough Bar

| Ingredients | Inclusion (%) |
|---|---|
| Crunchy peanut butter | 22.00 |
| Date paste | 22.00 |
| Protein Rich Euglena Flour | 11.00 |
| Pea Protein | 11.00 |
| Vegan Dark Chocolate | 10.38 |
| Cocoa Butter | 10.38 |
| Natural Flavours | 3.88 |
| Expeller Expressed Canola Oil | 3.88 |
| Vegetable Glycerin | 3.88 |
| Tapioca Syrup | 2.20 |

TABLE 30
Instructions for Peanut Butter Chocolate Chip Cookie Dough Bar

| | |
|---|---|
| 1. | In the bowl of a stand mixer sift together the euglena flour and pea |
| | protein, mix together until combined. |
| 2. | In a double boiler, melt together the crunchy peanut butter, date |
| | paste, cocoa butter, expeller pressed canola oil, vegetable glycerin |
| | and tapioca syrup. |
| 3. | Add wet ingredients to dry ingredients mix at speed level 4 until a |
| | uniform dough is formed |
| 4. | Transfer dough to a wax paper lined, appropriately sized bar mold |
| | and use a spatula to press dough firmly into the mold until |
| | uniformly distributed and level. Let cool. |
| 5. | Melt dark chocolate over a double boiler until smooth. Pour and |
| | spread evenly over bars, let cool. Once cooled and chocolate has |
| | hardened, slice into 60 g bars. |
| 6. | This product has 11% Euglena protein flour inclusion, with 3.5 g |
| | protein from Euglena protein flour, and a total protein content in |
| | the bar of 12.5 g. |

Future work: To date euglena has been used primarily in pressed bar form, work will continue to grow the bar category to include: Baked bars, granola bars, energy balls and crisp bars.

Euglena has already been incorporated into baked goods which shows good promise for the baked bars market Work will also be done to reduce the sugar content and increase the protein content of the current bars and any pressed bars moving forward.

### Example 12

### Fresh Noodles

Introduction: Noodle and pasta work has been focused on creating a protein rich euglena noodle. Noodle types in development are; Basic pasta, Gluten free pasta, Vegan pasta, Buckwheat (soba noodles), 30% Vegan Euglena noodles (still in research phase).

Current Formulas

The following Tables 31 to 34 are the current formulas for each dough. Testing was conducted in a consumer style kitchen with a Kitchen-Aid stand mixer and pasta roller attachments. Each dough was rolled to level 4 thickness and cut into linguine. Testing was done by establishing a control formula for each of the below noodle types. Then the main flour(s) type were reduced in increments from 5% to 30%, depending on the dough, and replaced with Protein Rich Euglena Flour until an ideal inclusion percentage was found.

Basic Egg Pasta Dough

TABLE 31
Ingredients in the Basic Egg Pasta Dough

| Ingredients | Grams | % |
|---|---|---|
| All purpose flour | 55 | 37.4 |
| Eggs | 55 | 37.4 |
| Oil | 5 | 3.4 |
| Salt | 2 | 1.4 |
| Protein Rich | 30 | 20.4 |
| Euglena Flour | | |
| Total | 147 | 100.0 |
| Protein | | 19.49 |

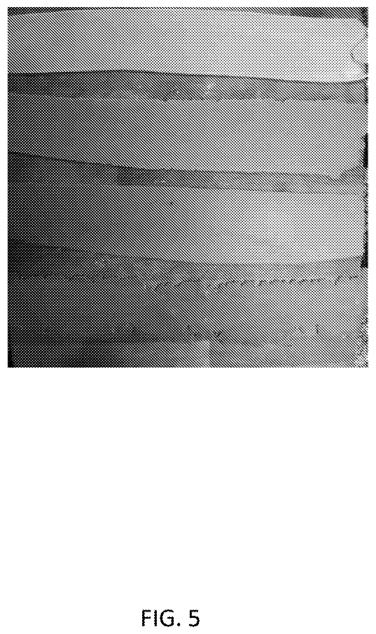

At the 20% inclusions, the dough was slightly more difficult to knead and roll, but it was still workable by hand. The use of commercial equipment will help element this issue. Cooking time was similar to control at 4 minutes and the euglena flavour was noticeable but not off putting. The cooked texture was denser than the control but still palatable. FIG. 5 shows the rolled egg dough, as you can see the higher the level of euglena the dryer and more delicate the dough becomes.

Gluten Free Pasta Dough

TABLE 32
Gluten Free Pasta Dough Ingredients

| Ingredients | Grams | % |
|---|---|---|
| Gluten free Flour | 70 | 47.1 |
| Egg | 55 | 37.0 |
| Protein Rich Euglena | 15 | 10.1 |
| Flour | | |
| Oil | 5 | 3.4 |
| Salt | 2 | 1.3 |
| Xanthan gum | 1.5 | 1.0 |
| Total | 148.5 | 100.0 |
| Protein | | 13 |

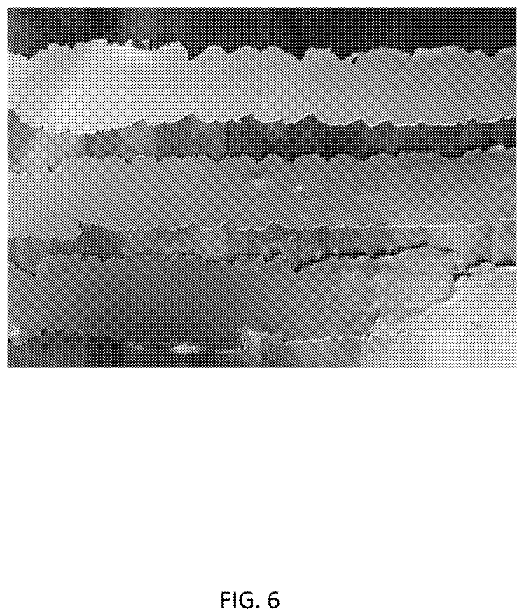

10% inclusion was the highest inclusion rate achievable with a gluten free flour. No difference in the raw dough texture was noted in comparison to the control, it was also successfully cooked within the same time as the control, 5 minutes. The cooked dough had a very similar texture to the control with a small amount of euglena after taste. As you can see in FIG. 6 the gluten free dough is naturally denser and the highest workable inclusion of euglena was 20%.

Eggless Pasta Dough

TABLE 33
Ingredients in the Eggless Pasta Dough

| Ingredients | Grams | % |
|---|---|---|
| All purpose flour | 68 | 43.3 |
| Water | 50 | 31.8 |
| Olive oil | 5 | 3.2 |
| Salt | 2 | 1.3 |
| Protein Rich | 32 | 20.4 |
| Euglena Flour | | |
| Total | 157 | 100.0 |
| Protein | | 14 |

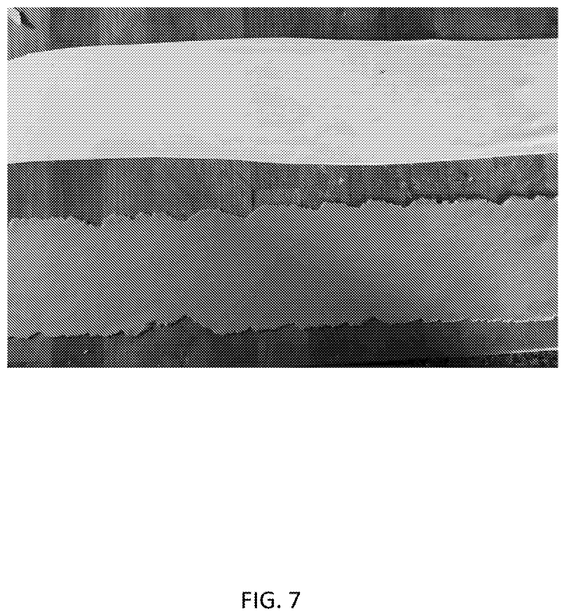

A 20% inclusion rate was ideal for the eggless pasta dough. Similar to the basic egg pasta dough (Table 31) it became increasingly difficult to roll with the higher inclusion of euglena, but with commercial pasta equipment this would be less of a problem. Compared to the control this formula took an extra minute to cook and had a low to medium euglena flavour. The cooked texture was more dense than control, but only slightly and this could be corrected with a slightly longer cooking time. As FIG. 7 shows, the 20% euglena inclusion for an eggless formula is dense in comparison to the control, but still rolled with only tearing on the edges.

Buckwheat/Soba Noodles

TABLE 34
Ingredients in Buckwheat/Soba Noodles

| Ingredients | Grams | % |
|---|---|---|
| Buckwheat flour | 65 | 25.2 |
| All Purpose Flour | 62 | 24.0 |
| Warm water | 100 | 38.8 |
| Protein Rich | 26 | 10.1 |
| Euglena Flour | | |
| Salt | 5 | 1.9 |
| Total | 258 | 100.0 |
| Protein | | 15 |

Buckwheat noodles are naturally more dense than all purpose flour based noodles. Due to this the highest inclusion of euglena that is workable by hand is 10%. Anything higher than 10% would rip when fed through the pasta roller. The cooking time did not differ from the control. The overall flavour and texture was similar to the control. The natural flavour of buckwheat helped to eliminate most of the euglena flavour notes.

Future Work: Moving forward the focus of paste/noodle should be on a 30% euglena noodle with 20 g of protein preserving. Natural maskers could be used to reduce the euglena flavour. A key finding from the testing process was that the euglena flour produces a denser dough. Further equipment might be needed in order to work with a 30% euglena formula, as well as, other texture optimizing ingredients. Table 34 (Buckwheat/Euglena noodles) were tested once tossed in a ginger, garlic and miso sauce. There was no euglena after taste but the noodle produced a unique flavour that proves there is serious potential in the noodle market for euglena.

With further testing and equipment euglena noodles could be successfully formed into a variety of different shapes (penne, rotini, spaghetti) or used for stuffed pasta dishes (ravioli, cannelloni).

There is also the possibility that euglena could also be used in different types of noodles: Udon, Ramen, Glass noodles, Yakisoba

### Example 13

### Mac and Cheese

Currently an Euglena Macaroni and Cheese prototype is in the development phase. The aim is to create a high protein, vegan, and gluten free product. It will be packaged in a box containing a pre measured portion of dried noodles and an envelope of powdered sauce. The consumer will be responsible for cooking the pasta and hydrating the sauce with either water or milk (dairy or dairy free) of their choice. The protein goal is 12 -15 grams of protein per serving, half of which should be coming from Euglena.

Challenges: Taste and texture are the 2 main challenges being addressed during work on this prototype.

Taste: The natural flavour of Euglena flour has savoury notes, by using other flavours that compliment Euglena and work to develop the “cheesy” flavours we are trying to obtain will help us diminish any off putting flavours.

Texture: Stabilizers and texture will can be used to help obtain an ideal texture for the finished sauce.

Table 35 is an example of work being done on a classic “Cheddar Cheese Macaroni and Cheese”.

TABLE 35
Macaroni and Cheese Current formula

| Ingredients | “Box” (grams) | % |
|---|---|---|
| Natural flavours | 15.25 | 5.17 |
| Euglena flour | 10 | 3.39 |
| Yeast, nutritional, | 4 | 1.36 |
| Pea protein | 5 | 1.69 |
| Dried, minced Garlic | 3 | 1.02 |
| Sunflower lecithin | 1.5 | 0.51 |
| Tapioca Starch | 2 | 0.68 |
| Red Chili Flakes, Crushed | 0.5 | 0.17 |
| Quinoa macaroni pasta | 170 | 57.63 |
| Oat milk | 125 | 42.37 |
| Total | 295 | 100.00 |
| Protein (approx.) | 11 | |

### Example 14

### Euglena Extrusion Example

Introduction: Extruded products are a form of mass food processing that is used to make a variety of different foods, such as pasta, cereals, bread, textured vegetable protein (TVP), and ready to eat snacks. Depending on the processes used, products can be puffed, such as a ready to eat snack and/or textured, such as TVP. In this example, Euglena protein flour was extruded with different ratios of pea protein, rice flour, as well as with or without the use of maskers. This highlights the Euglena flour as a possible ingredient replacer or as a functional ingredient for extrusion.

Materials and methods: Several different formulations using Euglena flour as an ingredient were tested, where the inclusion levels of Euglena flour, pea protein and use of maskers was varied (Table 36). Rice flour is added to help bind the mixture together while mixed tocopherols are added to preserve freshness. Two different maskers were also testing in the formulations, to help generate a more neutral flavor profile to the final products.

Products were extruded using standard methods for dry extrusion and for a residence time of 0.5 minutes, but can be extruded up to 3 minutes. Final products have an expansion ratio of 0.3-0.4 g/cc.

TABLE 36
Extruded product formulations tested. Pea protein, Euglena protein,
and masker inclusion were varied in the different versions.

| Ingredient | Version | Version | Version | Version | Version | Version |
|---|---|---|---|---|---|---|
| Name | 1 (%) | 2 (%) | 3 (%) | 4 (%) | 5 (%) | 6 (%) |
| Pea Protein; | 65.00 | 55.00 | 47.00 | 55.00 | 55.00 | 20.00 |
| 80% protein | | | | | | |
| Euglena | 10.00 | 20.00 | 28.00 | 20.00 | 20.00 | 55.00 |
| Protein Flour; | | | | | | |
| 40% protein | | | | | | |
| Rice Flour | 24.85 | 24.85 | 24.85 | 23.85 | 23.85 | 23.85 |
| Mixed | 0.15 | 0.15 | 0.15 | 0.15 | 0.15 | 0.15 |
| Tocopherols | | | | | | |
| Masker 1 | 0.00 | 0.00 | 0.00 | 2.00 | 0.00 | 1.00 |
| Masker 2 | 0.00 | 0.00 | 0.00 | 0.00 | 1.00 | 0.00 |
| Total | 100.00 | 100.00 | 100.00 | 101.00 | 100.00 | 100.00 |

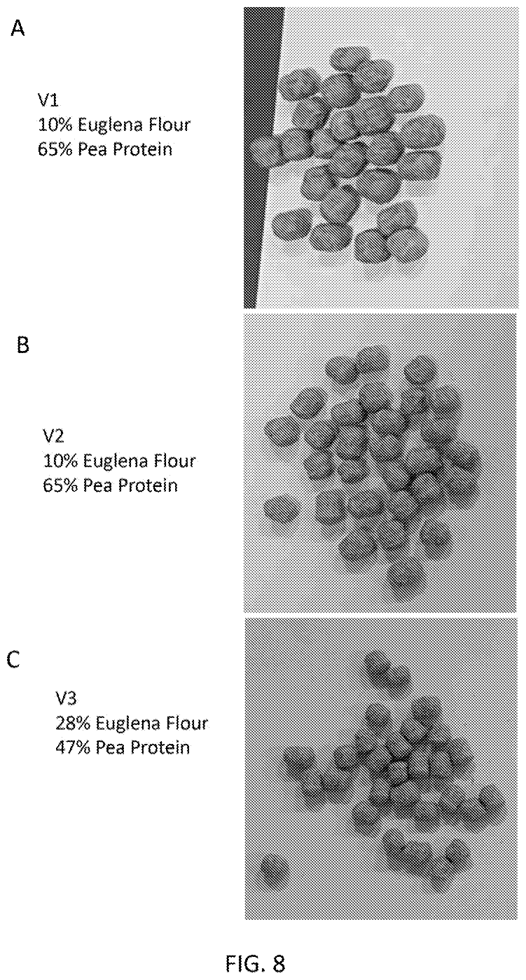

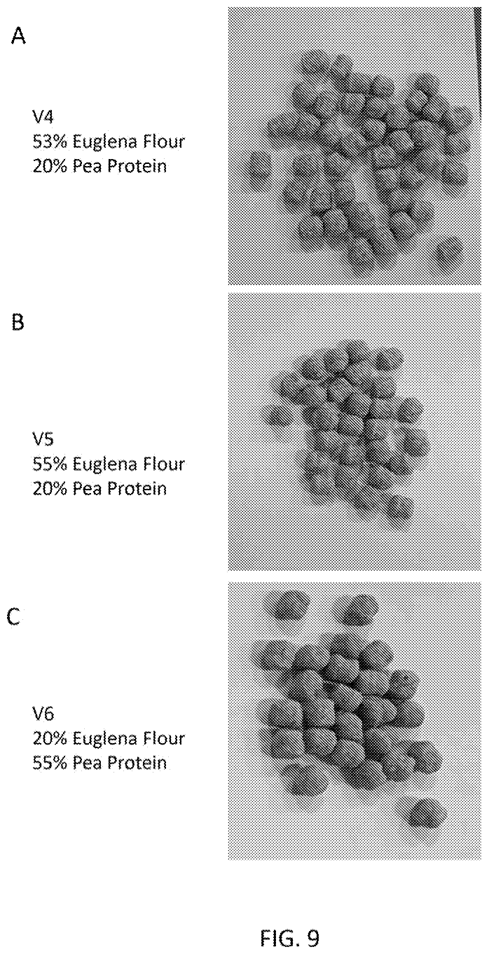

Results and discussion: Extruded products resulted in a small, crisp puff that is crunchy on the outside and firm on the inside. Similar results were seen between different ratios, with or without maskers (FIGS. 8 and 9). This demonstrated a successful extrusion and puff of a mixture with Euglena protein flour.

Effect of the maskers is initially used for product quality. Version 6 with the highest inclusion of masker eliminated the Euglena, marina flavour. Over storage time, maskers were shown to decrease off notes, perhaps by neutralizing the volatile compounds in the mixture.

Conclusion: Small round puffs were produced in this trial. Future formulations can vary the Euglena flour up 100% inclusion as a protein i.e. up to 75-80% in the formulation. Euglena protein concentrate and or Euglena Protein isolate could be used to replace the Pea protein, and or also replace the Euglena flour in the formulations. It is possible to have an all Euglena protein based formulation, with a mixture of Euglena protein rich flour, concentrate and isolate.

If a formation is mixed with another protein, other protein isolates than pea can be used. Such examples include, soy, corn, wheat, rice, beans, seeds, nuts i.e. almond protein, peanut protein, seitan, lentil, chickpeas, flaxseed, wild rice, quorn, chia seeds, quinoa, oats, fava beans, buckwheat, bulgar, millet, microalgae, yellow pea, mung bean, hemp, sunflower protein, legumes.

In addition, other starches then rice flour can be used, such as tapioca starch, rice starch, pea starch, corn starch, wheat starch, barley starch, sorghum starch, potato starch, sweet potato starch, turmeric starch, ginger starch, dioscorea starch, water chestnut starch, arrowroot starch, oat starch, banana starch, lentil starches, yellow pea starch, chickpea starch, mung bean starch, and amaranth starch.

Extruded puffs would make an excellent addition as a cereal, in cereal bars, granola bars, as a good protein source in salads as a salad topper, and incorporated into baked goods. A good use of the extruded puffs would be as a puff snack, and could be coated in powdered flavorings to be eaten as a ready made food snack.

If wet extrusion was used, it would further texturize the product and can expand the product list to others such as pasta, textured Euglena protein, plant based muscle analogues, and pulled meat alternatives. In wet extrusion, instead of puffing the product by expansion, the objective would be to texturize the mixture through different standard production methodology. One focus could be on generating a Euglena protein based TVP product, with different sized crumbles, or a material like a pulled pork or chicken, ¼ inch to 1 inch in size. It could also be used to generate an Euglena based soy curl. The goal of the meat alternatives would be to generate an alternative for any beef, pork, poultry, and seafood based products. In terms of seafood, it could be combined with seaweed to generate several different seafood applications, such as crab/lobster, shrimp, clams, scallops, calamari, smoked fished, dried seaweed snacks, and meat alternatives.

In conclusion, versions were successful in creating a puffed extruded product and with further optimization, increases in Euglena protein rich flour or Euglena protein are possible. Maskers helped stabilize the taste of the product over time.

### Example 15

### Euglena Protein Flour, Concentrate and Isolate Inclusion in a Variety of Food Products (Table 37)

TABLE 37
Food Table. Dots represent most likely form(s) used in food type,
but is not limited to said form, as seen in percentage values.

| FOOD TYPE | FLOUR | CONCENTRATE | ISOLATE | TECHNIQUE | PERCENTAGE |
|---|---|---|---|---|---|
| NON-DAIRY | | | | | |
| Milk (Euglena | • | • | • | Solubility | F: 10-15% |
| based) | | | | | C: 10-15% |
| | | | | | I: 5-20% |
| Casein | | • | • | Gelation | F: 10-15% |
| Replacement | | | | Foaming | C: 10-15% |
| | | | | Emulsification | I: 5-20% |
| Whip Cream | | • | • | Foaming | F: 1-10% |
| | | | | Emulsification | C: 1-10% |
| | | | | | I: 1-5% |
| Yogurt | | • | • | Gelation | F: 10-15% |
| | | | | Foaming | C: 10-15% |
| | | | | Emulsification | I: 5-20% |
| Protein Drinks | • | • | • | Solubility | F: 10-20% |
| | | | | | C: 10-20% |
| | | | | | I: 5-20% |
| Cheese | | • | • | Gelation | F: 10-15% |
| | | | | Foaming | C: 10-15% |
| | | | | Emulsification | I: 5-20% |
| Non-Dairy | • | • | • | Gelation | F: 1-5% |
| Creamer | | | | Foaming | C: 1 -5% |
| | | | | Emulsification | I: 1-15% |
| MEAT | | | | | |
| ANALOGUES | | | | | |
| Sausage | • | • | • | Emulsification | F: 40-45% |
| | | | | Foaming | C: 40-45% |
| | | | | Gelation | I: 50-80% |
| Pepperoni/cured | • | • | • | Emulsification | F: 40-45% |
| meat | | | | Foaming | C: 40-45% |
| | | | | Gelation | I: 50-80% |
| Chicken | • | • | • | Emulsification | F: 40-45% |
| | | | | Foaming | C: 40-45% |
| | | | | Gelation | I: 50-80% |
| Pork | • | • | • | Emulsification | F: 40-45% |
| | | | | Foaming | C: 40-45% |
| | | | | Gelation | I: 50-80% |
| Bacon Bits | • | • | • | Emulsification | F: 40-60% |
| | | | | Foaming | C: 40-60% |
| | | | | Gelation | I: 50-90% |
| Beef | • | • | • | Emulsification | F: 40-45% |
| | | | | Foaming | C: 40-45% |
| | | | | Gelation | I: 50-80% |
| Tofu Replacement | • | • | • | Emulsification | F: 10-15% |
| | | | | Foaming | C: 10-15% |
| | | | | Gelation | I: 5-20% |
| Ground Beef | • | • | • | Emulsification | F: 40-45% |
| | | | | Foaming | C: 40-45% |
| | | | | Gelation | I: 50-80% |
| Bacon/Jerky | • | • | • | Emulsification | F: 40-60% |
| | | | | Foaming | C: 40-60% |
| | | | | Gelation | I: 50-90% |
| Beef Patty | • | • | • | Emulsification | F: 40-45% |
| | | | | Foaming | C: 40-45% |
| | | | | Gelation | I: 50-80% |
| Ham | • | • | • | Emulsification | F: 40-45% |
| | | | | Foaming | C: 40-45% |
| | | | | Gelation | I: 50-80% |
| Turkey | • | • | • | Emulsification | F: 40-45% |
| | | | | Foaming | C: 40-45% |
| | | | | Gelation | I: 50-80% |
| SNACKS | | | | | |
| Potato Chips | • | • | • | Emulsification | F: 10-15% |
| | | | | Foaming | C: 10-15% |
| | | | | Gelation | I: 5-20% |
| Crackers | • | • | • | Emulsification | F: 10-15% |
| | | | | Foaming | C: 10-15% |
| | | | | Gelation | I: 5-20% |
| Bars | • | • | • | Emulsification | F: 5-20% |
| | | | | Foaming | C: 5-20% |
| | | | | Gelation | I: 10 -30% |
| Nut Butters | • | • | • | Emulsification | F: 20-30% |
| | | | | Foaming | C: 20-30% |
| | | | | Gelation | I: 15-35% |
| Pretzels | • | • | • | Emulsification | F: 10-15% |
| | | | | Foaming | C: 10-15% |
| | | | | Gelation | I: 5-20% |
| Euglena Snack | | • | • | Emulsification | C: 20-30% |
| (Similar to Dried | | | | Foaming | I: 15-35% |
| Seaweed) | | | | Gelation | |
| REFRIGERATED | | | | | |
| FOODS | | | | | |
| Gelatin | | • | • | Gelation | F: 1-10% |
| Replacement | | | | Foaming | C: 1-10% |
| | | | | Emulsification | I: 1-10% |
| Sauces/Condiments | • | • | • | Gelation | F: 5-15% |
| | | | | Foaming | C: 5-15% |
| | | | | Emulsification | I: 5-25% |
| Juices | | • | • | Solubility | C: 5-15% |
| | | | | | I: 5-20% |
| Soups/Chowders | • | • | • | Viscosity | F: 5-15% |
| | | | | Emulsification | C: 5-15% |
| | | | | Water | I: 5-25% |
| | | | | Adsorption | |
| DRY/READY | | | | | |
| TO EAT | | | | | |
| Cereal | • | • | • | Emulsification | F: 10-15% |
| | | | | Foaming | C: 10-15% |
| | | | | Gelation | I: 5-20% |
| Pasta | • | • | • | Emulsification | F: 10-15% |
| | | | | Foaming | C: 10-15% |
| | | | | Gelation | I: 5-20% |
| Crisps | • | • | • | Emulsification | F: 40-45% |
| | | | | Foaming | C: 40-45% |
| | | | | Gelation | I: 50-80% |
| Cookies | • | • | • | Emulsification | F: 5-15% |
| | | | | Foaming | C: 5-15% |
| | | | | Gelation | I: 5-20% |
| Puffs | • | • | • | Emulsification | F: 30-40% |
| | | | | Foaming | C: 30-40% |
| | | | | Gelation | I: 35-50% |
| Oatmeal | • | • | • | Emulsification | F: TBD % |
| | | | | Foaming | C: TBD % |
| | | | | Gelation | I: TBD % |
| BAKED GOODS | | | | | |
| Breads/Bagels/ | • | • | • | Emulsification | F: 5-10% |
| Buns | | | | Foaming | C: 5-10% |
| | | | | Gelation | I: 5-10% |
| Cookies | • | • | • | Emulsification | F: 5-15% |
| | | | | Foaming | C: 5-15% |
| | | | | Gelation | I: 5-20% |
| Brownies | • | • | • | Emulsification | F: 10-20% |
| | | | | Foaming | C: 10-20% |
| | | | | Gelation | I: 5-15% |
| Dry Mixes | • | • | • | Emulsification | F: 10-20% |
| (Brownie/Cake) | | | | Foaming | C: 10-20% |
| | | | | Gelation | I: 5-15% |
| Cake | • | • | • | Emulsification | F: 10-20% |
| | | | | Foaming | C: 10-20% |
| | | | | Gelation | I: 5-15% |
| Muffins | • | • | • | Emulsification | F: 10-15% |
| | | | | Foaming | C: 10-15% |
| | | | | Gelation | I: 5-20% |
| Pizza Dough | • | • | • | Emulsification | F: 5-I0% |
| | | | | Foaming | C: 5-I0% |
| | | | | Gelation | I: 5-10% |
| Flour (in general) | • | • | • | Emulsification | F: 25-50% (up to |
| | | | | Foaming | 100% flour, not |
| | | | | Gelation | tested for function) |
| | | | | | C: 10-20% |
| | | | | | I: 5-15% |
| FERMENTED | | | | | |
| PRODUCTS | | | | | |
| Kombucha | | • | • | Emulsification | F: 5-10% |
| | | | | Foaming | C: 5-10% |
| | | | | Gelation | I: 5-20% |
| | | | | Solubility | |
| Sauerkraut | | • | • | Emulsification | F: 5-10% |
| | | | | Foaming | C: 5-10% |
| | | | | Gelation | I: 5-15% |
| | | | | Solubility | |
| Kimchi | | • | • | Emulsification | F: 5-10% |
| | | | | Foaming | C: 5-10% |
| | | | | Gelation | I: 5-20% |
| | | | | Solubility | |
| Kefir | | • | • | Emulsification | F: 5-10% |
| | | | | Foaming | C: 5-10% |
| | | | | Gelation | I: 5-15% |
| | | | | Solubility | |
| Miso | | • | • | Emulsification | F: 5-10% |
| | | | | Foaming | C: 5-10% |
| | | | | Gelation | I: 5-20% |
| | | | | Solubility | |
| Pickles | | • | • | Emulsification | F: 5-15% |
| | | | | Foaming | C: 5-15% |
| | | | | Gelation | I: 5-20% |
| | | | | Solubility | |
| FROZEN FOODS | | | | | |
| Ice Cream | | • | • | Gelation | F: 10-15% |
| | | | | Foaming | C: 10-15% |
| | | | | Emulsification | I: 5-20% |
| Frozen Dinners | • | • | • | Emulsification | F: 10-40% |
| | | | | Foaming | C: 10-40% |
| | | | | Gelation | I: 5-45% |
| | | | | Solubility | |
| Cool Whip | | • | • | Gelation | F: 1-10% |
| | | | | Foaming | C: 1-10% |
| | | | | Emulsification | I: 1-10% |
| Cakes | • | • | • | Emulsification | F: 10-20% |
| | | | | Foaming | C: 10-20% |
| | | | | Gelation | I: 5-15% |
| Sauces | • | • | • | Emulsification | F: 5-15% |
| | | | | Foaming | C: 5-15% |
| | | | | Gelation | I: 1-20% |
| | | | | Solubility | |
| Frozen Pasta with | • | • | • | Emulsification | F: 15-30% |
| sauce | | | | Foaming | C: 15-30% |
| | | | | Gelation | I: 10-35% |
| | | | | Solubility | |
| Frozen Pizza | • | • | • | Emulsification | F: 10-30% |
| | | | | Foaming | C: 10-30% |
| | | | | Gelation | I: 5-35% |
| | | | | Solubility | |
| EGG | | | | | |
| REPLACEMENT | | | | | |
| Hard Boiled Egg | | • | • | Foaming | F: 10-40% |
| Type | | | | Gelation | C: 10-40% |
| | | | | | I: 5-45% |
| Powdered Eggs | • | • | • | Foaming | F: 10-40% |
| | | | | Gelation | C: 10-40% |
| | | | | | I: 5-45% |
| Liquid Eggs | | • | • | Foaming | F: 10-40% |
| | | | | Gelation | C: 10-40% |
| | | | | | I: 5-45% |
| Frozen Eggs | | • | • | Foaming | F: 10-40% |
| | | | | Gelation | C: 10-40% |
| | | | | | I: 5-45% |
| Salad Dressings | | • | • | Gelation | F: 1-10% |
| | | | | Foaming | C: 1-10% |
| | | | | Emulsification | I: 1-15% |
| Mayonnaise | | • | • | Gelation | F: 1-15% |
| (with/without | | | | Foaming | C: 1-15% |
| flavour) | | | | Emulsification | I: 1-20% |
| MISCELLANEOUS | | | | | |
| Gluten Free | • | • | • | Emulsification | F: 1-50% |
| Products | | | | Foaming | C: 1-50% |
| | | | | Gelation | I: 1-50% |
| | | | | Solubility | |
| Salad Bar Type | • | • | • | Emulsification | F: 5-25% |
| Products | | | | Foaming | C: 5-25% |
| | | | | Gelation | I: 1-30% |
| | | | | Solubility | |
| Textured | • | • | • | Emulsification | F: 10-40% |
| Vegetable Protein | | | | Foaming | C: 10-40% |
| | | | | Gelation | I: 5-45% |
| | | | | Solubility | |
| Soy Replacement | • | • | • | Emulsification | F: 10-40% |
| | | | | Foaming | C: 10-40% |
| | | | | Gelation | I: 5-45% |
| | | | | Solubility | |
| SEAFOOD | | | | | |
| Imitation | | • | • | Emulsification | C: 5-20% |
| Crab/Lobster | | | | Foaming | I: 5-25% |
| | | | | Gelation | |
| Clam Chowder | • | • | • | Viscosity | F: 5-20% |
| | | | | Emulsification | C: 5-20% |
| | | | | Water | I: 5-25% |
| | | | | Adsorption | |
| Fish | | • | • | Emulsification | C: 5-20% |
| | | | | Foaming | I: 5-25% |
| | | | | Gelation | |
| Sushi | | • | • | Emulsification | C: 5-20% |
| | | | | Foaming | I: 5-25% |
| | | | | Gelation | |
| PET FOODS | | | | | |
| Protein | • | • | • | Emulsification | F: 10-30% |
| Supplement | | | | Foaming | C: 10-30% |
| (Dry Pet Food) | | | | Gelation | I: 5-35% |
| Wet Cat/Dog | | • | • | Emulsification | F: 10-30% |
| Food | | | | Foaming | C: 10-30% |
| | | | | Gelation | I: 5-35% |

TABLE 38
Additional examples of future work with Euglena protein flour, concentrate and isolate

| PROTEIN TYPE | FUTURE USES | EXAMPLES |
|---|---|---|
| HIGH PROTEIN | Protein Rich | vegan broths to be used in the place of traditional |
| FLOUR | Broths | vegetable broths. Possible flavour ideas include: |
| | | Mushroom, Ramen, Chicken, Seafood and Beef. |
| | Crackers/Crisps | Euglena flour to produce a crisp or cracker with a |
| | | higher/equal protein content then those currently in |
| | | the healthy snacking market. |
| | Protein Salad | Salad toppers in the way of a protein crouton, |
| | Toppers | seasoned nut and seeds mixes, used to add extra |
| | | nutrients to salads and other meals. This is an |
| | | application of the extruded Euglena crisps seen in |
| | | Example 14. |
| | Protein Bites | Similar to the pressed bars but in bite size form. |
| | | Can be marketed as high protein or energy bites. |
| | Baked Goods | Vegan baked goods with an increased amount of |
| | | protein, can include dry mixes such as: Muffins, |
| | | Pancakes, Waffles, Cookies, Bars, Brownies, |
| | | Biscuits |
| | Protein Seasoning | A mixture of Euglena and spices to be used to top |
| | Mixes | popcorn, vegetables and other applications. |
| PROTEIN | Meat Analogues | The mixture of protein and oil in the concentrate |
| CONCENTRATE | | could help create a variety of meat analogues such |
| | | as: Sausage (any meat analogue or flavour), |
| | | Chicken, Cured meats, Tofu replacement. |
| | Seafood Analogues | The natural flavour of Euglena would be useful in |
| | | seafood analogies, such as: Shrimp, Smoked fish, |
| | | Dried seaweed snacks, crab/lobster, clams, |
| | | scallops, calamari, and meat alternatives.. |
| | Sauces | To add extra protein to different ready to use |
| | | sauces or ready to hydrate sauces such as Vegan |
| | | cheese sauce, and Curry sauces. Mayonnaise and |
| | | other dressings as well as the protein and oil of the |
| | | concentrate would be ideal. |
| PROTEIN | Dairy/Dairy | used for solubility, protein drinks for a thicker and |
| ISOLATE | Alternatives | high protein drink |
| | Breads and Doughs | which added the benefits of Euglena into breads |
| | | and doughs without altering the overall traditional |
| | | flavour i.e. Pizza doughs or Enriched breads |

Tables 37 and 38 highlight different functional uses of protein flour, protein concentrate and protein isolate from Euglena. The following describe various methods of quality control and the techniques as mentioned in Tables 37 and 38.

Emulsification Activity

5 mL oil, such as canola was added to 5 mL of 5% suspension of Euglena flour powder, and the mixture was homogenized for 3 mins. Emulsion was centrifuged for 5 mins at 500 rpm. The height of emulsion layer was measured using graduations on centrifuge tube.

$$EA = \frac{Height of the emulsified layer ( mm ) \times 100}{Height of the total contents in the tube ( mm )}$$

Emulsion Stability: Same procedure as emulsification activity but sample was heated to 80° C. in a water bath for 30 mins prior to centrifugation. The sample was then held under cool running water for 15 mins.

$$ES = \frac{Height of emulsified layer after heating ( mm ) \times 100}{Height of total contents in the tube ( mm )}$$

Foaming Capacity: 20mLs of 5% suspension was whipped at 1600 rpm for 5 mins. The mixture was poured into a 100mL graduated cylinder and foam volume was recorded.

$$FC = \frac{\begin{matrix}volume after whipping ( mL ) - \\ volume before whipping ( mL ) \times 100\end{matrix}}{Volume before whipping ( mL )}$$

Foaming Stability: 20 mLs of 5% suspension was whipped at 1600 rpm for 5 mins. The mixture was poured into a 100 mL grad. cylinder and foam volume was recorded after different time intervals ranging from 30sec to 60 min

$$FS = \frac{Foam volume at time t ( s ) \times 100}{Initial foam volume ( mL )}$$

Apparent Viscosity: Triplicate Euglena flour suspensions (45.0+/− 0.05 g/255 ml distilled water) were made up in 500-ml Pyrex beakers and mixed by a Servodyne mixer head at 800 rpm using a Servodyne mix controller for 20 s followed by a 5 min hydration period. Each mixture was poured into a 400-ml tall beaker and the viscosity was determined at 23C and 60 rpm with a Brookfield Model LV digital viscometer equipped with a number 1 or number 2 disc spindle. Viscosity readings were taken after the spindle had spun for exactly 10 s.

PDI analysis (Ba 10-65, AOCS 1990): Dispersed duplicate 20-g portions of each sample in 300 ml of distilled water at 25 & 1C. The dispersions were blended for 10 min at 8,500 rpm, poured into a 600-ml beaker and allowed to settle for 5 min. The upper layer of liquid was decanted into a 50-ml glass centrifuge tube and centrifuged for 10 min at 2900 rpm. The protein content was determined in 250 mg of supernatant (% water dispersible protein) and 250 mg of the original Euglena flour (% total protein) using a LECO combustion analyzer. The LECO instrument releases nitrogen from the sample by combustion in pure oxygen at high temperature. The freed nitrogen is measured by a thermal conductivity detector and converted to percent protein by the factor 6.25. The % PDI was calculated as follows:

$$\% PDI = \frac{\% water dispersible protein \times 100}{\% total protein}$$

Water Absorption: 10 mLs of water was added to 0.5 g of protein in a 13-ml Sarstedt graduated plastic test tube. The mixture was sonicated for 30 s at an output setting of 5 to disperse the sample. The mixture was held at 24° C. for 30 min, and then centrifuged at 2000 rpm for 25 min. The volume of free water was measured and the retained water was computed and reported as ml of water (+/− 0.1 ml) absorbed per g of flour.

Fat Absorption: A 3-ml portion of peanut oil was added to 0.5 g of protein in a 13-ml graduated plastic test tube. The contents were sonicated for 1 min at an output setting of 5 to disperse the sample. After holding at 24° C. for 30 min, the tube was centrifuged at 2000 rpm for 25 min. The volume of free oil was measured and the oil retained in the flour pellet was expressed as ml absorbed (+/− 0.1 ml) per g of flour.

Gel Strength: The “torsion test” is a common test used to evaluate gel strengths. An appropriately sized, and shaped gel is twisted in a rheometer until the gel either breaks, or it is ruptured. The amount of the force that caused the cross-section to rupture is then calculated and can be measured against other sensory results.

The strength of a gel is affected by temperature, pH, and the amount of the protein derivative in the food product. The gel strength of the food product comprising protein flour, protein concentrate and/or protein isolate can be measured by a tensiometer. The gel strength can also be measured by a texture analyzer, such as TA.XT Express or TA.XTPlus (Texture Technologies), FTC Texture Analyzer (Food Technology Corporation), and LFRA texture analyzer (Brookfield Engineering), which through compression and tensile data, can measure a number of physical properties, including tensile strength, i.e. a measurement of the force required to pull the gelatinous or “gelled” food product to the point where it breaks. Texture analyzers also test the crunchiness, gumminess, adhesiveness, chewiness, and general texture of many smaller things from animal crackers to zucchini. Texture analyzers measure tensile strength (i.e. in lb/int or psi) and compressive strength (i.e. psi or MPa) of materials. The principle of a texture measurement system is to physically deform a test sample in a controlled manner and measure its response. The characteristics of the force response are as a result of the sample's mechanical properties, which correlate to specific sensory texture attributes. A texture analyzer applies this principle by performing the procedure automatically and indicating the results visually on a digital numerical display, or screen.

Solubility: Many solubility tests are based on suspending and stirring a known amount of protein in a buffered solution, followed by centrifugation to remove insoluble components with subsequent protein analysis (coloimetric or Kjeldahl) of the supernatant.

Methods for Testing Water Holding Capacity: Centrifuge: rapidly rotating device applies centrifugal force to the components in order to force separation. As such, fluids of different densities become separated, as do liquids from solids.

Press Method: the water holding capacity of the food product is calculated based off of the weight of the substance after it has been pressed.

A Near Infrared Spectroscopy analyzes bulk, high-moisture samples in a non-destructive manner.

A SERS (Surface-enhanced Raman Spectroscopy) would be utilized to detect the presence of harmful or toxic compounds in foods.

A Titrator would be used to test for acidity and salt in the food products.

A Viscometer would be used for testing the viscosity of the developed food products, effectively delivering results related to mouth feel, how a product will react to temperature changes, as well as the spread ability of the product.

A Bostwick Consistometer will provide results related to the consistency of a food product.

The disclosures of each and every patent, patent application, publication, and accession number cited herein are hereby incorporated herein by reference in their entirety.

Preferences and options for a given aspect, feature, embodiment, or parameter of the invention should, unless the context indicates otherwise, be regarded as having been disclosed in combination with any and all preferences and options for all other aspects, features, embodiments, and parameters of the invention.

While present disclosure has been disclosed with reference to various embodiments, it is apparent that other embodiments and variations of these may be devised by others skilled in the art without departing from the true spirit and scope of the disclosure. The appended claims are intended to be construed to include all such embodiments and equivalent variations.

1. A composition comprising about 5% to about 100% Euglena biomass.

2. The composition of claim 1, wherein the Euglena biomass is about 10% to about 75% protein by dry weight.

3. The composition of claim 1, wherein the Euglena biomass was derived from Euglena cultured heterotrophically.

4. The composition of claim 1, wherein the Euglena biomass is predominantly intact Euglena cells.

5. The composition of claim 4, wherein the Euglena biomass is a microalgal flour.

6. The composition of claim 1, wherein a protein concentrate is isolated from the Euglena biomass.

7. The composition of claim 6, wherein a protein isolate is isolated from the Euglena biomass or from the protein concentrate.

8. The composition of claim 1, wherein an oil is extracted from the Euglena biomass.

9. The composition of claim 1, wherein the Euglena biomass is added to a food product wherein the food product comprises about 0.1% to about 99% Euglena biomass and an edible ingredient.

10. (canceled)

11. The composition of claim 9, wherein the Euglena biomass is selected from the group consisting of a microalgal flour, a protein concentrate, and a protein isolate.

12. The composition of claim 11, wherein the protein concentrate or the protein isolate is at least 40% protein.

13. The composition of claim 9, wherein the food product is selected from the group consisting of sauce, tea, candy, cookies, cereals, breads, fruit mixes, fruit salads, salads, snack bars, protein bars, fruit leather, yogurt, health bars, granola, smoothies, soups, juices, cakes, pies, shakes, ice cream, protein beverages, nutritional beverages, animal analogues, health drinks, cheese, milk, casein replacement, whip cream, non-dairy creamer, and combinations thereof.

14. (canceled)

15. The composition of claim 9, wherein the food product is an animal analogue selected from the group consisting of a meat analogue, sausage analogue, pepperoni/cured meat analogue, chicken analogue, turkey analogue, pork analogue, bacon analogue, beef analogue, tofu replacement, ground beef analogue, jerky analogue, an egg analogue, egg replacement, hard-boiled egg replacement, powdered egg replacement, liquid egg replacement, frozen egg replacement, salad dressing, mayonnaise, and combinations thereof.

16. (canceled)

17. The composition of claim 9, wherein the food product is an extruded product selected from the group consisting of a protein crisp, a cracker, a bar, pretzels, seaweed-like snack, cereal, pasta, crisps, puffs, oatmeal, cookies, and combinations thereof.

18. (canceled)

19. The composition of claim 9, wherein the food product is a protein bar.

20.-45. (canceled)

46. The composition of claim 1, wherein the Euglena is selected from Euglena gracilis, Euglena sanguinea, Euglena deses, Euglena mutabilis, Euglena acus, Euglena viridis, Euglena anabaena, Euglena geniculata, Euglena oxyuris, Euglena proxima, Euglena tripteris, Euglena chlamydophora, Euglena splendens, Euglena texta, Euglena intermedia, Euglena polymorpha, Euglena ehrenbergii, Euglena adhaerens, Euglena clara, Euglena elongata, Euglena elastica, Euglena oblonga, Euglena pisciformis, Euglena cantabrica, Euglena granulata, Euglena obtusa, Euglena limnophila, Euglena hemichromata, Euglena variabilis, Euglena caudata, Euglena minima, Euglena communis, Euglena magnifica, Euglena terricola, Euglena velata, Euglena repulsans, Euglena clavata, Euglena lata, Euglena tuberculata, Euglena contabrica, Euglena ascusformis, Euglena ostendensis, or combinations thereof.

47. The composition of claim 9, wherein the Euglena biomass is a dried Euglena biomass.

48. The composition of claim 9, wherein the Euglena biomass is a wet Euglena biomass.

49. The composition of claim 9 further comprising a flavoring, masking agent and/or additional ingredients.

50. (canceled)
